(19) United States
(12) Patent Application Publication
(10) Pub. No.: US 2006/0228404 A1
(43) Pub. Date: Oct. 12, 2006

(54) Compositions and methods for treatment of hypertrophic tissues

(76) Inventors: Daniel G. Anderson, Framingham, MA (US); Robert S. Langer, Newton, MA (US); Robert F. Padera JR., Milton, MA (US); Weidan Peng, Haverford, PA (US); Janet A. Sawicki, Newton Square, PA (US)

Correspondence Address: CHOATE, HALL & STEWART LLP, TWO INTERNATIONAL PLACE, BOSTON, MA, 02110, US

(21) Appl. No.: 11/256,452

(22) Filed: Oct. 21, 2005

Related U.S. Application Data
(63) Continuation-in-part of application No. 11/074,323, filed on Mar. 4, 2005.
(60) Provisional application No. 60/550,912, filed on Mar. 4, 2004.
(60) Provisional application No. 60/620,886, filed on Oct. 21, 2004.

(51) Int. Cl. A61K 48/00; A61K 31/785; A61K 9/127; C12N 15/88

(52) U.S. Cl. 424/450; 424/78.27; 514/44; 977/907; 435/458

(57) ABSTRACT

The present invention provides compositions and methods for treatment of conditions and diseases associated with excessive or inappropriate noncancerous tissue growth. In certain embodiments of the invention the compositions and methods are used for treatment of benign prostatic hyperplasia. In certain embodiments of the invention the composition comprises a tissue-selective delivery vehicle. In certain embodiments of the invention the compositions comprise an expression vector that encodes a cytotoxic polypeptide, wherein expression of the cytotoxic polypeptide is under control of a prostate-specific regulatory element. In certain embodiments of the invention the compositions comprise an expression vector in which expression of a recombinase is under control of a prostate-specific regulatory element, and a recombination event mediated by the recombinase is required for expression of the cytotoxic polypeptide.

### CROSS-REFERENCE TO RELATED APPLICATIONS

This application is a continuation-in-part of U.S. patent application Ser. No. 11/074,323 and PCT application PCT/US05/007001, both filed Mar. 4, 2005, which claim priority to U.S. Provisional Patent Application No. 60/550,912, filed Mar. 4, 2004. This application claims priority to and the benefit of U.S. provisional patent application 60/620,886, filed Oct. 21, 2004. All of these patent applications are incorporated herein by reference.

### GOVERNMENT SUPPORT

The United States Government has provided grant support utilized in the development of the present invention. In particular, National Institutes of Health grant number CA90841 has supported development of this invention. The United States Government may have certain rights in the invention.

### BACKGROUND OF THE INVENTION

A diverse array of diseases and clinical conditions are characterized by tissue hypertrophy. Among these, cancer is probably of most significance, and an immense amount of effort has been devoted to attempts to identify effective therapies for various cancer types. Since cancer is a disease featuring uncontrolled cell proliferation, many currently approved therapeutic agents are cytotoxic or cytostatic. In many cases these agents act by targeting dividing cells. While this approach confers a degree of specificity, it is typically the case that a number of other cell and tissue types in addition to the cancerous cells and tissues are adversely affected, frequently resulting in severe and often dose-limiting systemic and/or local side effects. In the context of a disease such as cancer, which is frequently lethal, such side effects are often considered acceptable.

A number of other significant diseases and clinical conditions are caused at least in part by, or feature, excessive or unwanted tissue growth. For example, benign prostatic hyperplasia (BPH), also referred to as benign prostatic hypertrophy, is one of the most common diseases of aging men, with a prevalence of greater than 50% by 60 years of age and as high as 90% by age 85 (1). BPH can be associated with disturbing lower urinary tract symptoms (LUTS) that can greatly diminish a patient's quality of life by interfering with daily activities and sleep.

Current treatments for BPH include both surgical and medical approaches. For most patients, the standard of care for treatment of BPH associated with moderate to severe lower urinary tract symptoms is transurethral resection of the prostate (TURP). Open prostatectomy is also an option. However, both operations are associated with significant morbidity. In addition, many patients with BPH are elderly and/or otherwise in poor health and may not be suitable surgical candidates. A variety of less invasive therapies for BPH also exist, including transurethral incision of the prostate, transurethral needle ablation of the prostate (TUNA), transurethral laser coagulation or vaporization, ultrasound, and injection with absolute ethanol or hyperosmolar sodium chloride. A number of these are considered experimental. Furthermore, postoperative bleeding or damage to neighboring healthy tissue resulting from high energy heat treatments or disseminating injectables can occur with these therapies. Medical therapies such as alpha-adrenergic blockers, 5-alpha reductase inhibitors, and combinations of these are often effective but may be associated with significant side effects such as sexual dysfunction, asthenia, hypotension, headache, and others (1). In addition, medical therapies require the patient to take medications on an ongoing basis, a source of continuing expense and inconvenience.

Approaches to treating other noncancerous conditions featuring inappropriate or excessive tissue growth vary widely, depending upon the particular condition in question. As in the case of BPH, options may range from surgical intervention to medical therapy. In general, given the fact that most noncancerous conditions featuring inappropriate or excessive tissue growth are nonlethal, the potential for side effects and morbidity associated with therapy may be a relatively greater consideration in selecting an appropriate treatment than in the case of cancer. Therefore, there is a need in the art for therapies that are cell and/or tissue-specific, i.e., therapies that selectively target hypertrophic tisses, noncancerous tumors, etc. In order to avoid the potential morbidity associated with surgery, there is a need for minimally invasive treatments for such diseases and conditions. In particular, there is a need in the art for improved treatments for BPH.

### SUMMARY OF THE INVENTION

The present invention addresses these needs, among others. In one aspect, the invention provides a method for treating a disease or condition characterized by inappropriate or excessive noncancerous tissue growth comprising the steps of: (i) providing a subject in need of treatment for a disease or condition characterized by inappropriate or excessive noncancerous growth of a tissue; and (ii) administering a tissue-selective therapeutic composition to the subject in an amount effective to cause a reduction in the size of the tissue, wherein the composition does not comprise a viral delivery vehicle. In certain embodiments of the invention the composition is locally delivered. The tissue-selective therapeutic composition may comprise a polymeric delivery vehicle (polymer), such as a poly (beta amino ester). In certain embodiments of the invention the delivery vehicle is tissue-selective. In certain embodiments of the invention, a composition comprising a tissue-selective delivery vehicle has substantially no effect on striated muscle.

In certain embodiments of the invention the composition comprises a polynucleotide, which may be expressed in a tissue-specific manner. In some embodiments the polynucleotide comprises a tissue-specific regulatory element specific for the tissue, operably linked to a nucleic acid that encodes a therapeutic polypeptide, e.g., a cytotoxic or cytostatic polypeptide. In other embodiments the polynucleotide comprises (i) a tissue-specific regulatory element specific for the tissue, operably linked to a nucleic acid that encodes a site-specific recombinase; (ii) a second regulatory element and a nucleic acid that encodes a therapeutic polypeptide, e.g., a cytotoxic or cytostatic polypeptide, wherein the second regulatory element is not operably linked to the nucleic acid; and (iii) sites that are recognized by the site-specific recombinase and are so positioned that activity of the recombinase results in a recombination event that places the second regulatory element and the nucleic acid into operable linkage so that the nucleic acid is transcribed.

The invention provides methods for treatment of benign prostatic hyperplasia (BPH), in which case the tissue-specific regulatory element is expressed in prostate gland tissue. For example, the invention provides a method for treating BPH comprising steps of: (a) providing an individual in need of treatment for BPH; and (b) administering to the individual a composition comprising a polynucleotide comprising a prostate specific regulatory element and a nucleic acid that encodes a therapeutic polypeptide, e.g., a cytotoxic or cytostatic polypeptide, wherein the composition either (i) does not comprise a viral delivery vehicle; or (ii) is locally delivered to noncancerous prostate gland tissue; or (iii) does not comprise a viral delivery vehicle and is locally delivered to noncancerous prostate gland tissue. Local delivery may be achieved, for example, by trans-urethral injection.

In another aspect, the invention provides a tissue-selective composition for the treatment of a disease or condition characterized by inappropriate or excessive noncancerous tissue growth, wherein the tissue-selective composition comprises a therapeutic agent, e.g., a cytotoxic or cytostatic agent or a polynucleotide that encodes a cytotoxic or cytostatic polypeptide, and does not comprise a viral delivery vehicle. In certain embodiments of the invention the composition comprises a polymeric delivery vehicle, e.g., a poly (beta amino ester). Either the therapeutic agent or the polymeric delivery vehicle, or both, are tissue-selective in various embodiments of the invention. Certain compositions comprising a tissue-selective delivery vehicle have substantially no effect on striated muscle but do affect one or more other tissues, e.g., epithelial cells, smooth muscle cells, etc.

The invention specifically provides tissue-selective compositions for treatment of BPH and kits comprising the compositions. The kits may further comprise a means for achieving local delivery to the prostate gland, e.g., a device for performing trans-urethral injection.

The methods and compositions of the invention may be used for treatment of a variety of conditions and diseases including, but not limited to, benign prostatic hyperplasia, gingival hyperplasia, obesity, hyperthyroidism, Graves' ophthalmopathy, a benign tumor, a bunion, a cyst, a fibroid, a scar, excessive breast size, and presence of undesirable tissue of a wide variety of different tissue types.

This application refers to various patents and publications. The contents of all of these are incorporated by reference. In addition, the following patent applications and publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Campbell, M. F., Walsh, P. C., and Retik, A. B., Campbell's Urology, 8^{th} ed., W. B. Saunders, Philadelphia, 2002; and Hardman, J., Limbird. E., Gilman, A. (Eds.), Goodman and Gilman's The Pharmacological Basis of Therapeutics, 10^{th} Ed. McGraw Hill, 2001; U.S. provisional patent applications 60/239,330, filed Oct. 10, 2000, 60/305,337, filed Jul. 13, 2001, and 60/550,912, filed Mar. 4, 2004, and U.S. Ser. Nos. 09/969,431, filed Oct. 2, 2001, and 10/446,444, filed May 28, 2003. In case of a conflict between the specification and a document incorporated by reference, the specification shall control. The determination of whether a conflict or inconsistency exists is within the discretion of the inventors and can be made at any time.

### BRIEF DESCRIPTION OF THE DRAWING

FIG. 1A shows general formulas for a poly (beta amino ester). FIG. 1B shows amino and acrylate monomers used to create a poly(β-amino ester) library. FIG. 1C shows synthesis and structure of polymer C32. FIG. 1D shows a variety of poly (beta amino esters) that may be used in the present invention.

FIG. 2 shows measurements of the in vitro transfection efficiency of various poly(β-amino esters). The transfection efficiency of polymers synthesized at the optimal amine:acrylate ratio and at the optimal polymer:DNA ratio is shown. Polymers were synthesized at 6 amine:acrylate ratios (1, 1.025, 1.05, 1.1, 1.2, and 1.3), unless marked with an arrow, in which case they were synthesized at 12 amine:acrylate ratios (0.6, 0.8, 0.9, 0.95, 0.975, 1, 1.025, 1.05, 1.1, 1.2, 1.3, and 1.4). Polymers were synthesized at 95° C. in the absence of solvent (blue bars) or at 60° C. in the presence of 2 ml DMSO (red bars). The amine:acrylate ratio of the optimal polymer is listed next to the monomer composition.

FIG. 3 shows cytotoxicity measurements of various poly(β-amino esters) and comparison with cytotoxicity of PEI. COS-7 cells were incubated with polymer in Opti-MEM® medium for one hour and metabolic activity was measured 24 hours later. Measurements were performed in quadruplicate, and standard deviations are shown for C32 and PEI.

FIGS. 4A and 4B show images and quantification of tumor transfection by polymer:DNA complexes in vivo. Xenografts of PC3 human prostate tumor cells were injected with 1) C32 (1.2/1 amine:acrylate ratio) complexed to pCAG/luc DNA at a 30:1 polymer:DNA ratio, 2) In vivo Jet PEI® complexed to pCAG/luc DNA according to manufacturer's instructions, 3) naked pCAG/luc DNA or 4) buffer. Two days following transfection, mice were imaged and bioluminescence was quantified. A. Pseudocolor images representing light emitted from tumors superimposed over grayscale reference image of representative mice from each group of five. B. Quantification of the emitted photons from each tumor. Horizontal bars indicate the mean value for each treatment group.

FIGS. 5A and 5B show images and quantification of muscle transfection by polymer:DNA complexes in vivo. Healthy muscle was injected with 1) C32 (1.2/1 amine:acrylate ratio) complexed to pCAG/luc DNA at a 30:1 polymer:DNA ratio, 2) In vivo Jet PEI® complexed to pCAG/luc DNA according to the manufacturer's instructions, and 3) naked pCAG/luc DNA. Two, six, and twenty days following transfection, mice were imaged and bioluminescence was quantified. A. Pseudocolor images representing light emitted from muscle superimposed over grayscale reference image of representative mice from each group of five. B. Quantification of the emitted photons from each injected muscle. Horizontal bars indicate the mean value for each treatment group.

FIG. 6 shows histological analysis of muscle and tumors following transfection with polymer:DNA (pGAG/luc) complexes. Photomicrographs of hematoxylin and eosin stained sections of muscle (A,B) and tumor (C,D) taken using 10× objective. A. Muscle injected with C32/DNA shows no pathology. B. Muscle injected with PEI/DNA shows damaged myocytes with calcifications, indicated with arrows. C. Uninjected tumor control. D. Tumor injected with C32/DNA shows no histological differences from control tumor.

FIG. 7 shows inhibition of luciferase activity by C32-delivered DNA encoding DT-A. LNCaP cells were incubated with C32/DNA complexes for 1 hour after which the medium was changed. Forty-eight hours later, cells were harvested, and protein extracts were prepared and assayed for luciferase activity. The DNA constructs used are indicated below each bar. luc=C32-pCAG/luc, EGFP=C32-pRSV/FRT2PSA.FLP/EGFP, DT-A=C32-pRSV/FRT2PSA.FLP/DT-A. The experiment was repeated three times.

FIG. 8 shows tumor growth following intratumoral injection of C32-pRSV/FRT2PSA.FLP/DT-A or C32-salmon sperm DNA nanoparticles. Nanoparticles were injected on day 0 and then every other day for a total of 6 injections (50 mg DNA/injection, 30:1 polymer:DNA ratio). Tumor volume was measured using calipers on day 0 and day 14. Fold increase in tumor volume is the ratio of these two measurements. Horizontal bars indicate the mean value for each treatment group.

FIGS. 9A-9D show images of transfection of a variety of healthy tissues by polymer:DNA complexes in vivo. Healthy mouse tissue was injected with C32 (1.2/1 amine:acrylate ratio) complexed to 50 μg pCMV/luc DNA at a 30:1 polymer:DNA ratio. Forty-eight hours following transfection, mice were imaged and bioluminescence was quantified. Mice were then sacrificed and imaged again after opening the abdominal cavity. Various organs and tissues were removed and imaged individually. A. Images obtained from various tissues following injection of C32:pCAG/luc complexes into ventral lobe of prostate. Upper left panel: pseudocolor image representing light emitted from various tissues prior to sacrifice superimposed over grayscale image of mouse; Middle left panel: grayscale image of mouse following sacrifice; Lower left panel: pseudocolor images representing light emitted from various tissues following sacrifice superimposed over grayscale images of tissues; Right panel: pseudocolor images representing light emitted from various tissues following dissection superimposed over grayscale images of tissues B. Images obtained from various tissues following injection of C32:pCMV/luc complexes into spleen. Upper left panel: pseudocolor image representing light emitted from various tissues prior to sacrifice superimposed over grayscale image of mouse; Middle left panel: grayscalse image of mouse following sacrifice; Lower left panel: pseudocolor image representing light emitted from various tissues following sacrifice; Right panel: pseudocolor images representing light emitted from various tissues following dissection superimposed over grayscale images of tissues. C. Images obtained from various tissues following injection of C32:pCMV/luc complexes into left lobe of liver. Upper left panel: pseudocolor image representing light emitted from various tissues prior to sacrifice superimposed over grayscale image of mouse; Middle left panel: grayscale image of mouse following sacrifice; Lower left panel: pseudocolor image representing light emitted from various tissues following sacrifice superimposed over grayscale image of mouse; Right panel: pseudocolor images representing light emitted from various tissues following dissection superimposed over grayscale images of tissues. D. Images obtained from various tissues following injection of C32:pCMV/luc complexes into left testis. Upper left panel: pseudocolor image representing light emitted from various tissues prior to sacrifice superimposed over grayscale image of mouse; Middle left panel: grayscale image of mouse following sacrifice; Lower left panel: pseudocolor image representing light emitted from various tissues following sacrifice; Right panel: pseudocolor images representing light emitted from various tissues following dissection superimposed over grayscale images of tissues.

FIG. 10 shows a photograph of a mouse prostate gland 5 days after injection of a PSA/DT-A:C32 complex into the right ventral lobe. Labels indicate the left ventral (LV), right ventral (RV), left lateral (LL), and right lateral (RL) lobes. The right ventral lobe is significantly reduced in size compared to the untreated left ventral (LV) lobe. The left lateral (LL) and right lateral (RL) lobes are of equal size.

FIGS. 11A-11C show schematic diagrams of a variety of nucleic acid constructs. FIG. 11A shows schematic diagrams of pRSV/FRT2PSA.FLP/DT-A (top) and pRSV/FRT2PSA.FLP/EGFP (middle). Transcription of the FLP coding sequence is driven by the PSE-BC promoter/enhancer and proceeds from right to left. Recombination catalyzed by FLP places the Rous sarcoma virus (RSV) promoter in operable association with the sequence coding for DT-A or EGFP, respectively, as shown for EGFP (bottom), where PSA represents the prostate-specific PSE-BC promoter/enhancer element. The transgene is in a vector with Ad sequences to allow creation of adenovirus but could be housed in any of a wide range of plasmid vectors and/or used for the creation of other viral vectors, e.g., lentivirus, retrovirus, adeno-associated virus, etc. FIG. 11B shows a schematic diagram of pCAG/Luc, a reporter construct in which the CAG promoter/enhancer drives transcription of a sequence encoding luciferase. FIG. 11C shows schematic diagrams of pRSV/FRT2neo/DT-A (top), a construct in which a ubiquitous promoter (RSV) is separated from a coding sequence for DT-A by a sequence encoding a selectable marker flanked by two FRT sites, and pBSE-BC/FLP (bottom), a construct in which transcription of a nucleic acid that encodes FLP recombinase is driven by a prostate-specific PSE-BC promoter/enhancer.

FIGS. 12A and 12B show photographs of the InjecTx™ device, which can be used to inject a composition into the prostate gland. FIG. 12A shows the device. FIG. 12B shows enlarged views of the handle and injection needle. From 59.

FIG. 13 shows use of the InjecTx device to inject a composition into hypertrophic prostate gland tissue. From 59.

FIGS. 14A and 14B show expression of nanoparticle-delivered and naked pCAG/luc DNA in prostate and other organs following intraprostatic injection. A. 50 μg DNA, either complexed with C32 to form nanoparticles(left) or naked (right), was injected into the right ventral lobe of the prostate. Two days after injection, mice were imaged in toto, euthanized and then organs and tissues were removed and imaged ex vivo. Pseudocolor images representing emitted light are superimposed over grayscale reference images of whole mice and different organs and tissues. RV-LP, right ventral/lateral prostate (* indicates this was the injected lobe); DP, dorsal prostate; AP, anterior prostate; S, ventral skin near injection site; B, bladder; RT, right testis; RF, fat on right near prostate; RSV, right seminal vesicle; H, heart; Lu, lung; L, liver; Sp, spleen; K, kidney. Relative light units/pixel are indicated in the color scale bar. For each treatment group, the analysis was performed on 5 mice. Images are representative. B. Quantification of the emitted photons from prostates injected with nanoparticle-delivered DNA or with naked DNA. The number of photons emitted from the nanoparticle-injected prostates is significantly higher than the number emitted from prostates injected with naked DNA.

FIGS. 15A-15C show morphological, TUNEL, and histological evidence for cell death following intraprostatic injection of PSA/DT-A nanoparticles in mice. In each panel, the top photograph is a representative picture of the mouse prostate in situ 7 days following injection of (A) DT-A nanoparticles, (B) PSA/Fluc nanoparticles, and (C) PBS. LV, left ventral lobe; RV, right ventral lobe; * indicates lobe that was injected; B, bladder. The bottom composite figures in each panel show representative TUNEL analysis (and DAPI staining of same section), and H & E stained section at 40× magnification. Apoptotic cells appear green.

FIG. 16 shows expression of CFP and EGFP in prostate sections of double transgenic (K5/CFP+PSA/EGFP) mice. Basal cells in the prostate epithelium fluoresce blue; luminal cells fluoresce green. Sections are from non-injected prostate (40×), and from prostates injected with C32-PSA/DT-A nanoparticles (10×), C32-PSA/Fluc nanoparticles (10×), and PBS (10×).

FIGS. 17A and 17B show luciferase specific activity following C32-nanoparticle delivery of DNA to human primary prostate cell lines. Epithelial, stromal, and smooth muscle cells were transfected with nanoparticles, and luciferase activity was assayed 48 hours later. A.C32-CAG/Fluc (black bars); C32-PSA/Fluc (cross-hatched bars). B. Cells transfected with C32-CAG/Fluc, then 3 hr later with C32-PSA/Flp (dark shaded bars), or with C32-PSA/DT-A (light shaded bars).

### DETAILED DESCRIPTION OF CERTAIN EMBODIMENTS OF THE INVENTION

I. Definitions

The following definitions are of use in understanding the invention. Other definitions are included in the specification.

Antibody: In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. Antibodies, antibody fragments, and/or protein domains comprising an antigen binding site may be generated and/or selected in vitro, e.g., using techniques such as phage display (Winter, G. et al.1994. Annu. Rev. Immunol. 12:433-455, 1994), ribosome display (Hanes, J., and Pluckthun, A. Proc. Natl. Acad. Sci. USA. 94:4937-4942, 1997), etc. In various embodiments of the invention the antibody is a “humanized” antibody in which for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

Approximately: As used herein, the terms approximately or about in reference to a number are generally taken to include numbers that fall within a range of 5% in either direction (greater than or less than) of the number unless otherwise stated or otherwise evident from the context (except where such number would exceed 100% of a possible value).

Biocompatible: A material is considered biocompatible with respect to cells if it is substantially non-toxic to cells in vitro, e.g., if its addition to cells in culture results in less than or equal to 20% cell death. A material is considered biocompatible with respect to a recipient, if it is substantially non-toxic to the recipient's cells in the quantities and at the location used, and also does not elicit or cause a significant deleterious or untoward effect on the recipient's body, e.g., an immunological or inflammatory reaction, unacceptable scar tissue formation, etc.

Biodegradable: Capable of being broken down physically and/or chemically within cells or within the body of a subject, e.g., by hydrolysis under physiological conditions, by natural biological processes such as the action of enzymes present within cells or within the body, etc., to form smaller chemical species which can be metabolized and, optionally, reused, and/or excreted or otherwise disposed of. Preferably a biodegradable compound is biocompatible.

Cell type: A cell type is a category of cells that share at least some morphological and/or functional characteristics. A cell type may be defined with different levels of specificity and cells of a particular type may be found in only a single organ or in multiple different organs. For example, cell types that are found in many different organs include epithelial cells, stromal cells, fibroblasts, and macrophages. Epithelial cells found in glands such as the prostate gland include luminal epithelial cells and basal cells. Other cell types include smooth muscle cells and striated muscle cells. Some cell types are found in a specific organ or tissue type. Examples include hepatocytes, chondrocytes, osteoblasts, osteoclasts, adipocytes, etc. A cell type that is found in more than one different organ or tissue may be further classified into more narrowly defined cell types based on the organ or tissue in which they are found and/or based on one or more functional or morphological characteristics shared by some but not all cells of that type.

Cell type specific marker: A cell type specific marker is a molecular entity or portion thereof that is present at a higher level on or in a particular cell type or cell types of interest than on or in many other cell types. The molecular entity can be, e.g., a polypeptide, mRNA, lipid, or carbohydrate. In some instances a cell type specific marker is present at detectable levels only on or in a particular cell type of interest. However, it will be appreciated that useful cell type specific markers need not be absolutely specific for the cell type of interest. For example, certain CD molecules are present on the cells of multiple different types of leukocytes. In general, a cell type specific marker for a particular cell type is expressed at levels at least 3 fold greater in that cell type than in a reference population of cells which may consist, for example, of a mixture containing cells from a plurality (e.g., 5-10 or more) of different tissues or organs in approximately equal amounts. More preferably the cell type specific marker is present at levels at least 4-5 fold, between 5-10 fold, or more than 10-fold greater than its average expression in a reference population. Preferably detection or measurement of a cell type specific marker makes it possible to distinguish the cell type or types of interest from cells of many, most, or all other types. In general, the presence and/or abundance of most markers may be determined using standard techniques such as Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein microarray analysis, mass spectrometry, etc.

Typically a determination of whether a molecular entity is a useful cell type specific marker for one or more particular cell types will be made by comparing expression of the marker in different cell types found in the same species. However, it will be appreciated that homologs of many molecular entities exist in multiple different species, and once a cell type specific marker is found in a particular species, a homologous marker will typically exist in related species and will frequently have the same or a similar cell type specificity in such species as in the species in which it was identified. When using a cell type specific marker to distinguish between cells of different types in a particular species, it will often be desirable to utilize the particular homolog of the cell type specific marker that is found in cells of that species. For example, it may be desirable to utilize a murine polypeptide as a cell type specific marker to distinguish between different types of mouse cells and a human homolog of the same polypeptide as a cell type specific marker to distinguish between different types of human cells.

Cell type specific regulatory element: A cell type specific regulatory element is a regulatory element that is active at a significantly higher level in a particular cell type or cell types of interest than in many other cell types, e.g., a regulatory element that directs transcription of an operably linked nucleic acid at a significantly higher level in a particular cell type or cell types of interest. In some instances a cell type specific regulatory element is active (e.g., drives transcription) at detectable levels only in a particular cell type of interest. However, it will be appreciated that cell type specific regulatory elements need not be absolutely specific for the cell type of interest in the sense of having detectable activity on in that cell type. For example, a number of promoters that are considered cell type specific in the art are “leaky” and direct expression in many cell types, albeit at a lower level than in the cell type for which they are considered specific.

In general, a regulatory element may either increase or decrease expression of an operably linked sequence. Preferably a cell type specific regulatory element that is specific for a particular cell type affects (e.g., increases or decreases) expression to an extent at least 3 fold greater in that cell type than in a reference population of cells, or in a number of different individual cell types (e.g., at least 3 other cell types, preferably at least 4 other cell types, more preferably between 5 and 10 other cell types, etc. For example, a cell type specific regulatory element may direct expression in that cell type at a level at least 3 fold greater than the level at which it directs expression in a reference population of cells (or in a panel of different individual cell types) or may increase a basal level of expression (e.g., a level in the absence of any enhancing elements) by at least 3 fold in that cell type relative to the level at which it increases basal expression in a reference population of cells (or in a panel of different individual cell types). The reference population may consist, for example, of a mixture containing cells from a plurality (e.g., 5-10 or more) of different tissues or organs in approximately equal amounts. More preferably the cell type specific regulatory element affects expression at levels at least 4-5 fold, between 5-10 fold, or more than 10-fold greater in a cell type of interest than in a reference population. In general, the level of expression may be determined using standard techniques for measuring mRNA or protein.

Typically a determination of whether a regulatory element is a useful cell type specific regulatory element will be made by comparing expression directed by that regulatory element in different cell types found in the same species. However, it will be appreciated that homologs of many regulatory elements exist in multiple different species, and once a cell type specific regulatory element is found in a particular species, a homologous regulatory element will typically exist in related species and will frequently have the same or a similar cell type specificity in such species as in the species in which it was identified. When using a cell type specific regulatory element to direct expression in cells of one or more types in a particular species, it will often be desirable to utilize the particular homolog of the cell type regulatory element that is found in cells of that species. For example, it may be desirable to utilize a regulatory element from a murine gene as a cell type specific regulatory element in mouse cells and a regulatory element from human homolog of the same gene as a cell type specific regulatory element in human cells.

Effective amount: In general, the “effective amount” of an active agent refers to the amount necessary to elicit a desired biological response. As will be appreciated by those of ordinary skill in this art, the effective amount of an agent may vary depending on such factors as the desired biological endpoint, the agent to be delivered, the composition of an encapsulating matrix, the target tissue, etc. For example, the effective amount of a composition for treatment of inappropriate or excessive noncancerous tissue growth preferably reduces such growth in a clinically significant manner. The reduction may be expressed, for example, in either absolute terms or relative to the initial size of the tissue. An effective amount may measurably alleviate one or more symptoms of inappropriate or excessive tissue growth. An effective amount may measurably reduce the severity of one or more clinical or laboratory signs of inappropriate or excessive tissue growth. The alleviation or reduction can be measured using any suitable method including, but not limited to, (i) the use of standardized questionnaires assessing symptoms or overall quality of life or patient satisfaction with an outcome, (ii) physical examinations such as digital rectal examination of prostate gland size, (iii) imaging studies such as X-ray, ultrasound, CT scan, MRI, etc., (iv) measurement of the level of a serum or tissue marker whose level correlates with tissue hypertrophy, such as prostate specific antigen (PSA) in the case of BPH, etc.

Expression control sequence. An “expression control sequence” refers to a nucleotide sequence in a polynucleotide that regulates the expression (transcription and/or translation) of a nucleotide sequence operably linked thereto.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as tRNAs. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification). A gene is said to “encode” an RNA or polypeptide expression product.

Gene therapy vector. A “gene therapy vector” is a vector, as defined below, that comprises a template for transcription of a therapeutic nucleic acid molecule (e.g., an siRNA strand, shRNA strand, antisense RNA strand, ribozyme, or aptamer), or comprises a template for transcription of a nucleic acid molecule that is translated to produce a therapeutic polypeptide.

Hyprplasia: Hyperplasia refers to an increase in the volume of a tissue or organ caused by an increase in the number of cells, typically due to cell proliferation or reduction in cell death, or both.

Hypertrophy: Hypertrophy refers to an increase in the volume and/or mass of a tissue or organ. In most cases, the increase is caused at least in part by an increase in cell number (hyperplasia), an increase in cell size, or both. Hypertrophy may also be caused by or may involve deposition or collection of noncellular material such as lipid, extracellular matrix components such as collagen and proteoglycans, etc.

Liposomes: Liposomes are artificial microscopic spherical particles formed by a lipid bilayer (or multilayers) enclosing an aqueous compartment. Liposomes are commonly used in molecular biology and medicine as a delivery vehicle for various types of molecules (such as proteins, small molecules, DNA, and RNA), including a number of different drugs and can be used for delivering the compositions of the invention.

Local delivery: Local delivery, in reference to delivery of a composition or device of the invention containing a therapeutic agent, refers to delivery that does not rely primarily upon transport of the agent to its intended target (cells, tissue, or organ) via the vascular system. The agent is delivered directly to its intended target or in the vicinity thereof, e.g. by injection or implantation of the composition or device containing the agent. Following local administration in the vicinity of a target site, the agent may diffuse to the intended target. If a composition or device is injected or implanted in the vicinity of a target tissue rather than directly into the target tissue, the distance between the site of injection or implantation will be selected so as to allow diffusion of the therapeutic agent to the target in effective amounts. Typically “in the vicinity” or “near” refers to locations within several centimeters or less (e.g., within 3-4 cm), typically 1 cm or less of at least a portion of a target tissue or organ. It will be understood that once having been locally delivered a fraction of a therapeutic agent (typically only a minor fraction of the administered dose) may enter the vascular system and be transported to another location, including to its intended target.

Operably linked: As used herein, “operably linked” or “operably associated” refers to a relationship between two nucleic acid sequences wherein the expression of one of the nucleic acid sequences is controlled by, regulated by, modulated by, etc., the other nucleic acid sequences, or a relationship between two polypeptides wherein the expression of one of the polypeptides is controlled by, regulated by, modulated by, etc., the other polypeptide. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport, stability, or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence, or a polypeptide that is operably linked to a second polypeptide, is covalently linked, either directly or indirectly, to such a sequence, although any effective three-dimensional association is acceptable.

Polynucleotide: “Polynucleotide” or “oligonucleotide” refers to a polymer of nucleotides. As used herein, an oligonucleotide is typically less than 100 nucleotides in length. A polynucleotide or oligonucleotide may also be referred to as a nucleic acid. Typically, a polynucleotide comprises at least three nucleotides. A nucleotide comprises a nitrogenous base, a sugar molecule, and a phosphate group. A nucleoside comprises a nitrogenous base linked to a sugar molecule. In a polynucleotide or oligonucleotide, phosphate groups covalently link adjacent nucleosides to form a polymer. The polymer may comprise or natural nucleosides found in DNA or RNA (e.g., adenosine, thymidine, guanosine, cytidine, uridine, deoxyadenosine, deoxythymidine, deoxyguanosine, and deoxycytidine), other nucleosides or nucleoside analogs, nucleosides containing chemically modified bases and/or biologically modified bases (e.g., methylated bases), intercalated bases, modified sugars, etc. The phosphate groups in a polynucleotide or oligonucleotide are typically considered to form the internucleoside backbone of the polymer. In naturally occurring nucleic acids (DNA or RNA), the backbone linkage is via a 3′ to 5′ phosphodiester bond. However, polynucleotides and oligonucletides containing modified backbones or non-naturally occurring internucleoside linkages can also be used in the present invention. Such modified backbones include ones that have a phosphorus atom in the backbone and others that do not have a phosphorus atom in the backbone. Examples of modified linkages include, but are not limited to, phosphorothioate and 5′-N-phosphoramidite linkages. See Kornberg and Baker, DNA Replication, 2nd Ed. (Freeman, San Francisco, 1992), Scheit, Nucleotide Analogs (John Wiley, New York, 1980), U.S. Patent Pub. No. 20040092470 and references therein for further discussion of various nucleotides, nucleosides, and backbone structures that can be used in the polynucleotides or oligonucleotides described herein, and methods for producing them. Typically a polynucleotide of this invention is DNA or RNA.

Polynucleotides and oligonucleotides need not be uniformly modified along the entire length of the molecule. For example, different nucleotide modifications, different backbone structures, etc., may exist at various positions in the polynucleotide or oligonucleotide. Polynucleotides may be single- or double-stranded. If single-stranded a polynucleotide may be the coding (sense) strand or non-coding (anti-sense) strand.

A polynucleotide may be provided by a variety of means known in the art. In certain embodiments, the polynucleotide has been engineered using recombinant techniques (for a more detailed description of these techniques, please see Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). A polynucleotide may also be obtained from natural sources and purified from contaminating components found normally in nature. The polynucleotide may be synthesized using enzymatic techniques, either within cells or in vitro. A polynucleotide may be chemically synthesized, e.g., using standard solid phase chemistry. A polynucleotide may be modified by chemical and/or biological means. In certain embodiments, these modifications lead to increased stability of the polynucleotide. Modifications include methylation, phosphorylation, end-capping, etc.

The term “polynucleotide sequence” or “nucleic acid sequence” as used herein can refer to the nucleic acid material itself and is not restricted to the sequence information (i.e. the succession of letters chosen among the five base letters A, G, C, T, or U) that biochemically characterizes a specific nucleic acid, e.g., a DNA or RNA molecule. A nucleic acid sequence is presented in the 5′ to 3′ direction unless otherwise indicated.

Polypeptide. “Polypeptide”, as used herein, refers to a polymer of amino acids. A protein is a molecule composed of one or more polypeptides. A peptide is a relatively short polypeptide, typically between about 2 and 60 amino acids in length. The terms “protein”, “polypeptide”, and “peptide” may be used interchangeably. Polypeptides used herein typically contain amino acids such as those that are naturally found in proteins. However, amino acids that are not naturally found in proteins (i.e., amino acids that either do or do do not occur in nature and that can be incorporated into a polypeptide chain), and/or amino acid analogs can also or alternatively be used. One or more of the amino acids in a polypeptide may be modified, for example, by the addition of a chemical entity such as a carbohydrate group, a phosphate group, a famesyl group, an isofamesyl group, a fatty acid group, a linker for conjugation, functionalization, or other modification, etc. Modifications may include cyclization of the peptide, the incorporation of D-amino acids, etc. Preferably the modification does not substantially interfere with the desired biological activity of the polypeptide. Polypeptides of use in this invention may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or using methods involving chemical ligation of synthesized peptides (see, e.g., Kent, S., J Pept Sci., 9(9):574-93, 2003 and U.S. Pub. No. 20040115774), or any combination of these. In certain embodiments of the present invention a polypeptide is synthesized in vivo in cells of an organism in which the polypeptide exerts a desired therapeutic effect.

The term “polypeptide sequence” or “amino acid sequence” as used herein can refer to the polypeptide material itself and is not restricted to the sequence information (i.e. the succession of letters or three letter codes chosen among the letters and codes used as abbreviations for amino acid names) that biochemically characterizes a polypeptide. A polypeptide sequence presented herein is presented in an N-terminal to C-terminal direction unless otherwise indicated.

Regulatory element or regulatory sequence: The term “regulatory element” or “regulatory sequence” in reference to a nucleic acid is generally used herein to describe a portion of nucleic acid that regulates one or more steps in the expression (particularly transcription, but in some cases other events such as splicing or other processing) of nucleic acid sequence(s) with which it is operably linked. The term includes promoters and can also refer to enhancers and other transcriptional control elements. Promoters are regions of nucleic acid that include a site to which RNA polymerase binds before initiating transcription and that are typically necessary for even basal levels of transcription to occur. Such elements often comprise a TATA box. Enhancers are regions of nucleic acid that encompass binding sites for protein(s) that elevate transcriptional activity of a nearby or distantly located promoter, typically above some basal level of expression that would exist in the absence of the enhancer. In some embodiments of the invention, regulatory sequences may direct constitutive expression of a nucleotide sequence (e.g., expression in most or all cell types under typical physiological conditions in culture or in an organism); in other embodiments, regulatory sequences may direct cell or tissue-specific and/or inducible expression. For example, expression may be induced by the presence or addition of an inducing agent such as a hormone or other small molecule, by an increase in temperature, etc. Regulatory elements may also inhibit or decrease expression of an operably linked nucleic acid. Such regulatory elements may be referred to as “negative regulatory elements”.

In general, the level of expression may be determined using standard techniques for measuring mRNA or protein. Such methods include Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein array analysis, mass spectrometry, etc. A convenient way to determine expression level is to place a nucleic acid that encodes a readily detectable marker (e.g., a fluorescent or luminescent protein such as green fluorescent protein or luciferase, an enzyme such as alkaline phosphatase, etc.) in operable association with the regulatory element in an expression vector, introduce the vector into a cell type of interest or into an organism, maintain the cell or organism for a period of time, and then measure expression of the readily detectable marker, taking advantage of whatever property renders it readily detectable (e.g., fluorescence, luminescence, alteration of optical property of a substrate, etc.). Comparing expression in the absence and presence of the regulatory element indicates the degree to which the regulatory element affects expression of an operably linked sequence.

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

Subject. “Subject”, as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

Target cell, tissue, or organ: A target cell, tissue, or organ is a cell, tissue, or organ to which a composition of the invention is to be delivered and/or in which the composition or an agent contained in the composition is to be active. Typically a target tissue or organ is one whose size (i.e., the value of one or more dimension) and/or volume is to be reduced or whose continued increase in size and/or volume is to be inhibited or prevented. If the reduction or inhibition of continued increase in size and/or volume takes place by reducing the number and/or proliferation of one or more cell types in a target tissue or organ, the cell type(s) is considered to be a target cell.

Therapeutic agent: “Therapeutic agent” refers to an agent (e.g., a polynucleotide, polypeptide, or small molecule) that is administered to a subject to treat a disease, disorder, or other clinically recognized condition that is harmful or undesirable to the subject, or for prophylactic purposes. The term “therapeutic agent” includes polynucleotides that encode therapeutic polypeptides, e.g., cytotoxic or cytostatic polypeptides such as those described herein.

Tissue growth: “Tissue growth” refers to an expansion or increase in at least one dimension of the tissue, typically resulting in an expansion or increase in the total volume of the tissue, relative to a previous state (e.g., a normal state) or relative to a desired state. The growth is typically due at least in part to proliferation of one or more cell types in the tissue (hyperplasia) or may be due at least in part to other causes of hypertrophy. In certain instances the dimensions of the tissue may fall within the normal range for the general population or may be considered normal given the subject's other physical characteristics (e.g., height, weight, sex), but may (i) cause symptoms and/or (ii) be displeasing to the subject. The tissue growth may simply be an increase in size associated with normal growth, e.g., growth to adulthood and may not be due to any specific disease process.

Treating: “Treating”, as used herein, refers to providing treatment, i.e, providing any type of medical and/or surgical management of a subject. The treatment can be provided in order to reverse, alleviate, inhibit the progression of, prevent or reduce the likelihood of a disease or condition, or in order to reverse, alleviate, inhibit or prevent the progression of, prevent or reduce the likelihood of one or more symptoms or manifestations of a disease or condition. “Prevent” refers to causing a disease or condition, or symptom or manifestation of such not to occur. Treating can include administering a composition or device of this invention to the subject following the development of one or more symptoms or manifestations indicative of a disease or condition such as BPH, e.g., in order to reverse, alleviate, reduce the severity of, and/or inhibit or prevent the progression of the condition and/or to reverse, alleviate, reduce the severity of, and/or inhibit or prevent the progression of one or more symptoms or manifestations of the disease or condition. A composition or device of this invention can be administered to a subject who has developed a disease or condition such as BPH or is at increased risk of developing such a disorder relative to a member of the general population that would normally be considered susceptible to developing the disorder (e.g., males in the case of BPH). A composition or device of this invention can be administered prophylactically, i.e., before development of any symptom or manifestation of the disease or condition. Typically in this case the subject will be at increased risk of developing the disease or condition relative to a member of the general population that would normally be considered susceptible to developing the disorder.

Tumor: An abnormal mass or lump of tissue, typically caused by excessive cell division. Tumors can be benign (non-cancerous) or malignant (cancerous). Benign and malignant tumors are typically distinguished on the basis of their clinical features and/or based on histopathology, cytogenetic features, immunological features, gene expression profile, etc. A benign tumor remains confined to a local area, typically within a fibrous capsule that separates it from surrounding normal tissue. Benign tumors generally do not infiltrate or invade adjacent tissues or spread to distant locations within the body (metastasize), and generally are not fatal. Benign tumors include fibromas, myxomas, lipomas, chondromas, osteomas, hemangiomas, lymphangiomas, non-invasive meningiomas, glomus tumors, leiomyomas, rhabdomyomas, papillomas, adenomas, nevi, hydatiform mole, mature teratomas, and dermoid cysts. A malignant tumor (cancer), typically spreads locally and/or to remote sites within the body, and is frequently fatal if untreated. Malignant tumors (cancers) are often poorly differentiated and frequently display variation in cell size and shape. Nuclei in malignant tumors often display atypical mitotic figures, abnormally large nuclei, and variations in nuclear size and shape. See, e.g., Cotran, R. S., Kumar, V., Collins, T., and Robbins, S. L., 7^{th} ed., Robbins Pathologic Basis of Disease, W. B. Saunders, 2004; and Devita, V. T., et al, (eds.) Cancer: Principles & Practice of Oncology, 6^{th} ed. Lippincott Williams & Wilkins (Feb. 15, 2001) and forthcoming December 2004 edition of this work for further information.

Vector: The term “vector” is used herein to refer to a nucleic acid or a virus or portion thereof (e.g., a viral capsid) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication, or may include sequences sufficient to allow integration of part of all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof (e.g., viral capsids) that can be used to introduce nucleic acid molecules into cells are referred to as viral vectors. Useful viral vectors include adenoviruses, retroviruses, lentiviruses, vaccinia virus and other poxviruses, herpex simplex virus, and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Where sufficient information is lacking it may, but need not be, supplied by a host cell or by another vector introduced into the cell.

Expression vectors are vectors that include regulatory sequence(s), e.g., a promoter, sufficient to direct transcription of an operably linked nucleic acid. Such vectors typically include one or more appropriately positioned sites for restriction enzymes, to facilitate introduction of the nucleic acid to be expressed into the vector.

II. Overview

The invention provides compositions and methods for treating a disease or condition characterized by inappropriate or excessive noncancerous tissue growth. The compositions comprise a tissue-selective or tissue-specific therapeutic agent, a tissue-selective or tissue-specific delivery vehicle, or both. The methods comprise administering a tissue-selective or tissue-specific therapeutic composition to the subject in an amount effective to cause a reduction in the size of the tissue and/or to inhibit or prevent continued increase in size of the tissue. By “reduction in size” is meant a decrease in the value of one or more dimensions of the tissue, typically resulting in a decrease in total volume of the tissue. If the target tissue is present in an organ, the volume of the organ will be reduced and/or continued increase in volume of the organ will be inhibited or prevented. By “increase in size” is meant an increase in the value of one or more dimensions of the tissue, typically resulting in an increase in total volume of the tissue. By “tissue-selective” is meant that the composition acts on the tissue whose size is to be reduced while having no effect, or significantly less effect, on at least one other tissue type, e.g., one, several, or many other tissue types (i.e., nontarget tissue types). By “tissue-specific” is meant that the composition acts on the tissue whose size is to be reduced while having no effect, or significantly less effect, on most or all other tissue types (i.e., nontarget tissue types).

In certain embodiments of the invention an effective composition reduces at least one dimension or, preferably, the volume of the target tissue, or an organ in which the target tissue is present, to between 0% and 95% of its initial value, e.g., to 5% or less, 10% or less, 20% or less, 30% or less, 40% or less, 50% or less, 60% or less, 70% or less, 80% or less, or 90% or less, or 95% or less of its initial value. Preferably an effective composition reduces the volume of a target tissue or organ in which the target tissue is present to 75% or less of its initial volume. Typically a reduction in volume will be accompanied by a reduction in wet and/or dry weight of the target tissue, organ, etc. The reduction in weight may be greater than, less than, or approximately the same as the reduction in volume on a percentage basis.

The reduction in dimensional size, volume, or weight can be expressed in terms of an initial size (Sᵢ), volume (Vᵢ), or weight (Wᵢ) and a final size (Fᵢ), volume (V_{f}), or weight (W_{f}). For purposes of description it will be assumed that the relevant parameter is volume, but the same considerations apply to size as determined by the value of one or more dimensions of the tissue or organ. The dimension can be, e.g., length, width, depth, diameter, or distance between any two points on a two-dimensional projection of the tissue or organ. In certain embodiments of the invention an effective composition results in a volume change such that V_{f}/Vᵢ≦0.95, V_{f}/Vᵢ≦0.90, V_{f}/Vᵢ≦0.80, V_{f}/Vᵢ≦0.70, V_{f}/Vᵢ≦0.60, V_{f}/Vᵢ≦0.50, V_{f}/Vᵢ≦0.40, V_{f}/Vᵢ≦0.30, V_{f}/Vᵢ≦0.20, V_{f}/Vᵢ≦0.10, V_{f}/Vᵢ≦0.05, or V_{f}/Vᵢ=0. A tissue-selective or tissue-specific composition may cause some reduction in the volume of a nontarget tissue, but the magnitude of the reduction is less. For example, Iin certain embodiments of the invention the value of V_{f}/Vᵢ for the nontarget tissue is at least 1.5 times as great as the V_{f}/Vᵢ for the target tissue, preferably at least 2 times as great. In certain embodiments of the invention the value of V_{f}/Vᵢ for the nontarget tissue is at least 3, 4, 5, 6, 7, 8, 9, 10, 20, 50, 100, or even more times as great as the V_{f}/Vᵢ for the target tissue. In certain embodiments of the invention the % reduction in volume of a nontarget tissue or organ is less than half the % reduction in volume of a target tissue or organ.

In general, a gene whose expression is regulated by an operably linked cell type specific regulatory element is considered to be tissue-specific for tissues that comprise cells of that type. Therefore, a gene that encodes a therapeutic nucleic acid or polypeptide whose expression is regulated by an operably linked cell type specific regulatory element such as a cell type specific promoter or enhancer is considered to be a tissue-specific therapeutic agent.

Tissue selectivity and/or specificity may be conferred by at least four different approaches, one or more of which is used in each of the various embodiments of the invention. One such approach is the use of a cell type specific therapeutic agent such as a gene whose expression is regulated by an operably linked cell type specific regulatory element such that the gene is specifically expressed in a cell type found in the tissue, preferably a cell type that is at least in part responsible for the tissue hypertrophy. The therapeutic agent selectively reduces cell division and/or kills the cell. A second approach is the use of local delivery. A third approach is the use of a cell type selective or cell type specific delivery vehicle. A delivery vehicle is an agent that is typically not itself effective by itself to reduce the size of the tissue but that is present within a therapeutic composition and serves one or more of of the following purposes. A delivery vehicle may enhance delivery of the therapeutic agent to cells or to a site within the body, e.g., by enhancing cell uptake or appropriate distribution of the therapeutic agent inside cells. A delivery vehicle may control or modulate bioavailability of the therapeutic agent, e.g., bioavailability may be controlled or modulated by the time course of release of the therapeutic agent from the vehicle. A delivery vehicle may stabilize the therapeutic agent (e.g., protect it from degradation), inhibit its uptake by nontarget cells (e.g., macrophages), inhibit its excretion, etc. A cell type selective or cell type specific delivery vehicle preferably selectively enhances delivery of the therapeutic agent to cells or tissues of particular type(s), selectively stabilizes the therapeutic agent in cells or tissues of particular type(s), and/or selectively controls or modulates release or distribution of the therapeutic agent within cells or tissues of particular type(s). A delivery vehicle is therefore distinct from commonly used pharmaceutical ingredients such as diluents or excipients that serve as bulking agents or fillers. A fourth approach, related to the third approach, is to use a delivery vehicle that is specifically targeted to a cell type of interest, e.g., a cell type that is prevalent within the tissue whose size is to be reduced. In certain embodiments of the invention at least two of these approaches for achieving tissue selectivity are used. In other embodiments of the invention at least three approaches for achieving tissue selectivity are used. In certain embodiments all four approaches are employed.

While the compositions and methods of the invention are of use in treating a wide variety of diseases and conditions associated with excessive or inappropriate tissue growth, one application of particular interest is the treatment of benign prostatic hyperplasia (BPH), sometimes referred to as benign prostatic hypertrophy. BPH will be taken as a representative context in which to describe certain of the inventive compositions and methods for treatment of hypertrophic tissues. The following section provides information on BPH, following which embodiments of the invention that employ each of the four approaches outlined above is discussed with particular reference to treatment of BPH.

III. Benign Prostatic Hyperplasia

The normal prostate gland weighs approximately 18 g, measures about 3 cm in length, 4 cm in width, and 2 cm in depth, and consists of approximately 70% glandular elements and 30% fibromuscular stroma. The prostate surrounds the prostatic urethra, into which the glandular secretions pass. The glands are lined with luminal epithelial cells, beneath which are basal epithelial cells that are believed to be stem cells for the secretory epithelium. Significant numbers of neuroendocrine cells that secrete a variety of hormonal polypeptides or biogenic amines are found throughout the gland.

The glandular components of the prostate can be divided into distinct zones, which can be distinguished, e.g., using ultrasonagraphy. The zones differ with respect to the location of their ducts relative to the urethra and the type of pathological lesions to which they are subject. The transition zone constitutes about 5%-10% of the glandular tissue and is the zone in which BPH most commonly arises. The central zone accounts for about 25% of the glandular tissue while the peripheral zone makes up the remainder (about 70%). In addition to the glandular and fibromuscular components, the prostate is supplied with blood vessels and nerves. Although much of the prostate is enclosed by a capsule consisting of collagen, elastin, and smooth muscle, the apex of the prostate, located inferiorly, is continuous with the striated muscle of the urethral sphincter, and normal prostatic glands can be found extending into the striated muscle, with no capsule or fibromuscular stroma separating them.

BPH is characterized by an increased number of cells in the periurethral region and/or transition zone of the prostate gland. The underlying mechanisms giving rise to this increase remain unclear. BPH may result from increased cell proliferation, decreased cell death (e.g., decreased apoptosis), or both. Both smooth muscle and epithelial (glandular) components typically exhibit an increase in cell number. A histologic diagnosis of BPH is typically based on the presence of stromoglandular hyperplasia on a biopsy, surgical, or autospy specimen, without evidence of cancer.

BPH causes or contributes to lower urinary tract symptoms (LUTS) in a significant proportion of aging men, most likely through a pathophysiological mechanism in which hyperplasia causes increased urethral resistance (obstruction), which in turn leads to changes in bladder muscle function. LUTS include urinary frequency, urgency, and nocturia. Other related symptoms include hesitancy, straining, dribbling, intermittency, incomplete emptying, weak stream, dysuria, irritability, and wet clothes. Patients experiencing one or more of these symptoms may be classified by their level, e.g., into mildly, moderately, or severely smptomatic. A standardized questionnaire such as the American Urological Association (AUA) Symptom Index, also known as the International Prostate Symptom Score (IPSS) may be used (18, 19). The total score ranges between 0 and 35. Patients scoring between 1 and 7 may be classified as mildly symptomatic, those scoring between 8 and 19 as moderately symptomatic, those scoring between 20 and 35 as severely symptomatic. These ranges are exemplary only, and other ranges, or other standardized questionnairs, could also be used.

Patients may also be classified according to the degree of prostatic enlargement, which can be measured by digital rectal examination (DRE), transrectal ultrasonography (TRUS), magnetic resonance imaging (MRI), etc. For example, a prostate gland may be considered enlarged if it has a volume greater than about 20 ml, greater than about 25 ml, greater than about 30 ml, etc.

Patients may also be classified by the degree of outlet obstruction, which can be measured by flow rate recordings or pressure flow studies. For example, a maximum flow rate of less than 10 ml/sec indicates a high likelihood of obstruction. A maximum flow rate of between 10 and 15 ml/sec is also indicative of obstruction according to certain diagnostic criteria.

More severe consequences or complications of BPH can include bladder stones, urinary tract infections, bladder decompensation, urinary incontinence, renal failure, hematuria, and acute urinary retention.

The compositions and methods of the present invention may be used for treatment of BPH as diagnosed based on the presense of one or more symptoms of LUTS, e.g., of mild, moderate, or severe LUTS, prostatic enlargement, outflow obstruction, the existence of one or more of the severe complications mentioned above, histopathologic evidence of BPH, or any combination of the foregoing. A decision to initiate treatment of BPH using a composition and/or method of the invention may be based on an initial diagnosis, e.g., identification of one or more symptoms of LUTS, etc., or may be based on a worsening of BPH as evidenced by an increase in severity of LUTS, an increase in prostate volume, a decrease in maximum flow rate, the emergence of a severe complication of BPH, the failure of a patient's current therapy to achieve acceptable results, increased histopathologic evidence of BPH, or any combination of the foregoing.

IV. Tissue-Selective and Tissue-Specific Therapy

As mentioned above, the invention provides compositions and methods for tissue-selective and/or tissue-specific therapy of a disease or condition characterized by inappropriate or excessive noncancerous tissue growth. Tissue selectivity or specificity is achieved using a cell type specific therapeutic agent, local delivery, a cell type selective delivery vehicle, a targeted delivery vehicle, or any combination of the foregoing. Each of these approaches is further discussed below.

A. Cell Type Specific Therapeutic Agents

Cell type specific therapeutic agents are active only or primarily in a cell type that is present in the tissue that is to be reduced in volume and/or whose activity is higher in a cell type present in the tissue that is to be reduced in volume than in many or most other cell types. Preferably the cell type is prevalent in the tissue whose volume is to be reduced, e.g., represents at least 10%, more preferably at least 20%, 30%, 40%, 50%, 60%, 70%, 80%, 90% or more of the cells and/or volume of the tissue that is to be reduced. For example, the cell type may constitute a significant fraction (at least 20%), a substantial fraction (at least 50%), or a major fraction (at least 80%) of the cells in the tissue whose volume is to be reduced. In certain embodiments of the invention the cell type for which the therapeutic agent is specific is a cell type that is at least in part directly responsible for causing the inappropriate or excessive tissue growth, i.e., proliferation and/or hypertrophy cells of cells of that cell type is occurring. The cell type may be at least in part indirectly responsible for causing the inappropriate or excessive tissue growth, e.g., by secreting a molecule that induces cell proliferation or hypertrophy of another cell type. The cell type specific therapeutic agent may be active in more than one cell type present in the tissue, in which case preferably the cell types collectively are prevalent in the tissue whose volume is to be reduced and/or are at least in part directly or indirectly responsible for causing the inappropriate or excessive tissue growth. For example, a preferred cell type specific therapeutic agent for treatment of BPH is active in prostate gland epithelial cells (e.g., luminal cells, basal cells, or both), prostate gland smooth muscle cells, or both.

Suitable cell type specific therapeutic agents include vectors in which a nucleic acid that encodes a therapeutic polypeptide (e.g., a cytotoxic or cytostatic peptide) or that provides a template for transcription of a therapeutic nucleic acid is operably linked to a cell type specific regulatory element so that the therapeutic nucleic acid or polypeptide is produced specifically in a target cell type or types. The regulatory element may comprise a cell type specific promoter, a cell type specific enhancer, a cell type specific combined promoter/enhancer, or modified versions of any of the foregoing.

The vector may comprise multiple regulatory elements, not all of which need be cell type specific. For example, the vector may comprise a ubiquitous regulatory element, e.g, a promoter, that displays a basal level of activity in a variety of different cell types and a cell type specific regulatory element such as an enhancer that increases the level of activity of the promoter in a cell type of interest, e.g., a cell type within a tissue whose size is to be reduced. Either regulatory element can be partly or entirely synthetic (i.e., not found in nature) and may contain multiple copies of one or more domains found in a naturally occurring regulatory element. A ubiquitous regulatory element is a regulatory element, e.g., a promoter, that is active in most or all cell types under normal physiological conditions and preferably displays strong activity in most or all cell types. A constitutive regulatory element is active in one or more cell types under normal physiological conditions and/or is not subject to regulation by a particular inducing agent or environmental condition. Constitutive regulatory elements may, but need not be, ubiquitous.

A large number of ubiquitous and constitutive regulatory elements are known in the art. The web site having URL www.invivogen.com/plasmids/promoters_{—}2.htm provides a list that includes a variety of native and composite ubiquitous and cell type specific regulatory elements (referred to collectively as promoters on that web site) that direct transcription in primate and/or rodent cells. Composite regulatory elements contain components taken from different naturally occurring regulatory regions either from the same or different genes (e.g., an enhancer and a promoter), which are combined to create a composite regulatory element. The components may be modified in addition to or instead of being combined with one another. Multiple copies of one or more regulatory elements may be included. A number of these regulatory elements are available in the pDRIVE series of plasmids desribed at the web site having URL www.invivogen.com/plasmids/promoters.htm.

Examples of ubiquitous regulatory elements include the Rous sarcoma virus (RSV) or cytomegalovirus (CMV) promoter or promoter/enhancer (e.g., CMV early promoter), or a modified version such as the CAG promoter/enhancer. CAG is a composite regulatory element that combines the human cytomegalovirus immediate-early enhancer and a modified chicken beta-actin promoter and first intron (24). The CAG promoter is a very strong and ubiquitous promoter that produces high levels of expression both in vitro and in vivo and has been successfully used to express enhanced GFP in all tissues of transgenic mice with the exception of erythrocytes and hair (25). Comparison analyses have shown that the CAG promoter is more efficient than the CMV promoter/enhancer (26). Additional examples include regulatory elements such as promoters for housekeeping genes, e.g., elongation factor 1α (27, 28), phosophoglycerate kinase-1 (29, 30), beta-actin (31, 32), ubiquitin B (33), ubiquitin C (34), etc. Beta-actin is a highly conserved protein ubiquitously expressed in all eukaryotic cells. A 1.2-kb fragment of the human β-actin 5′ flanking region is sufficient for efficient transcription. A 1.1 kb fragment from the ubiquitin B gene was shown to display sustained expression of a transgene in vitro and in vivo. In certain embodiments of the invention a regulatory element such as a CMV or CAG promoter/enhancer that is capable of directing transcription in a variety of different cell types (e.g., rodent, primate, canine, etc.) is used. Preferably the regulatory element directs transcription in human cells.

A large number of genes are known in the art to be expressed in a cell type specific manner. Naturally occurring regulatory elements that control expression of these genes, or regulatory elements derived from naturally occurring regulatory elements that control expression of these genes, can be used to direct cell type specific expression of a therapeutic polynucleotide or polypeptide. Of particular relevance for the present invention are regulatory elements derived from genes that are expressed in a cell type specific manner in cells that are present in noncancerous tissues in which inappropriate or excessive noncancerous tissue growth may occur. Such tissues include, but are not limited to, prostate tissue, thyroid tissue, adipose tissue, breast tissue, fibromuscular tissues, fibrous tissues, etc. In certain embodiments of the invention the regulatory elements are derived from genes that are expressed in specifically expressed in cancerous as well as noncancerous tissue.

A number of genes are expressed in a tissue-specific manner in the noncancerous prostate gland, i.e., they are specifically expressed in one or more cell types in the noncancerous prostate gland (and, in some cases, also in prostate cancer cells). Among these are the genes that encode prostate specific antigen (PSA), kallikrein 2 (hK2-protein; KLK2-gene), prostate specific membrane antigen (PSMA), probasin, prostate stem cell antigen (PSCA), prostate secretory protein of 94 amino acids (PSP94), and T cell receptor gamma-chain alternate reading frame protein (TARP). PSA is a glycoprotein with a molecular weight of about 33 kD that acts as a serine protease and is found almost exclusively in prostate gland epithelial cells as well as prostate secretions and serum. PSMA is a membrane-bound glycoprotein also found almost exclusivly in prostate gland epithelial cells. Kallikrein 2 is a prostate-specific serine protease closely related to PSA. PSCA is a cell surface antigen expressed in a subset of prostate gland epithelial cells that have not yet terminally differentiated to a secretory phenotype. Probasin and PSP94 are among the most abundant proteins secreted from the human prostate and are generally considered to be prostate tissue-specific in both human and rodents. TARP is a protein that in males is uniquely expressed in prostate epithelial cells.

Regulatory elements of these genes have been identified. For example, promoter and enhancer regions of the PSA gene are known and have been combined to produce regulatory elements that direct higher levels of expression than the native PSA regulatory region while still retaining cell type specificity (14). A 6 kB region lying largely upstream of the PSA coding sequence contains sufficient genetic information to direct prostate-specific expression in the mouse. The PSA regulatory region includes an enhancer core, which contains sites for androgen binding known as androgen responsive elements (ARE), and a proximal promoter (14, and references therein). Composite PSA regulatory regions containing multiple AREs, multiple enhancer cores, and/or removal of intervening sequences between the enhancer and promoter demonstrated increased activity relative to wild type PSA regulatory sequences. Specific composite PSA regulatory regions referred to as PSE-BA, PSE-BC, and PSA-BAC have been described (14). Additional composite PSA regulatory regions showing enhanced expression relative to the unmodified PSA regulatory region were obtained by similar strategies (35). PSE-BC is a chimeric modified enhancer/promoter sequence of the human prostate-specific antigen (PSA) gene. This promoter sequence is active discriminately in luminal cells in the mouse prostate, thus reflecting its activity in PSA-expressing cells in human prostate (107, 108).

Unmodified and composite regulatory elements, e.g., promoters, enhancers, and promoter/enhancer regions derived from the regulatory regions of the PSMA (36-38), probasin (39), PSP94 (40), TARP (41), and PSCA (42) genes have been identified or created and shown to be prostate-specific. Some of the composite regulatory elements comprise genetic components obtained from different prostate-specific genes. Thus a number of prostate-specific regulatory elements that could be used to direct expression of a therapeutic polynucleotide or polypeptide in a prostate-specific manner in accordance with the present invention are known.

Various genes are known to be selectively expressed in different cell types found in the prostate gland. For example, epithelial cells express cytokeratins 8 and 13; stromal cells express vimentin, but not cytokeratins, and smooth muscle cells express β-actin. Regulatory elements from these genes could be used to direct expression in a tissue-selective manner.

A number of genes are expressed in a tissue-specific manner in the noncancerous thyroid gland, i.e., they are specifically expressed in one or more cell types in the noncancerous thyroid gland (and, in some cases, in cancerous thyroid tissue). Among these are genes encoding thyroglobulin (TG), calcitonin (CALC), Pax-8, thyroperoxidase (TPO), thyrotropin receptor (TSH-R) and the sodium/iodide symporter (NIS). Regulatory regions of a number of these genes that direct tissue-specific expression have been identified. Promoters, enhancers, and composite regulatory elements combining one or more copies of certain promoters and/or enhancers have been identified or created (43-46). Thus a number of thyroid-specific regulatory elements that could be used to direct expression of a therapeutic polynucleotide or polypeptide in a thyroid-specific manner for treatment of hyperthyroidism in accordance with the present invention are known. Activity of certain of these regulatory elements is enhanced by treatment with agents that modulate the cAMP pathway, such as 8-Br-cAMP, and histone deacetylase inhibitors such as depsipeptide (43). In certain embodiments of the invention a therapeutic composition comprises one or more of these compounds.

Genes and proteins that are differentially expressed in adipose cells and adipose tissues of various types, or in adipose tissue in obese versus nonobese subjects have been identified (47, 48). Adiponectin or adipocyte complement-related protein of 30 kDa (Acrp30) is a circulating protein produced exclusively in adipocytes (49). Desnutrin is predominantly expressed in adipose tissue and its expression is induced early during 3T3-L1 adipocyte differentiation (50). Asb6 is an adipocyte-specific ankyrin and SOCS box protein (51). Regulatory regions of these genes can be used to direct cell type specific expression of a therapeutic nucleic acid or polypeptide in adipose tissue for treatment of obesity or reduction in undesired adipose tissue in accordance with the present invention.

Cytokeratin 5/6 regulatory elements can be used to direct expression in breast tissue. Regulatory elements mentioned above can be used to direct expression to adipose tissue in the breast.

Keratins are intermediate filament proteins that are components of the cytoskeleton in epithelial cells throughout the body. A large number of keratin genes have been identified, and their expression in epithelial cells of different types has been examined (52 and reference therein). Regulatory elements (e.g., promoters, enhancers, composite elements) derived from regulatory regions of keratins that are specifically expressed in epithelial cells of one or more types (e.g., keratinocytes) can be used to direct cell type specific expression of a therapeutic nucleic acid or polypeptide in tissues in which such cells are present for treatment of excessive or unwanted epithelial tissue growth (e.g., scars).

Genes that are differentially expressed in benign tumors such as uterine leiomyoma (fibroids) have been identified (53). Regulatory elements (e.g., promoters, enhancers, composite elements) derived from regulatory regions of genes that are specifically expressed in cells (e.g., smooth muscle cells) in leiomyomas can be used to direct cell type specific expression of a therapeutic nucleic acid or polypeptide in leiomyomas. Genes that are specifically expressed in cells found in other benign tumors, or in other tissues in which excessive or unwanted growth may occur are known in the art and are accessible in the scientific literature to one of ordinary skill in the art. Regulatory elements from such genes can be used to direct cell type specific expression of a therapeutic nucleic acid or polypeptide in tissues in which such cells are present for treatment of excessive or unwanted tissue growth.

The invention is in no way limited to use of previously identified regulatory elements or to regulatory elements that have the precise boundaries of regulatory elements that have been described in the art and/or herein. One of ordinary skill in the art will appreciate that often a variety of segments of different lengths that contain a particular region of genomic DNA will serve as a tissue-specific regulatory element. If desired, the precise minimal boundaries required to achieve a desired tissue specificity can be identified by examining the ability of a panel of deletion derivatives of a segment (or segments) of DNA that contains a tissue-specific regulatory region to direct tissue specific expression. However, typically larger segments of DNA containing the minimal regulatory region will also be of use.

The various polypeptides of interest discussed herein are referred to by their common names as understood by one of ordinary skill in the art. Sequence information is readily available for each of these proteins, e.g., in public databases such as GenBank. One of ordinary skill in the art will be able to identify the appropriate protein and corresponding nucleic acid sequences for any particular species of interest using the relevant scientific literature and databases. It is noted that frequently a number of entries for each protein appear. Many genes have been assigned a unique identifier known as a Gene ID. Multiple entries and references to the gene are collected under the Gene ID. As known to one of ordinary skill in the art, Gene IDs can be found using Pubmed at the National Center for Biotechnology Information (NCBI), as can GenBank accession numbers. The website has URL www.pubmed.com. The Gene ID search is performed by selecting “Gene” from the pull-down menu at the top left (below “nucleotide”, “protein”, etc.). The following list provides Gene IDs for the human forms of a number of the genes mentioned herein that are expressed by one or more cell types found in the prostate gland.

PSA: 354

TARP: 445347

KLK2: 3817

PSMA: 2346

PSCA: 8000

PSP94: 4477

In certain embodiments of the invention cell type specificity is achieved using a vector in which expression of a gene that encodes a therapeutic polynucleotide or polypeptide is controlled both by transcriptional regulation and regulated recombination. In some embodiments the vector contains a coding sequence for a recombinase, e.g., a site-specific recombinase, which is placed under control of a cell type specific regulatory element such that transcription of the recombinase occurs at significant levels only in a desired cell type or types, e.g., in a target cell type or types. Recombination catalyzed by the recombinase preferably results in excision of sequences located between two specific sites for recombination.

Any of a number of site-specific recombinase systems known in the art can be used (20, 21). For example, the Cre/loxp (22) or Flp/FRT system (23) can be used. The recombinase can be a monomer, dimer, heterodimer, multimer, or heteromultimer. In embodiments in which the recombinase comprises two or more subunits, the expression of at least one of the subunits is under control of a cell type specific regulatory element.

The vector also contains (i) a nucleic acid that encodes a therapeutic polynucleotide or polypeptide and (ii) a second regulatory element that includes a promoter capable of driving transcription in the cell type of interest and (iii) optionally includes additional sequences, e.g., enhancer sequences. However, the nucleic acid is not operably linked to the second regulatory element but instead is separated from it by a region that includes target sites for recombination by the site-specific recombinase, such that recombination brings the nucleic acid into operable association with the second regulatory element so that transcription of the nucleic acid occurs. It will be appreciated that recombinases whose activity results in inversion of a nucleic acid sequence without necessarily involving removal of all or part of the sequence can also be used, in which case inversion brings the nucleic acid that encodes the therapeutic agent into operable association with the second regulatory element.

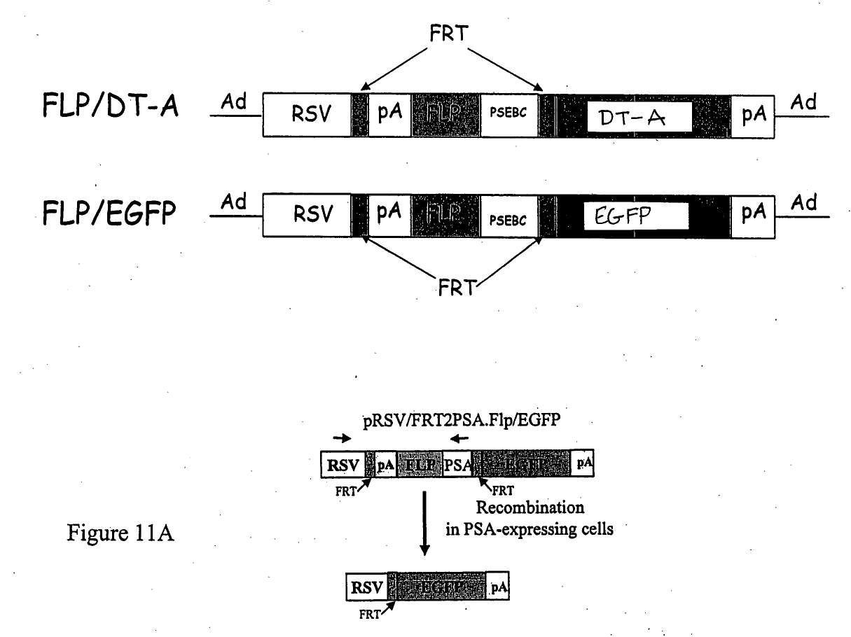

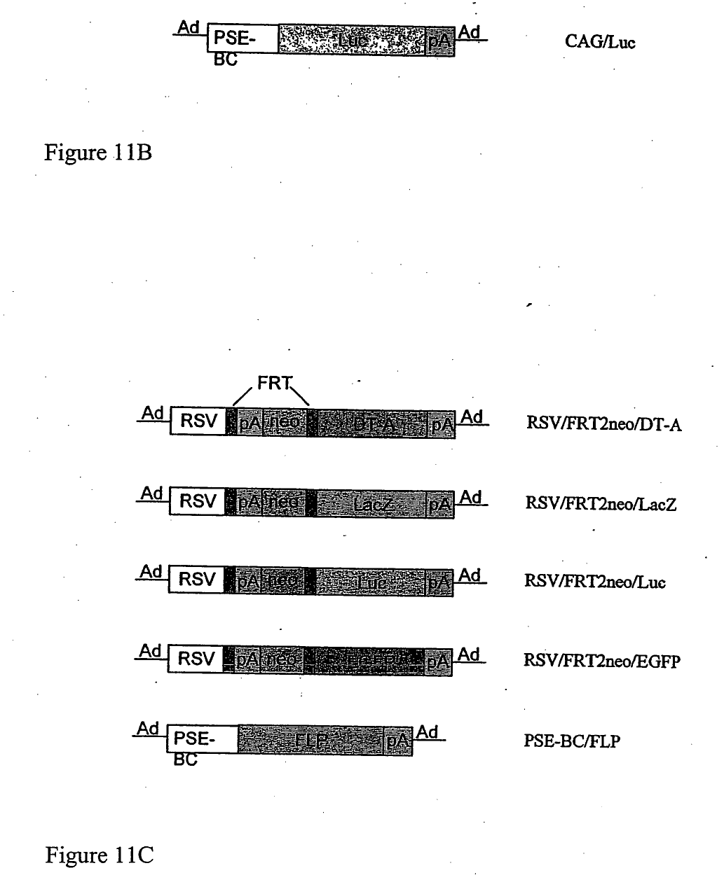

FIG. 11A show examples of nucleic acid constructs containing an arrangement of elements for controlling expression of a polypeptide by transcriptional regulation and regulated recombination. The site-specific recombinase is Flp, which catalyzes excision of DNA located between sites referred to as FRT. The nucleic acid encoding Flp is operably linked to a regulatory element specific for prostate gland cells, i.e., PSE-BC, which is discussed further below (14). The second regulatory element is the RSV promoter. Recombination removes the sequences between the FRT sites, bringing the RSV promoter into operable association with a nucleic acid that encodes diphtheria toxin A chain (DT-A) or enhanced green fluorescent protein (EGFP), as shown for EGFP (FIG. 11A, bottom), where PSA represents the prostate-specific PSE-BC promoter/enhancer element. See also U.S. Ser. No. 60/550,912, PCT/US05/007001, and U.S. Ser. No. 11/074,323, which describe similar nucleic acid constructs that may be used in the present invention. The construct is inserted into a suitable vector, e.g., a plasmid or recombinant viral genome. The vector (in its unrecombined state) is introduced into cells in culture or into a subject. Recombination occurs in cells in which the cell type specific regulatory element is active, e.g., prostate gland cells in the case of a regulatory element that is specifically active in prostate gland cells. Thus transcription of mRNA encoding DT-A occurs in these cells.

It will be appreciated that a variety of other arrangements could be used. For example, in FIG. 11A transcription of Flp proceeds from right to left. However, the arrangement of PSE-BC and Flp coding sequences could be reversed, in which case transcription would proceed from left to right. Genetic elements such as polyA sites (pA), transcriptional terminators, ribosome binding sites, internal ribosome entry sites, locus control regions, 5′ or 3′ untranslated regions, matrix attachment regions, etc., may be included (92). The various coding sequences and other genetic elements could be present on two separate nucleic acid constructs, e.g., as shown in FIG. 11C.

Instead of being a ubiquitous or constitutive promoter, the second regulatory element may instead be a cell type specific regulatory element capable of driving transcription in the desired cell type(s), e.g., target cell type(s). In certain embodiments of the invention a single regulatory element is used to achieve both transcriptional regulation and regulated recombination. Prior to recombinase-mediated recombination such a vector contains a cell type specific regulatory element in operable association with a nucleic acid that encodes a site-specific recombinase. The sequence that encodes the site-specific recombinase is located between the regulatory element and the nucleic acid that encodes the therapeutic polynucleotide or polypeptide and is flanked by sites for site-specific recombination. Recombination brings the regulatory element into operable association with the nucleic acid that encodes the therapeutic polynucleotide or polypeptide.

In other embodiments of the invention both transcriptional regulation and regulated recombination are achieved by using a construct containing a nucleic acid that encodes a fusion protein comprising a ligand-responsive domain fused to a site-specific recombinase. Administration of the ligand activates the fusion protein in any of a number of ways. For example, administration of the ligand may cause a conformational change that allows the recombinase to become active, causes the fusion protein to translocate into the nucleus, etc. In certain embodiments of the invention the ligand-responsive domain is a hormone binding domain such as an estrogen-binding domain. Adminstration of estrogen or an analog such as tamoxifen causes translocation of the fusion protein into the nucleus, where it catalyzes recombination of a construct containing sites for the recombinase (5).

The recombinase gene and the nucleic acid that encodes the therapeutic polynucleotide or polypeptide, together with the respective regulatory elements with which the gene and nucleic acid are or become operably linked can be part of the same nucleic acid or different nucleic acids. If either the recombinase or therapeutic polynucleotide or polypeptide comprises multiple subunits these can be encoded by one or more nucleic acid constructs, which can be present in one or more vectors.

Cell type specific therapeutic agents may be delivered either systemically or locally and still result in tissue-selective or tissue-specific effects. Both delivery methods are discussed further below.

B. Delivery Vehicles and Cell Type Selective Delivery

A therapeutic composition may comprise a variety of different delivery vehicles. In certain embodiments of the invention a nonviral delivery vehicle is used. While viral delivery systems are often efficient means of delivering nucleic acids to cells, nonviral nucleic acid delivery systems can offer a number of advantages including stability, cost and ease of production, low immunogenicity and toxicity, and ability to deliver larger nucleic acids (69, 70). Nonviral delivery vehicles may, of course, also be used to deliver agents other than nucleic acids including, but not limited to, small molecules, proteins, etc. By “nonviral delivery vehicle” is meant any agent that does not utilize a virus or viral capsid as a mechanism to achieve entry of a therapeutic agent into cells. Viruses and viral capsids are considered to be viral delivery vehicles. A nonviral delivery vehicle may, in certain embodiments of the invention, comprise one or more viral proteins or portion(s) thereof and/or one or more viral nucleic acids or portion(s) thereof.

In certain embodiments of the invention the therapeutic composition comprises a biocompatible polymer, which preferably is biodegradable. Suitable polymers include, but are not limited to, poly(lactic-co-glycolic acid), polyanhydrides, ethylene vinyl acetate, polyglycolic acid, chitosan, polyorthoesters, polyethers, polylactic acid, and poly (beta amino esters). Peptides, proteins such as collagen, and dendrimers (e.g., PAMAM dendrimers) can also be used.

The inventors have described a class of polymers referred to as poly (beta amino esters) that show particular promise as delivery agents, as they are highly efficient in vitro, and easily synthesized via the conjugate addition of a primary amine or bis(secondary amine) to a diacrylate. These compounds are described in detail in U.S. provisional patent applications 60/239,330, filed Oct. 10, 2000 and 60/305,337, filed Jul. 13, 2001, in U.S. patent applications 09/969,431, filed Oct. 2, 2001, and 10/446,444, filed May 28, 2003 (publication number 20040071654, and in references (10, 11, 71, 72). In certain embodiments of the invention a poly (beta amino ester) compound, or a salt or derivative thereof, is used as a delivery vehicle. The compound can be used in the form of microparticles, nanoparticles, solid drug delivery articles, and/or as a soluble nanometer scale complex with a nucleic acid.

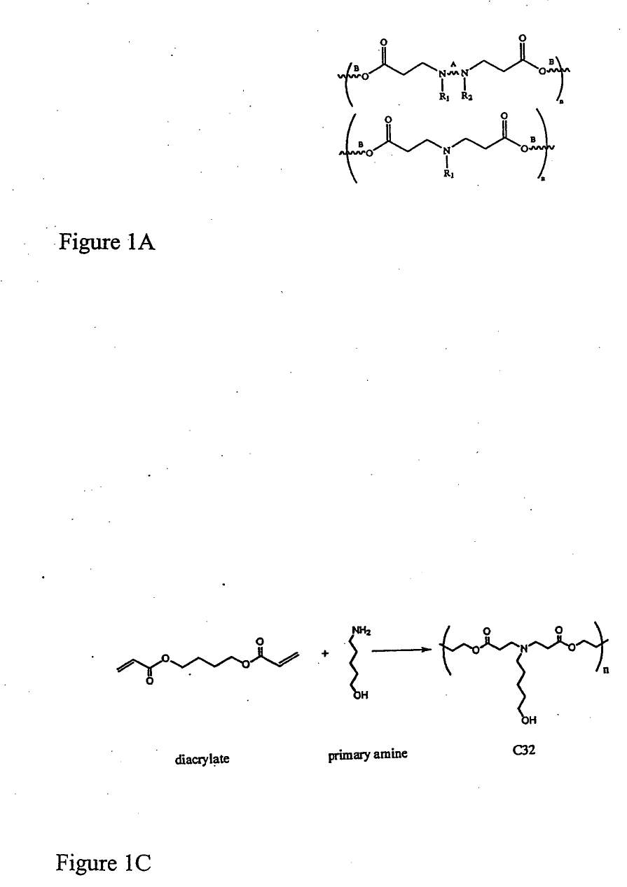

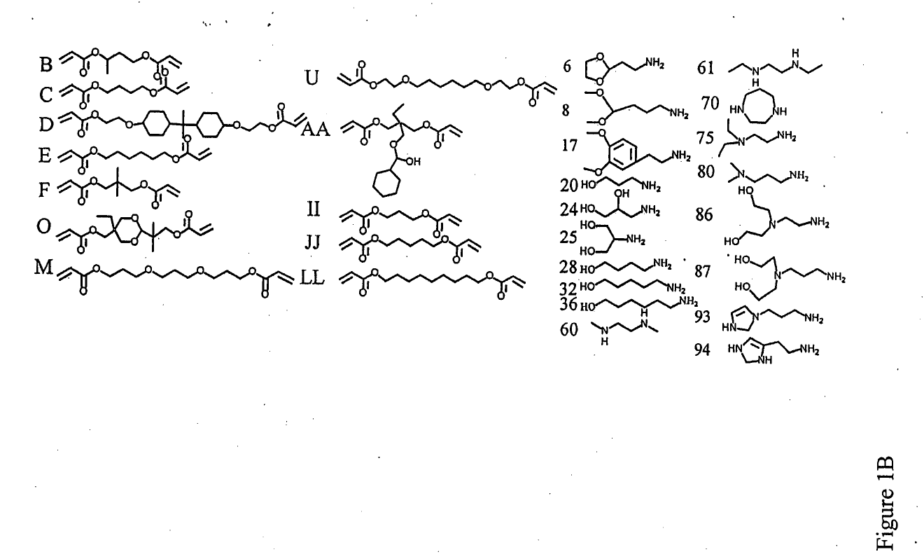

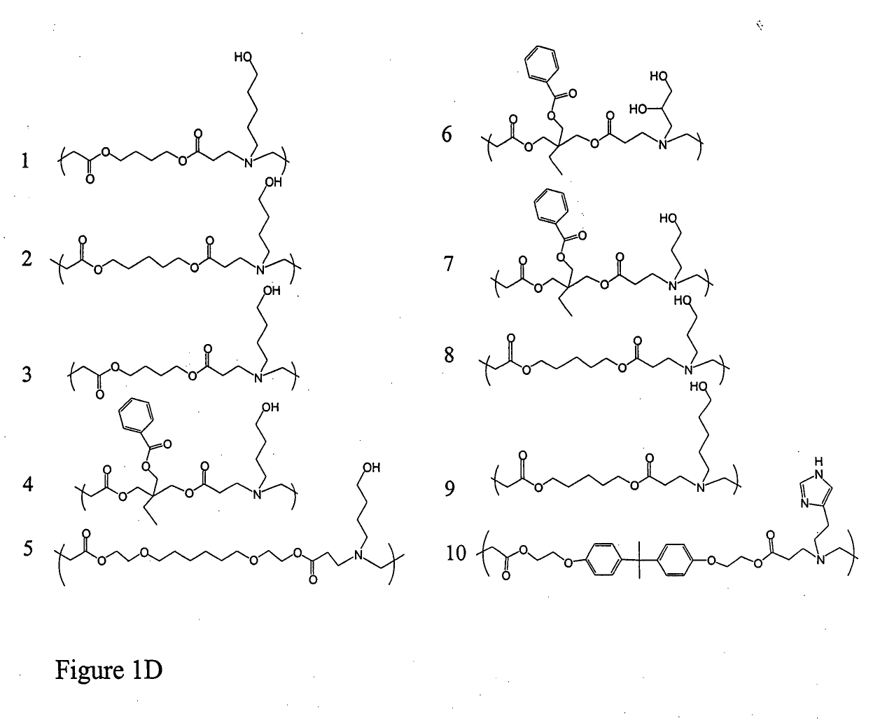

The poly (beta amino ester) compounds are generally represented by formulas 1 and 2 in FIG. 1A. The compounds may be formed by condensing bis(secondary amines) or primary amines with bis(acrylate esters). FIG. 1B shows structures of a variety of different acrylate and amine monomers that can be condensed to form a poly (beta amino ester). Additional monomers are described in U.S. Ser. No. 10/446,444. In certain embodiments of the invention a poly (beta amino ester) comprising an acrylate selected from structures B, C, D, E, F, O, M, U, AA, II, JJ, or LL as shown in FIG. 1B is used as a delivery vehicle. In certain embodiments of the invention a poly (beta amino ester) comprising an amine selected from structures 6, 8, 17, 20, 24, 25, 28, 32, 36, 60, 61, 70, 75, 80, 86, 87, 93, or 94 as shown in FIG. 1B is used as a delivery vehicle. The polymers are named using a letter to represent an acrylate and a number to represent an amine. C32, JJ28, and U28 are representative examples of monomers that may be used. In general, the polymers described herein contain n monomers, wherein n is between 3 and 10,000, inclusive. FIG. 1C shows the structure of a monomer of C32, a polymer with a particularly high ability to transfect cells with DNA. In certain embodiments of the invention a polymer comprising monomers having a formula selected from the group consisting of formulas 1-10 (FIG. 1D) below, or a derivative or salt thereof, is used, wherein n is an integer between 3 and 10,000.

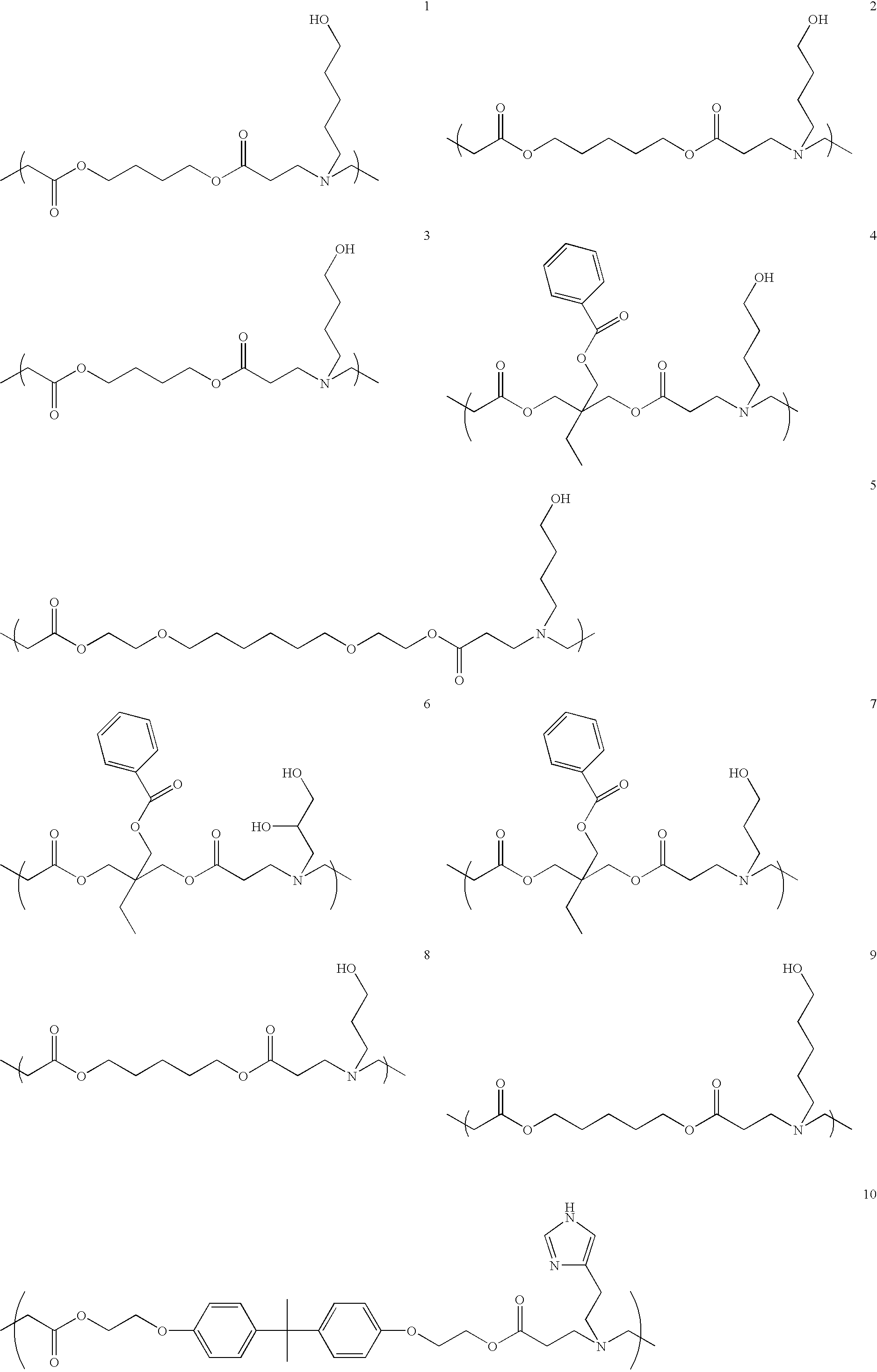

The poly (beta amino ester) may be synthesized using an acrylate:amine ratio of greater than 1:1, e.g., between 1.05:1 and 1.5 to 1 and may be amine-terminated at one or both ends. Preferably the poly (beta amino ester) condenses DNA and/or RNA to form soluble nanoparticles 500 nm or less in diameter, e.g., 50-500 nm in diameter, 50-100 nm in diameter, etc. The poly (beta amino ester) may have a positive zeta potential, e.g., a zeta potential between 1 and 30 mV, between 5 and 10 mV, between 10 and 15 mV, between 10 and 20 mV, etc. The average molecular weight of the polymer may be at least approximately 5 kD, preferably at least approximately 10 kD, e.g., 10-15 kD, 10-20 kD, 20-30 kD, etc.

In certain embodiments of the invention the composition may be a drug delivery device comprising a solid material such as polymeric matrix impregnated with, or encapsulating, a therapeutic agent. The device is implanted into the body at the location of the target tissue or in the vicinity thereof, or in a location distant from the target tissue. The therapeutic agent is typically released from the polymeric matrix over a period of time, e.g. by diffusion out of the matrix or release into the extracellular environment as the matrix degrades or erodes. If the device is implanted at a location distant from the target tissue, e.g., too far for effective concentrations of agent to reach the target tissue by diffusion, the therapeutic agent may be transported to the target tissue in the blood.

A polymeric matrix comprising the therapeutic agent may assume a number of different shapes. For example, microparticles of various sizes (which may also be referred to as beads, microbeads, microspheres, nanoparticles, nanobeads, nanospheres, etc.) can be used. Polymeric microparticles and their use for drug delivery are well known in the art. Such particles are tyically approximately spherical in shape but may have irregular shapes. Generally, a microparticle will have a diameter of 500 microns or less, e.g., between 50 and 500 microns, between 20 and 50 microns, between 1 and 20 microns, between 1 and 10 microns, and a nanoparticle will have a diameter of less than 1 micron. If the shape of the particle is irregular, then the volume will typically correspond to that of microspheres or nanspheres. The polymeric matrix can be formed into various nonparticulate shapes such as wafers, disks, rods, etc., which may have a range of different sizes and volumes. Methods for incorporating therapeutically active agents into polymeric matrices are known in the art.

Solid nanoparticles or microparticles can be made using any method known in the art including, but not limited to, spray drying, phase separation, single and double emulsion solvent evaporation, solvent extraction, and simple and complex coacervation. Preferred methods include spray drying and the double emulsion process. Solid agent-containing polymeric compositions can also be made using granulation, extrusion, and/or spheronization.

The conditions used in preparing the microparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the polymer matrix. If the particles prepared by any of the above methods have a size range outside of the desired range, the particles can be sized, for example, using a sieve.

Methods developed for making microparticles for delivery of encapsulated agents are described in the literature (64-67).

Solid nanoparticles or microparticles can be suspended or dispersed in a pharmaceutically acceptable fluid such as physiological saline and administered by injection at or near a site of tissue hypertrophy and/or into the bloodstream. They can also be administered by any of a number of other routes mentioned below.

Solid polymer-agent compositions (e.g., disks, wafers, tubes, sheets, rods, etc.) can be prepared using any of a variety of methods that are well known in the art. For example, in the case of polymers that have a melting point below the temperature at which the composition is to be delivered and/or at which the polymer degrades or becomes undesirably reactive, a polymer can be melted, mixed with the agent to be delivered, and then solidified by cooling. A solid article can be prepared by solvent casting, in which the polymer is dissolved in a solvent, and the agent is dissolved or dispersed in the polymer solution. Following evaporation of the solvent, the substance is left in the polymeric matrix. This approach generally requires that the polymer is soluble in organic solvent(s) and that the agent is soluble or dispersible in the solvent. In still other methods, a powder of the polymer is mixed with the agent and then compressed to form an implant.

Certain of the delivery vehicles mentioned above, and others, can be used for delivery of nucleic acids. In certain embodiments of the invention a polymer that forms a complex with a nucleic acid is used as a delivery vehicle. The polymer may form a complex with DNA, RNA, and/or modified DNA, RNA, etc. A variety of cationic polymers that form complexes with nucleic acids are known in the art. Cationic polymers are known to spontaneously bind to and condense nucleic acids such as DNA into nanoparticles. For example, naturally occurring proteins, peptides, or derivatives thereof have been used (76, 77). Synthetic cationic polymers such as polyethylenimine (PEI), polylysine (PLL), polyarginine (PLA), polyhistidine, etc., are also known to condense DNA and are useful delivery vehicles (78). References (103-105); U.S. Ser. No. 6,013,240; WO9602655 provide further information on PEI. Cationic polymers modified by addition of groups such as acyl, succinyl, acetyl, or imidazole groups, e.g., to reduce cytotoxicity, can be used. Dendrimers can also be used (75, 81).

Many of the useful polymers contain both chargeable amino groups, to allow for ionic interaction with the negatively charged DNA phosphate, and a degradable region, such as a hydrolyzable ester linkage. Examples of these include poly(alpha-(4-aminobutyl)-L-glycolic acid) (73), network poly(amino ester) (74), and poly (beta-amino esters) (10, 11, 71, 72, and patent applications mentioned above). These complexation agents can protect DNA against degradation, e.g., by nucleases, serum components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid (79). It has been proposed that such agents may act as a “proton sponge” within the endosome.

In certain embodiments of the invention a polymer/nucleic acid complex comprising a poly (beta amino ester) is used. In general, any poly (beta amino ester) described above may be used. The inventors have screened libraries of poly (beta amino ester) compounds to identify general properties and specific polymers that may be of particular use for delivery of nucleic acids to cells. See, e.g., Example 1. In certain preferred embodiments of the invention a poly (beta amino ester) comprising an acrylate monomer and an amine monomer selected from those pictured in FIG. 1A is used. For example, C32, JJ28, or C28 may be used.

The poly (beta amino ester)/nucleic acid complex may also contain one or more of the nucleic acid delivery vehicles mentioned above as a co-complexation agent. Co-complexing agents bind to polynucleotides and/or increase transfection efficiency. Co-complexing agents usually have a high nitrogen density. Polylysine (PLL) and polyethylenimine (PEI) are two examples of polymeric co-complexing agents. PLL has a molecular weight to nitrogen atom ratio of 65, and PEI has a molecular weight to nitrogen atom ratio of 43. Any polymer with a molecular weight to nitrogen atom ratio in the range of 10-100, preferably 25-75, more preferably 40-70, may be useful as a co-complexing agent. The inclusion of a co-complexing agent in a complex may allow one to reduce the amount of poly(beta-amino ester) in the complex. This becomes particularly important if the poly(beta-amino ester) is cytotoxic at higher concentrations. In the resulting complexes with co-complexing agents, the co-complexing agent to polynucleotide (w/w) ratio may range from 0 to 2.0, preferably from 0.1 to 1.2, more preferably from 0.1 to 0.6, and even more preferably from 0.1 to 0.4. In certain embodiments of the invention agents such as polyacrylic acid (pAA), poly aspartic acid, polyglutamic acid, or poly-maleic acid may be used to alter the charge of the complex, which may prevent serum inhibition of the polynucleotide/polymer complexes in cultured cells in media with serum (82) and/or in a subject.

Polymer/nucleic acid complexes may be formed by contacting the polymer and nucleic acid under any conditions suitable for formation of polymer/nucleic acid complexes. For example, a solution containing the nucleic acid may be added to a gently vortexing solution of a polymer or salt thereof. The solution may be at a pH of between about 4 and 9, more preferably between 5 and 8, e.g., about 6.0 to 7.5. Concentrations of polymer and DNA may be adjusted to achieve a desired polymer/nucleic acid ratio. For example, the weight to weight ratio of polynucleotide to polymer may range from 1:0.1 to 1:200, preferably from 1:10 to 1:150, more preferably from 1:50 to 1:150. The amine monomer to polynucleotide phosphate ratio may be approximately 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, 160:1, 170:1, 180:1, 190:1, and 200:1. In certain embodiments, the ratio of nitrogen in the polymer (N) to phosphate in the polynucleotide (P) is 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, or 120:1. In certain embodiments, the polymer-to-DNA (w/w) ratio is 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, or 200:1. In certain embodiments the ratio of N to P is between 30:1 and 60:1. The solution containing polymer and nucleic acid may be incubated for a period of time, e.g., for 1 minute up to several hours, e.g., for approximately 30 min to 1 hour. The solution may be incubated at room temperature, but higher or lower temperatures could be used. Formation of a polymer/nucleic acid complex may be assessed in a number of ways. For example, a portion of the sample may be run on an agarose gel in the presence of ethidium bromide and nucleic acid can be visualized under ultraviolet illumination. Complex formation results in retardation of the nucleic acid relative to the speed with which non-complexed nucleic acid migrates through the gel.

A number of cationic lipids facilitate uptake of nucleic acids by cells and can be used as delivery vehicles for the polynucleotides of the invention. Suitable cationic lipids include, but are not limited to, N-[1-(2,3-dioleoyloxy)propyl]-N,N,N-trimethylammonium chloride (DOTMA), 1,2-dioleoyl-3-trimethylammonium propane (DOTAP), dimethyldioctadecylammonium bromide (DDAB), cholesterol (CHOL), and 1,2-dioleoylphosphatidylethanolamine (DOPE). Mixtures of the foregoing can be used, e g., a mixture of DOTMA, DOTAP, or DDAB with CHOL or DOPE. Various ratios (e.g., equimolar amounts) can be used. The cationic lipids can be in the form of liposomes. Methods for preparation and use of cationic lipids and liposomes, including targeted liposomes, for delivery of nucleic acids in vitro and in vivo are well known in the art (95-102).

In certain embodiments of the invention the delivery vehicle is cell type selective, i.e., a composition comprising the delivery vehicle and a therapeutic agent does not display activity in one or more cell or tissue types, or displays significantly lower activity in one or more cell or tissue types, relative to its activity in other cell or tissue types, even though the therapeutic agent itself is not cell type specific. For example, certain delivery vehicles do not effectively mediate activity of a gene therapy vector in a particular cell type even though the gene therapy vector comprises a regulatory element that is otherwise known to be active in that cell type. The delivery vehicle may either prevent uptake of the therapeutic agent into the cells or may block activity of the agent if taken up. For example, a delivery vehicle/agent complex may be endocytosed but then trapped within endosomes in the particular cell type but not in other cells, resulting in cell type and tissue selectivity. The level of activity in a nontarget cell may be less than 70%, 60%, 50%, 40%, 30%, 20%, 10%, 5%, or 1% of the activity in a target cell.

In certain embodiments of the invention a delivery vehicle does not result in expression of a nucleic acid in striated muscle cells when the cells are contacted with a composition comprising the delivery vehicle and the nucleic acid but does result in expression of the nucleic acid in one or more other cell types when such cell types (e.g., cell types present within a hypertrophic tissue) are contacted with the composition. As described in Example 4, the inventors have shown that local injection of a polymer:DNA complex can successfully lead to expression of a protein encoded by the DNA in a variety of different normal cell types, but does not result in expression in striated muscle.

The ability to selectively avoid expression in striated muscle may be particularly valuable for treatment of hypertrophic tissues (other than skeletal muscle) that are in close proximity to skeletal muscle. For example, as mentioned above, the apex of the prostate gland is continuous with the striated muscle of the urethral sphincter, and normal prostatic glands can be found extending into the striated muscle. It would be desirable to avoid harming the muscle of the urethral sphincter. The thyroid gland is located close to various neck muscles. In the case of Graves' ophthalmopathy, the target tissue is located close to orbital muscles responsible for movement of the eyeball.

It may be desirable to utilize a composition of the invention in tissue culture, e.g., to selectively remove cells of a particular type from a population of cells of different types, to compare the efficacy of different therapeutic agents, to measure uptake or expression of an agent such as a nucleic acid in the presence or absence of a delivery vehicle, etc. Nucleic acids can be introduced into cells using methods known in the art such as transfection, electroporation, DEAE transfection, lipofection, microinjection, viral packaging, etc. Nucleic acids can also be introduced into cells as nucleic acid/polymer complexes, as described above.

In certain embodiments of the invention a viral delivery vehicle is used to introduce a cell type specific therapeutic agent such as the cell type specific nucleic acids described above into cells. The polynucleotide may be inserted into a naturally occurring or modified viral genome or a portion thereof or may be present within the virus or viral capsid as a separate nucleic acid molecule. A number of viral vectors have been used for gene therapy for a number of different diseases, and methods for their modification and use are well known in the art (83-91). These vectors include, but are not limited to, retroviral and lentiviral vectors (84-86), herpes simplex virus vectors (87, 88), adenoviral vectors (89), adeno-associated viral vectors (90), and vaccinia virus vectors (91).

C. Targeted Delivery Vehicles

In certain embodiments of the invention a delivery vehicle is targeted to a particular cell type, e.g., a cell type whose proliferation is at least in part responsible for causing inappropriate or excessive tissue growth. A number of methods for achieving targeted delivery are known in the art. For example, an antibody (preferably a monoclonal antibody) or ligand that specifically binds to a cell type specific marker may be covalently or noncovalently attached to or incorporated into a delivery vehicle such as a polymer, liposome, etc. using methods known in the art (60). See, also, Hermanson, G T., Bioconjugate Techniques, Academic Press, San Diego, 1996, which discusses a wide variety of methods for conjugating biomolecules to one another or to other molecules. The cell type specific marker may be a transmembrane or cell surface protein such as a receptor, ion channel, etc.

A variety of targeting agents that direct compositions to particular cells are known in the art (68). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialoglycoproteins, gp120 envelope protein of the human immunodeficiency virus (HIV), carbohydrates, receptor ligands, sialic acid, etc. If the targeting agent is included throughout the particle, the targeting agent may be included in the mixture that is used to form the particles. If the targeting agent is only on the surface, the targeting agent may be associated with (i.e., by covalent, hydrophobic, hydrogen boding, van der Waals, or other interactions) the formed particles using standard chemical techniques.

For treatment of BPH it may be desirable to target a therapeutic composition to a marker, e.g., a molecule such as a protein, that is present on or at the surface of prostate gland cells, e.g., luminal cells and/or basal cells of the prostate gland. By “on or at the surface of a cell” is meant that the molecule or a portion thereof is accessible to a targeting agent present in the extracellular environment so that it can be recognized and bound by the targeting agent. The molecule may be entirely extracellular, e.g., attached to the cell membrane, may be a transmembrane protein, etc. The molecule may be inserted into the cell membrane and may be partly or entirely within the membrane. In the latter case the targeting agent must partially penetrate the membrane to gain access. Suitable cell type specific markers present on or at the surface of prostate gland cells include PSMA, PSCA, etc. Additional markers include cluster designation (CD) antigens on the surface of prostate cells (95). Viruses can also be used to deliver a therapeutic agent specifically to one or more cell types. Certain viruses display tissue tropism in that they will only infect cells of particular types, e.g., cells that express a receptor for the virus on their cell surface. Such viruses can be used to deliver a therapeutic nucleic acid or vector encoding a therapeutic nucleic acid or polypeptide to a cell expressing the receptor for the virus. Viruses or viral capsids can also be modified with antibodies or ligands, engineered to express an antibody chain or ligand on the surface of the viral capsid, pseudotyped, or modified in other ways to target them to specific target cell types (86).

D. Local Delivery

In certain embodiments of the invention the composition is delivered locally. A variety of different types of compositions can be delivered locally. In certain embodiments of the invention the composition comprises a liquid. A liquid composition can comprise a therapeutic agent dissolved, suspended, or dispersed therein. The therapeutic agent may be a nucleic acid, small molecule, protein, etc. Liquid compositions can comprise polymer/nucleic acid complexes. Liquid compositions can comprise solid nanoparticles or microparticles comprising a therapeutic agent. Local delivery of a liquid composition may be accomplished in a number of different ways that are known in the art. For example, a liquid composition may be injected directly into its intended target tissue or in the vicinity thereof. The composition may be delivered by needle and syringe, catheter, cannula, etc. The composition may be delivered during laparoscopy and/or using ultrasound guidance or other imaging guidance. A liquid composition can also be administered locally to its intended target tissue during surgery, in which case it can be delivered using a syringe or poured from a suitable vessel. Alternately, a material can be wetted with the composition and then used to apply the liquid composition to an area of tissue.

In certain embodiments of the invention the composition comprises a gel or forms a gel following local administration. Gels can be delivered locally, e.g., either by injection or by application to the target tissue, e.g., during surgery. Gels may be delivered as liquid compositions containing a material that forms a gel following introduction into the body. A solution containing the gel-forming material and a therapeutic agent may be prepared by combining the gel-forming material and therapeutic agent in solution using any suitable method, e.g., by adding the therapeutic agent to a solution containing the gel-forming material. In certain embodiments the composition forms a gel following introduction into the body, e.g., upon contact with a physiological fluid. The composition may also be capable of forming a gel upon contact with a fluid such as phosphate buffered saline, or other fluid containing appropriate ions. Thus the composition can be injected at an appropriate location, e.g., in the vicinity of a target tissue where it forms a gel. Alternately, a preshaped gel implant can be made, e.g., by introducing the solution into a mold or cavity of the desired shape and allowing gel formation to occur in the presence of a suitable concentration of a salt. The salt can be added either prior to or following the introduction of the solution into the mold or cavity. The mold or cavity can be, e.g., any structure that contains a hollow space or concave depression into which a solution can be introduced. In another embodiment, a film or membrane is formed from the gel-forming solution containing a therapeutic agent.

Release of the agent from the gel can occur by any mechanism, e.g., by diffusion of the agent out of the gel, as a result of breakdown of the gel, or both. In certain embodiments of the invention the gel-forming material also comprises at least some solid material in addition to soluble material, which may modulate the rate of release of the therapeutic agent.

A variety of different gel-forming materials can be used in the present invention. Preferably the gel is a hydrogel, by which is meant a gel that contains a substantial amount of water. Preferably the material and the gel that it forms are biocompatible. Preferably the material and the gel that it forms are biodegradable.

Gel-forming materials of use in the invention include, but are not limited to, hyaluronic acid and modified forms thereof, polysaccharides such as alginate and modified forms thereof, collagen, self-assembling peptides, etc. See, e.g., U.S. Pat. No. 6,129,761 for further description of alginate and modified forms thereof, hyaluronic acid and modified forms thereof, and additional examples of soluble gel-forming materials that are of use in various embodiments of the present invention. As described therein, other polymeric hydrogel precursors include polyethylene oxide-polypropylene glycol block copolymers such as Pluronics™ or Tetronics™ which are crosslinked by hydrogen bonding and/or by a temperature change, as described in Steinleitner et al., Obstetrics & Gynecology, 77:48-52 (1991); and Steinleitner et al., Fertility and Sterility, 57:305-308 (1992). Other materials which may be utilized include proteins such as fibrin or gelatin. Polymer mixtures also may be utilized. For example, a mixture of polyethylene oxide and polyacrylic acid which gels by hydrogen bonding upon mixing may be utilized. Specific examples of hydrogels that are usable for delivery of therapeutic agents, including nucleic acids, have been described (93, 94; see also U.S. Pat. No. 6,129,761).

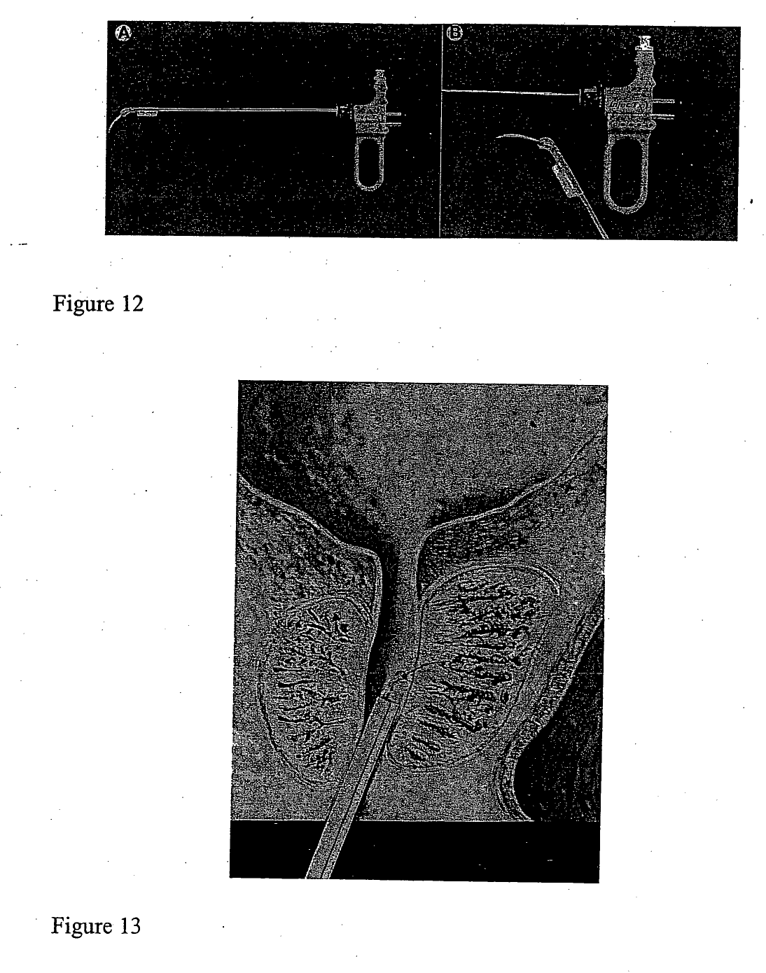

For treatment of BPH, a therapeutic composition in substantially liquid form can be injected into the prostate gland using a number of different routes known in the art including transperineal, transrectal, or transurethral, (57-59). For transurethral injection, a curved needle may be used. Such a device, marketed under the name ProstaJect™ (American Medical Systems, Minnetonka, Minn.) can be used (57). Another suitable device is the InjectTx endoscopic device (Injectx Inc., San Jose, Calif.) (59). The device is shown in FIGS. 12A and 12B. FIG. 13 shows use of the device to inject a composition of the invention into hypertrophic prostate gland tissue. Conventional methods for injection may be used for treatment of excessive or undesired thyroid, adipose, breast, gingival tissues, etc.

In certain embodiments of the invention the composition may be a drug delivery device comprising a solid material such as polymeric matrix impregnated with, or encapsulating, a therapeutic agent. The device may be shaped as a rod, disk, wafer, tube, sheet, or the like. The device is implanted into the body at the location of the target tissue or in the vicinity thereof, e.g., using conventional surgical techniques. For example, the device may be implanted into the prostate gland, thyroid gland, adipose tissue, breast, etc. Solid microparticles or nanoparticles, preferably biodegradable, comprising a therapeutic agent can also be implanted. The microparticles or nanoparticles may be contained within a second polymeric matrix or other drug delivery device. The therapeutic agent is typically released from the polymer over a period of time, e.g. by diffusion out of the matrix or release into the extracellular environment as the matrix degrades or erodes, as mentioned above.

V. Therapeutic Agents

A wide variety of therapeutic agents may be incorporated into the composition. In certain embodiments of the invention the therapeutic agent has a cytotoxic and/or cytostatic effect on cells, i.e., it kills cells and/or inhibits their survival (cytotoxic) and/or inhibits their proliferation (cytostatic). Cytotoxic and cytostatic agents are often used for the treatment of cancer and other diseases of a potentially life-threatening and/or severely debilitating nature. One aspect of the invention is the recognition that certain cytotoxic or cytostatic agents can be used to treat excessive or inappropriate noncancerous tissue growth without causing unacceptable toxicity or side effects. Another aspect of the invention is the provision of suitable compositions comprising a cytotoxic or cytostatic therapeutic agent or a polynucleotide that encodes a cytotoxic or cytostatic polypeptide.

In certain embodiments of the invention the composition comprises a nucleic acid. A therapeutic nucleic acid may act directly or indirectly on one or more cellular molecules or may comprise a template for transcription of a polypeptide that acts directly or indirectly on one or more cellular molecules.

In certain embodiments of the invention the composition comprises a vector, e.g., a gene therapy vector. Gene therapy encompasses delivery of nucleic acids comprising templates for synthesis of a therapeutic molecule, e.g., a therapeutic polynucleotide or polypeptide, to a cell of interest. The nucleic acid (or a nucleic acid derived from the nucleic acid as, for example, by reverse transcription) may be incorporated into the genome of the cell or remain permanently in the cell as an episome. However, gene therapy also encompasses delivery of nucleic acids that do not integrate or remain permanently in the cell to which they are delivered. Such approaches permit temporary or transient synthesis of a molecule of interest. For example, in the case of a gene that encodes a cytotoxic agent intended to kill the cell within which it is expressed, there is typically no need for continued expression once a sufficient amount of the agent has been synthesized to kill the cell.

In some embodiments of the invention a cytotoxic or cytostatic polypeptide is used to kill cells in hypertrophic tissues. For example, in certain embodiments diphtheria toxin A chain is used. Naturally occurring diphtheria toxin (DT) is produced by Corynebacterium diphtheriae as a secreted precursor polypeptide that is then enzymatically cleaved into two fragments, the A and B chains. The B chain binds to the surface of most eukaryotic cells and then delivers the A chain (DT-A) into the cytoplasm, where it inhibits proteins synthesis (61, 62, 109, 110). It is extremely toxic; a single molecule is sufficient to kill a cell (62).

In certain embodiments of the invention a polynucleotide that encodes the cytotoxic or cytostatic polypeptide is delivered to target cells and the polypeptide is synthesized within the cells. The DT gene has been cloned, sequenced, and adapted for expression in mammalian cells. A DT gene, DT-A, engineered for use in mammalian cells, encodes the DT-A subunit but not the DT-B subunit (63). The DT-A subunit is retained within the cytoplasm of the cell. In the absence of the B subunit, DT-A released from dead cells is not able to enter neighboring cells, thus ensuring that the toxin only kills cells in which it is expressed or to which it is targeted.

As described in Examples 2, 3, and 7, the inventors have expressed diphtheria toxin A chain (DT-A) in prostate cancer cells, prostate cancers, and normal prostate gland tissue, and demonstrated a dramatic inhibitory effect on protein synthesis and cell growth. The average growth rate of prostate tumors injected with a polymer/DNA complex containing a DNA construct in which expression of Flp recombinase under control of a prostate-specific regulatory element (PSE-BC) activated expession of DT-A was suppressed 2-fold relative to controls (Example 3). In some cases growth was entirely inhibited while in other cases regression occurred. In initial experiments, injection of this construct into a lobe of a normal prostate gland essentially obliterated the injected lobe while the other lobe appeared normal (Example 7). Further experiments described in Examples 8 and 9 demonstrated that polymeric nanoparticle-mediated delivery of a polynucleotide encoding DT-A to the prostate causes apoptosis and results in gross abnormalities in prostate morphology.

A variety of other cytotoxic polypeptides and peptides are known and can be used in the present invention. A gene encoding any of these may be incorporated into a nucleic acid molecule in operable association, i.e., operably linked, with a suitable promoter as described above. In certain embodiments of the invention the cytotoxic or cytostatic polypeptide is a protein synthesis inhibitor. Polypeptides exhibiting cytotoxic or cytostatic activity include, but are not limited to, gibbon ape leukemia virus fusogenic membrane glycoprotein, Pseudomonas exotoxin A (PE), cholera toxin (CT), pertussis toxin (PT), ricin A chain, abrin A chain, modeccin A chain, botulinum toxin A, alpha-sarcin, dianthin proteins, momordica charantia inhibitor, curcin, crotin, sapaonaria officinalis inhibitor, gelonin, mitogellin, hirsutellin A, calcaelin, restrictocin, phenomycin, and enomycin.

Vectors and vehicles that provide nucleic acids comprising templates for synthesis of such molecules may be incorporated into a composition of the invention. In certain embodiments of the invention the nucleic acid includes a coding sequence for a therapeutic nucleic acid or polypeptide to be expressed in a cell type of interest and also includes appropriate regulatory elements, e.g., promoters, enhancers, operably linked to the coding sequence so as to ensure proper expression. In certain embodiments of the invention the regulatory elements are cell type specific, so that the gene will only be expressed in cells of a particular cell type or types, e.g., one or more cell types present in the prostate gland, adipose tissue, thyroid gland, etc. Suitable cell type specific regulatory elements are discussed above. Additional genetic elements, e.g., polyA signals, transcriptional terminators, etc., can also be included in the nucleic acid.

In certain embodiments of the invention the therapeutic agent is a nucleic acid that acts by reducing expression of a gene whose expression product is required for or contributes to cell division and/or survival (referred to as a target gene). Suitable target genes include cell cycle genes such as genes encoding cyclins or cyclin dependent kinases (CDKs), anti-apoptosis genes, etc. In general, any essential gene is a suitable target, though non-essential genes whose inhibition reduces cell survival or division can also be useful targets. While not wishing to be bound by any theory, inactivation of proapoptotic pathways may be involved in the development and/or progression of BPH. Therefore, methods of reducing expression of genes that protect prostate gland cells from apoptosis can be used for treatment of BPH. Such genes include, but are not limited to, genes that encode Akt kinase, elongation factor 4E-B1, NAIP, cIAP-1, cIAP-2, XIAP, and survivin (54 and references therein). Similar strategies may be employed for treatment of excessive or unwanted tissue growth in other tissues that is at least in part attributable to decreased apoptosis.

In addition, or instead of, reducing expression of genes that protect cells from apoptosis, expression of genes that contribute to apoptosis of prostate gland cells can be increased, e.g., by providing the gene to cells as described above for genes that encode cytostatic or cytotoxic products. Such genes include, but are not limited to, phosphatase and tensin homologue deleted on chromosome 10 (PTEN), BAD, and caspase-9 (54 and references therein). Similar strategies may be employed for treatment of excessive or unwanted tissue growth in other tissues.

Therapeutic nucleic acids that reduce expression of a target gene include, but are not limited to, siRNAs, shRNAs, antisense oligonucleotides, and ribozymes. Antisense nucleic acids are generally single-stranded nucleic acids (DNA, RNA, modified DNA, modified RNA, or peptide nucleic acids) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. Extensive discussion of ribozyme technology and its uses is found in Rossi, J. J., and Duarte, L. C., Intracellular Ribozyme Applications: Principles and Protocols, Horizon Scientific Press, 1999.

RNA interference (RNAi) is a mechanism of post-transcriptional gene silencing triggered by double-stranded RNA (dsRNA), which is distinct from antisense and ribozyme-based approaches. dsRNA molecules direct sequence-specific degradation of mRNA that contains regions complementary to one strand (the antisense strand) of the dsRNA in cells of various types after first undergoing processing by an RNase III-like enzyme (Bernstein et al., Nature 409:363, 2001) into smaller dsRNA molecules. These molecules comprise two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is mediated by naturally occurring or synthetic molecules of this structure, and other similar structures, which are referred to as short interfering RNAs (siRNAs). siRNAs typically comprise a double-stranded region approximately 19 nucleotides in length (but ranging between 12-29), optionally with 1-2 nucleotide 3′ overhangs on one or both strands, resulting in a total length typically between approximately 21 and 23 nucleotides.

RNAi can also be achieved using short hairpin RNAs (shRNA), which are single RNA molecules comprising at least two complementary portions capable of self-hybridizing to form a duplex structure sufficiently long to mediate RNAi (typically at least 19 base pairs in length), and a single-stranded loop, typically between approximately 1 and 10 nucleotides in length and more commonly between 4 and 8 nucleotides in length that connects the two nucleotides that form the last nucleotide pair at one end of the duplex structure. shRNAs are thought to be processed into siRNAs by the cellular RNAi machinery. Thus shRNAs are precursors of siRNAs and are similarly capable of inhibiting expression of a target transcript.

siRNAs and shRNAs have been shown to downregulate gene expression in mammalian subjects when delivered by various methods including intravenous or local injection. RNA interference using siRNA and/or shRNA is reviewed in, e.g., Tuschl, T., Nat. Biotechnol., 20: 446-448, May 2002; Dykxhoorn, D., et al., Nat Rev Mol Cell Biol. 4(6):457-67, 2003. See also Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002).

An siRNA, shRNA, antisense molecule, or ribozyme is considered “targeted” to an mRNA if the stability of the target transcript is reduced in the presence of the siRNA, shRNA, antisense molecule, or ribozyme as compared with its absence (or, for RNAs that act by inhibiting translation, translation of the target transcript is reduced in the presence of the RNA as compared with its absence). Typically in the case of siRNAs or shRNAs, the duplex portion of the siRNA or shRNA shows at least about 70%, preferably at least about 80%, preferably at least about 90%, more preferably at least about 91%, 92%, 93%, 94%, 95%, 96%, 97%, 98%, 99%, or 100% precise sequence complementarity with the target transcript for a stretch of at least 15, preferably at least 17, more preferably at least 18 or 19 to about 21-23 nucleotides. Typically at least part of the antisense portion of the siRNA or shRNA hybridizes to the target transcript under stringent conditions (selected taking into account the length of the part of the antisense portion that hybridizes with the target transcript).

Selection of appropriate siRNA and shRNA sequences can be performed according to guidelines well known in the art, e.g., taking factors such as desirable GC content into consideration. See, e.g., Ambion Technical Bulletion #506, available at the web site having URL www.ambion.com/techlib/tb/tb 506.html, visited in Octob er 2004 and on Oct. 20, 2005. Following these guidelines approximately half of the selected siRNAs effectively silence the corresponding gene, indicating that by selecting about 5 siRNAs it will almost always be possible to identify an effective sequence. A number of computer programs that aid in the selection of effective siRNA/shRNA sequences are known in the art, which yield even higher percentages of effective siRNAs. See, e.g., Cui, W., et al., “OptiRNai, a Web-based Program to Select siRNA Sequences”, Proceedings of the IEEE Computer Society Conference on Bioinformatics, p. 433, 2003. Pre-designed siRNAs targeting over 95% of the mouse or human genome are commercially available, e.g, from Ambion and/or Cenix Biosciences. See web site having URL www.ambion.com/techlib/tn/104/5.html.

Therapeutic nucleic acids can be delivered to cells within a subject as part of a composition, e.g., complexed with a poly(beta amino ester) or other delivery vehicle. Therapeutic nucleic acids can be expressed intracellularly, i.e., by introducing a vector that comprises a template for transcription of the nucleic acid into cells of the subject. siRNAs and shRNAs have been shown to effectively reduce gene expression when expressed intracellularly, e.g., by delivering vectors such as plasmids, viral vectors such as adenoviral, retroviral or lentiviral vectors, to cells. Such vectors, referred to herein as RNAi-inducing vectors, are vectors whose presence within a cell results in transcription of one or more RNAs that self-hybridize or hybridize to each other to form an shRNA or siRNA. In general, the vector comprises a nucleic acid operably linked to regulatory elements (promoters, enhancers, etc.) so that one or more RNA molecules that hybridize or self-hybridize to form an siRNA or shRNA are transcribed when the vector is present within a cell. The regulatory element(s) can be cell type specific. The vector provides a template for intracellular synthesis of the RNA or RNAs or precursors thereof. The vector will thus contain a sequence or sequences whose transcription results in synthesis of two complementary RNA strands having the properties of siRNA strands described above, or a sequence whose transcription results in synthesis of a single RNA molecule containing two complementary portions separated by an intervening portion that forms a loop when the two complementary portions hybridize to one another. Vectors that provide templates for transcription of a therapeutic nucleic acid can be delivered to subjects as described elsewhere herein.

In other embodiments of the invention, the therapeutic agent is a cytostatic or cytotoxic compound. Cytotoxic or cytostatic polypeptides such as those described above (e.g., diphtheria toxin A chain, botulimun toxin) can be incorporated directly into a composition, e.g., a composition comprising a cell type selective delivery vehicle such as a poly(beta amino ester) for delivery to a target tissue. Alternately, the therapeutic agent can be a cytostatic or cytotoxic agent that is used in cancer chemotherapy, of which many are known in the art including, but not limited to: alkylating agents; nitrosorureas; antimetabolites (structural analogs of compounds important in cellular metabolism), e.g., methotrexate, purine or pyrimidine analogs; plant alkaloids such as vinblastine, vincristine, podophyllotoxins, camptothecins, and taxanes; antibiotics (compounds originally isolated from microorganisms) such as anthracyclines, mitomycin, bleomycin, asparaginase; hormonal agents such as estrogen and/or androgen inhibitors (e.g., tamoxifen) and aromatase inhibitors; hydroxyurea; etc. The compositions may also be given in conjunction with agents of more recently developed classes of chemotherapeutic agents such as kinase inhibitors, famesyltransferase inhibitors, mTOR pathway inhibitors such as rapamycin or rapamycin analogs, other oncogene or cell cycle inhibitors.

Tissue hypertrophy may occur at least in part due to deposition or collection of noncellular material such as lipid, extracellular matrix components such as collagen and proteoglycans, etc. In certain embodiments of the invention a cell type specific therapeutic agent reduces cell division and/or kills a cell that produces such material. Certain therapeutic compositions may contain a substance that degrades, dissolves, or otherwise facilitates removal of such materials. For example, certain therapeutic compositions comprise a protease such as collagenase, chondroitinase, hyaluronidase, etc., a lipase, and/or an agent such as plasmin or tissue plasminogen activator that contributes to dissolving blood clots, in addition to or instead of one or more other therapeutic agents.

VI. Transgenic Animal Model

The invention provides a transgenic nonhuman animal whose genome contains (i) a transgene comprising a first regulatory element that directs expression in luminal cells but not basal cells, wherein the first regulatory element is operably linked to a nucleic acid sequence that encodes a first detectable marker and (ii) a transgene comprising a second regulatory element that directs expression in basal cells but not luminal cells, wherein the second regulatory element is operably linked to a nucleic acid sequence that encodes a second detectable marker, wherein the first and second detectable markers are distinguishable from each other. The first regulatory element can be a luminal cell specific regulatory element. The second regulatory element can be a basal cell specific regulatory element. The luminal cell specific regulatory element may be specific for luminal cells of one or more different glands. The basal cell specific regulatory element may, but need not be, specific for basal cells of one or more different glands. The luminal and basal cells may be cells of a particular gland, e.g., an endocrine gland or exocrine gland. For example, the gland may be the prostate gland, thyroid gland, etc. The

A transgene is exogenous DNA or a rearrangement, e.g., a deletion of endogenous chromosomal DNA, which preferably is integrated into or occurs in the genome of the cells of a transgenic animal. Preferably the transgene comprises a promoter operably linked to a nucleic acid such that expression of the nucleic acid occurs in the cell. Certain preferred transgenic animals are non-human mammals, e.g., rodents such as rats or mice. Other examples of transgenic animals include sheep, dogs, cows, and goats. Methods for making transgenic animals such as these are known in the art.

In general, a detectable marker is a marker whose presence within a cell can be detected through means other than subjecting the cell to a selective condition or directly measuring the level of the detectable marker itself. Thus in general, the expression of a detectable marker within a cell results in the production of a signal that can be detected and/or measured. The process of detection or measurement may involve the use of additional reagents and may involve processing of the cell. For example, where the detectable marker is an enzyme, detection or measurement of the marker will typically involve providing a substrate for the enzyme. Preferably the signal is a readily detectable signal such as light, fluorescence, luminescence, bioluminescence, chemiluminescence, enzymatic reaction products, or color. Thus preferred detectable markers for use in the present invention include fluorescent proteins such as green fluorescent protein (GFP) and variants thereof. A number of enhanced versions of GFP (eGFP) have been derived by making alterations such as conservative substitutions in the GFP coding sequence. Certain of these enhanced versions of GFP display increased fluorescence intensity or expression relative to wild type GFP and may be preferred. Other detectable markers that produce a fluorescent signal include red, blue, yellow, cyan, and sapphire fluorescent proteins, reef coral fluorescent protein, etc. A wide variety of such detectable markers is available commercially, e.g., from BD Biosciences (Clontech). Additional detectable markers preferred in certain embodiments of the invention include luciferase derived from the firefly (Photinus pyralis) or the sea pansy (Renilla reniformis). In addition, a detectable signal can be a detectable alteration in a biological pathway or response to an agent, e.g., a chemical agent.

As described in Example 10, a double transgenic mouse model in which cyan fluorescent protein (CFP) and GFP reporter genes are discriminately expressed in basal and luminal cells, respectively, in the prostatic epithelium was used to show that a chimeric promoter/enhancer of the human PSA gene effectively targets the expression of DT-A to luminal cells in the prostate, resulting in their death. Nonhuman transgenic animals of the invention, such as this mouse model, are useful for preclinical testing of other therapies targeting the prostate, as well as for other studies aimed at understanding basic prostate biology.

VIII. Therapeutic Applications

In general, the methods and compositions of the invention are useful for the treatment of any disease or condition associated with tissue hypertrophy and/or hyperplasia and other forms of unwanted tissue growth such as obesity. In particular, the methods and compositions are useful for the treatment of BPH as described above. Compositions and methods of the invention may be tested in a variety of animal models. As described above, one aspect of the invention is a transgenic nonhuman animal model in which the effects of a composition on prostate cells can be tested, and differential effects on basal and luminal cells of the prostate can be evaluated. Alternative or additional animal models can be used. For example, both canine and primate (chimpanzee) animal models of BPH are known.

Thyroid conditions such as multinodular goiter and Graves' disease, both of which are associated with an increase in thyroid gland tissue, can also be treated. Hyperthyroidism is a syndrome in which tissue is exposed to excessive amounts of circulating thyroid hormone (55 and references therein). There are a number of different causes including Graves' disease (an autoimmune condition resulting from stimulation of the thyroid by antibodies directed against the thyrotropin (TSH) receptor), toxic multinodular goiter, and solitary hyperfunctioning thyroid nodules. Hyperthyroidism is conventionally treated using surgery, radioactive iodine, and/or anti-thyroid drugs. The invention offers an alternative approach. In accordance with the invention hyperthyroidism is treated by local delivery of a composition comprising a therapeutic agent that inhibits growth of thyroid gland cells or kills thyroid gland cells. The composition may comprise a cell type specific therapeutic agent, e.g., a vector that directs expression of a therapeutic nucleic acid or polypeptide in a thyroid cell specific manner using, for example, any of the thyroid cell specific regulatory elements discussed above. The composition may comprise a tissue-selective delivery vehicle such as a beta (poly amino) ester. The composition may be injected into the thyroid gland. Where one or more discrete nodule(s) can be identified, the composition may be injected directly into the nodule(s).

Graves' disease causes a diffuse enlargement of the thyroid gland and is often associated with Graves' ophthalmopathy, a condition characterized by an expansion of extraocular muscle tissue, orbital adipose tissue, or both (55, 56). Fat and muscle expansion causes compression of the orbital contents. Graves' ophthalmopathy can be treated by injecting a composition of the invention comprising either a nucleic acid based (e.g., therapeutic nucleic acid, vector that directs expression of a therapeutic nucleotide or polypeptide) or conventional cytotostatic or cytotoxic agent into extraocular muscle and/or adipose tissue. A therapeutic nucleic acid or polypeptide can be expressed in orbital and/or adipose tissue in a subject with Graves' ophthalmopathy using a regulatory element derived from the TSH-R gene, which is expressed in orbital/connective tissue specimens and cultures in subjects with Graves' ophthalmopathy (56).

The compositions and methods of the invention can be used to reduce drug-induced tissue hypertrophy. A number of medications are known to cause gingival hypertrophy as a side effect. Among these are calcium channel blockers (Samarasinghe Y P, Calcium channel blocker induced gum hypertrophy: no class distinction, Heart, 90(1):16, 2004), cyclosporine (Meraw S J and Sheridan P J. Medically induced gingival hyperplasia, Mayo Clin Proc., 73(12):1196-9, 1998), and anticonvulsants, particularly phenytoin (Brunet L, et al., Prevalence and risk of gingival enlargement in patients treated with anticonvulsant drugs, Eur J Clin Invest, 31(9):781-8 (2001)). In accordance with certain embodiments of the invention a composition comprising a therapeutic agent that inhibits growth of gingival cells or kills such cells is locally delivered (e.g., by injection) into the hypertrophic gum tissue. The composition may comprise a tissue-selective delivery vehicle such as a beta (poly amino) ester.

Obesity, or any medically and/or cosmetically undesirable accumulations of adipose tissue can also be treated. A variety of genes that are selectively or specifically expressed in adipose tissue have been identified, as mentioned above. In accordance with the invention obesity or an undesired accumulation of adipose tissue is treated by local delivery of a composition comprising a therapeutic agent that inhibits growth of adipose cells or kills such cells. The composition may comprise a cell type specific therapeutic agent, e.g., a vector that directs expression of a therapeutic nucleic acid or polypeptide in an adipose cell specific manner using, for example, regulatory elements derived from any of the adipose cell specific genes discussed above. The composition may comprise a tissue-selective delivery vehicle such as a beta (poly amino) ester. Preferably a composition comprising a tissue-selective or specific therapeutic agent, and optionally comprising a tissue-selective delivery vehicle is delivered locally, e.g., by injection or implantation, at a site of adipose tissue whose reduction in size is desired. Breast tissue may also be reduced in an analogous manner using the compositions and methods of the invention.

Benign tumors such as leiomyomas, accumulations of fibrous tissue, scars, etc., can also be treated. Cysts, e.g., dermoid cysts, epidermal cysts, etc., that have a component of cell proliferation can also be treated by local administration of a composition of the invention comprising a cytotoxic or cytostatic therapeutic agent and, optionally, a tissue-selective delivery vehicle.

IX. Pharmaceutical Compositions and Additional Delivery Methods

Suitable preparations, e.g., substantially pure preparations of therapeutic agents that inhibit cell survival or proliferation, optionally together with a delivery vehicle such as a poly (beta-amino ester) may be combined with pharmaceutically acceptable carriers, diluents, solvents, etc., to produce a pharmaceutical composition. Any of the compositions described herein may be formulated as a pharmaceutical composition suitable for administration to patients. In certain embodiments of the invention the pharmaceutical composition detectably reduces tissue volume or inhibits continued growth of the tissue. In other words, administration of the composition measurably reduces tissue volume relative to the volume that would exist in the absence of the composition. It is to be understood that the pharmaceutical compositions of the invention, when administered to a subject, are preferably administered for a time and in an amount sufficient to treat or prevent the disease or condition for whose treatment or prevention they are administered.

Further provided are pharmaceutically acceptable compositions comprising a pharmaceutically acceptable derivative (e.g., a prodrug) of any of the therapeutic agents of the invention, by which is meant any non-toxic salt, ester, salt of an ester or other derivative of a compound that, upon administration to a recipient, is capable of providing, either directly or indirectly, the effect of a therapeutic agent of the invention.

In preferred embodiments of the invention therapeutic compositions are delivered locally to hypertrophic tissues, e.g., as described above. However, in other embodiments compositions may be formulated for delivery by any available route including, but not limited to parenteral, oral, by inhalation to the lungs, nasal, bronchial, ophthalmic, transdermal (topical), transmucosal, rectal, and vaginal routes. The term “parenteral” as used herein includes subcutaneous, intravenous, intramuscular, intra-articular, intra-synovial, intrasternal, intrathecal, intrahepatic, intralesional and intracranial injection or infusion techniques.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, antibacterial and antifungal agents, isotonic and absorption delaying agents, and the like, compatible with pharmaceutical administration may be included. Supplementary active compounds, e.g., compounds independently active against the disease or clinical condition to be treated, or compounds that enhance activity of an inventive compound, can also be incorporated into the compositions.

Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The composition should be sterile, if possible, and should be fluid to the extent that easy syringability exists if it is to be delivered by means that use a syringe.

Preferred pharmaceutical formulations are stable under the conditions of manufacture and storage and may be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of injectable compositions can be brought about by including in the composition an agent which delays absorption, for example, aluminum monostearate and gelatin. Prolonged absorption of oral compositions can be achieved by various means including encapsulation.

Sterile injectable solutions can be prepared by incorporating the active agent in the required amount in an appropriate solvent with one or a combination of ingredients enumerated above, as required, followed by filtered sterilization. Preferably solutions for injection are free of endotoxin. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active agent can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions are preferably delivered in the form of an aerosol spray from a pressured container or dispenser which contains a suitable propellant, e.g., a gas such as carbon dioxide, or a nebulizer. Liquid or dry aerosol (e.g., dry powders, large porous particles, etc.) can be used. The present invention also contemplates delivery of compositions using a nasal spray.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In addition to the delivery vehicles described above, in certain embodiments of the invention, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polyethers, and polylactic acid. Methods for preparation of such formulations will be apparent to those skilled in the art and are discussed above. Certain of these materials can also be obtained commercially from Alza Corporation and Nova Pharmaceuticals, Inc. Liposomal suspensions can also be used as pharmaceutically acceptable carriers (see above). These can be prepared according to methods known to those skilled in the art, for example, as described in U.S. Pat. No. 4,522,811 and other references listed herein.

It is typically advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 100 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition can include a single treatment or, in many cases, can include a series of treatments.

Exemplary doses include milligram or microgram amounts of the inventive compounds per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) In some embodiments of the invention doses much smaller than these may be used. It is furthermore understood that appropriate doses depend upon the potency of the agent, and may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, the amount of tissue to be reduced, and the amount of reduction desired.

The present invention includes the use of inventive compositions for treatment of nonhuman animals including, but not limited to, companion animals such as dogs and cats, agriculturally important animals such as ruminants (e.g., cows), sheep, horses, etc. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

The invention further provides pharmaceutical compositions comprising two or more therapeutic agents of the invention, e.g., two or more nucleic acid constructs such as those described above. The invention further provides a pharmaceutical composition comprising a therapeutic agent of the invention and a second agent, e.g., a hormone, anti-thyroid drug, etc.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. Furthermore, where the claims recite a composition, it is to be understood that methods of administering the composition according to any of the methods disclosed herein, and methods of using the composition for any of the purposes disclosed herein are included, and methods of making the composition according to any of the methods of making disclosed herein are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein.

The inclusion of a “providing” step in certain methods of the invention is intended to indicate that the composition or device is administered to treat a disease or condition characterized by inappropriate or excessive noncancerous tissue growth, e.g., BPH. Thus the subject will have or be at risk of a disease or condition characterized by inappropriate or excessive noncancerous tissue growth, and the composition or device is administered to treat the disorder, typically upon the sound recommendation of a medical or surgical practitioner, e.g., a urologist in the case of BPH, who may or may not be the same individual who administers the composition or device. Typically the subject will not have been diagnosed with cancer in the same tissue as that which exhibits inappropriate or excessive noncancerous tissue growth or, if the subject has been so diagnosed, the subject also exhibits inappropriate or excessive noncancerous growth in the same tissue. For example, typically the subject will not have been diagnosed with prostate cancer or, if the subject has been diagnosed with prostate cancer, the subject also has concomitant inappropriate or excessive prostate tissue. The invention includes embodiments in which a step of providing is not explicitly included and embodiments in which a step of providing is included. The invention also includes embodiments in which a step of identifying the subject as being at risk of or suffering from a disease or condition characterized by inappropriate or excessive noncancerous tissue growth, e.g., BPH, is included.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g,. any polypeptide or polynucleotide), any method of administration, any disorder or condition or characteristic(s) thereof, or any subject characteristic(s) can be excluded from any one or more claims, for any reason, whether or not related to the existence of prior art.

### EXAMPLES

### Example 1

### Synthesis and Screening of a Library of Poly(β-Amino Esters)

Materials and Methods

Polymer Synthesis. Monomers were purchased from Aldrich (Milwaukee, Wis.), TCI (Portland, Oreg.), Pfaltz & Bauer (Waterbury, Conn.), Matrix Scientific (Columbia, S.C.), Scientific Polymer (Ontario, N.Y.), and Dajac monomer-polymer (Feasterville, Pa.). Six to twelve versions of each polymer were generated by varying the amine/diacrylate stoichiometric ratio. To synthesize each polymer, 500 mg of amino monomer was weighed into an 8 ml sample vial with Teflon-lined screw cap. Next, the appropriate amount of diacrylate was added to the vial to yield a stoichiometric ratio ranging from 0.6 to 1.4. A small Teflon-coated stir bar was then put in each vial. Polymers were then synthesized on a multi-position magnetic stir-plate residing in an oven at 1) 95° C. and solvent free, or 2) 60° C. with 2 ml DMSO added. High temperature synthesis was performed for approximately 12 hours, and low temperature synthesis was performed for 2 days. After completion of reaction, all vials were removed from the oven and stored at 4° C. Luciferase Transfection Assays were performed as described in (12).

Measurement of Cytotoxicity. COS-7 cells (ATCC, Manassas, Va.) were seeded (14,000 cells/well) into clear plates. After 24 hours, increasing amounts of polymer, from 10-800 μg/ml, in Opti-MEM® medium were added to the cells. Cells were incubated with the polymer for 1 hour, and then media was replaced and metabolic activity was measured using the MTT Cell Proliferation Assay kit (ATCC) after 1 day. 10 μl of MTT Reagent was added to each well. After 2 hr incubation at 37° C., 100 μl of Detergent Reagent was added to each well. The plate was then left in the dark at room temperature for 4 hr. Optical absorbance was measured at 570 nm using a SpectraMax 190 microplate reader (Molecular Devices, Sunnyvale, Calif.) and converted to % viability relative to control (untreated) cells.

Cells. PC3 cells (ATCC, Manassas, Va.) were cultured in Dulbecco's Modifed Eagle's Medium supplemented with 10% fetal bovine serum (FBS). LNCaP cells (UroCor, Inc., Oklahoma City, Okla.) were cultured in RPMI 1640 medium supplemented with 10% FBS. PC3 and LNCaP cells were maintained at 37° C. in 5% CO₂, balance air.

Results

Our previous work with poly(β-amino esters) identified several monomers as frequently present in effective gene delivery polymers (11). In general, the most effective polymers were composed of primary amino monomer containing an alcohol, imidazole, or a secondary di-amine. Acrylate monomers in effective polymers were almost always hydrophobic (FIG. 1B). However, since both molecular weight and end-group termination can have order of magnitude effects on the transfection potential of poly(β-amino esters) (12), we sought to further optimize polymer transfection potential.

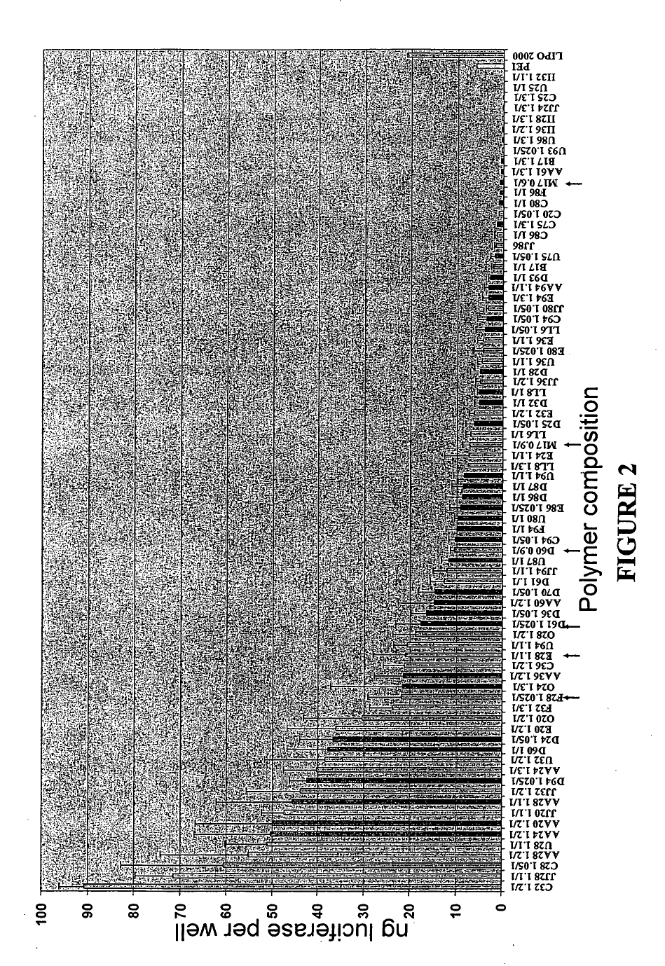

Differences in the stoichiometric ratio of amine monomer to acrylate monomer ranging from 0.6 to 1.4 substantially affect the molecular weight of poly(β-amino esters) and their DNA transfection efficiency (12). In particular, it was observed that polymers formed with an excess of amine monomer tend to be more effective. We therefore synthesized some polymers at amine:acrylate ratios of 0.6 and 1.4, and at 10 different ratios between these values (0.6, 0.8, 0.9, 0.95, 0.975, 1.0, 1.025, 1.05, 1.1, 1.2, 1.3, and 1.4). To allow for greater control of monomer stoichiometry, and therefore better control over polymer molecular weight and chain end-groups, polymer synthesis was scaled up to gram amounts. All polymers were synthesized by adding acrylate monomer, resulting in the appropriate stoichiometric ratio, to 500 mg of amine monomer. A total of 70 monomer combinations were used, with 6 to 12 monomer ratios for each combination (FIG. 2). Polymerizations were first performed at 95° C. in the absence of solvent to maximize monomer concentration, or at 60° C. in the presence of 2 ml DMSO to reduce potential hardening.

We performed in vitro transfection assays using all polymers at 6 different polymer:DNA ratios to determine the transfection efficiency of each polymer. COS-7 cells were transfected with plasmid DNA encoding the firefly luciferase reporter gene (PCMV-Luc). To facilitate performance of the over 12,000 transfections (data obtained in quadruplicate), experiments were done in 96-well plate format. Luciferase expression levels were determined using a commercially available luciferase assay kit and a 96-well luminescence plate reader.

The transfection efficiency of polymers synthesized at the optimal monomer ratios and at the optimal polymer:DNA ratio, is shown in FIG. 2. Eighteen polymers transfected cells with higher efficiency than did Lipofectamine (21 ng luciferase/well) and 43 performed better than PEI (polymer:DNA=1:1 w/w, 6 ng luciferase/well), under the same conditions. The polymers yielding the highest transfection efficiency were C32, JJ28, and C28 (91, 72, and 61 ng luciferase/well, respectively). These polymers, as well as other top-performing polymers, contain amines with alcohol groups and hydrophobic acrylates. It is important to note that the overall transfection levels and the monomer composition of the most effective polymers are both higher than and different from those identified in our preliminary, high-throughput screening library (11). These differences highlight the important influence of molecular weight, chain end-group identity, and polymer:DNA ratio on transfection efficiency.

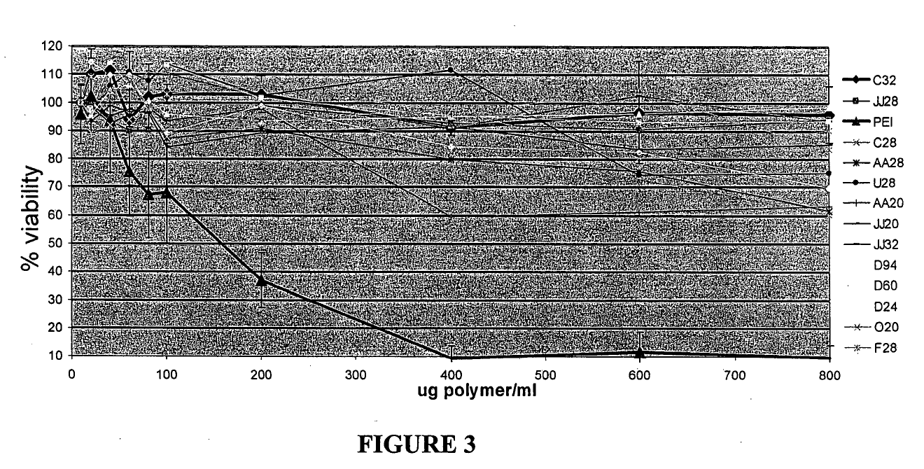

We tested 13 of the most effective gene delivery poly(β-amino esters) in vitro for cytotoxicity using the MTT assay (FIG. 3). COS-7 cells were incubated with varying amounts of polymer (10-800 μg/ml) in Opti-MEM® medium for 1 hour and then assayed 24 hours later. While treatment of cells with some polymers (e.g., AA20) resulted in some toxicity, especially at higher polymer concentrations, all of the poly(β-amino esters) were significantly less toxic than PEI. C32 (1.2:1 amine:acrylate ratio), the most efficient transfection polymer, was not toxic over the concentration range tested. Since C32 is both highly effective at gene delivery and demonstrates no toxicity in vitro, we chose to use this polymer for in vivo gene delivery studies. The studies described below utilize C32 at a 1.2:1 amine:acrylate ratio.

### Example 2

### C32-Delivered DNA Encoding Diphtheria Toxin (DT-A) Arrests Protein Synthesis in Prostate Cancer Cells In Vitro

Plasmid construction. pCAG/luc plasmid DNA, containing a firefly luciferase coding sequence regulated by a very strong, ubiquitously expressed promoter/enhancer was constructed as follows. A 1.7-kb fragment containing the luciferase coding sequence, released by digestion of pGL3-Basic vector (Promega, Madison, Wis.) with BglII and XbaI, was ligated to a 3.4-kb BamHI+XbaI fragment derived from pEGFP-1 (Clontech, Palo Alto, Calif.) to create pLucf. The plasmid pCX-EGFP (gift of J. Miyazaki, Kyoto U.), was digested with SalI and EcoRI to release a 1.7-kb fragment. This fragment, containing the CAG sequence, was ligated to a XhoI+EcoRI digest of pLucf to create pCAG/luc. CAG is composed of CMV enhancer sequence and the promoter sequence of the chicken β-actin gene.

The plasmid RSV/FRT2PSA.FLP/EGFP was constructed as follows. A 2-kb fragment containing Flp recombinase sequence, released by digestion of pOG-FLPe6 (gift of A. Francis Stewart, EMBL, Heidelberg) with XbaI and SalI, was ligated to XbaI+XhoI-digested pMECA (15) to produce pMECA/FLP. This plasmid was then digested with XbaI and AgeI to release a 2-kb fragment that was ligated to NheI+NgoMI digested pMECA to create pMECA/FLP (2). A 2.5-kb fragment containing the PSE-BC promoter sequence, released from the plasmid pPSE-BC (gift of Lily Wu, UCLA) by digestion with XbaI and SalI), was ligated to XbaI+SalI-digested pMECA/FLPe(2) to create pMECA/PSA.FLP(dam−). A 4.5-kb fragment, released from pMECA/PSA.FLP by digestion with XbaI and AvrII, was ligated to NheI-digested pFRT2 (gift of Susan Dymecki, Harvard) to generate pFRT2/PSA.FLP. Finally, this plasmid was digested with AgeI and XmnI to release a 4.5-kb fragment that was ligated to AgeI-digested pRSV/EGFP to produce RSV/FRT2PSA.FLP/EGFP.

The plasmid pRSV/FRT2PSA.FLP/DT-A was constructed as follows. The plasmid p22EDT1 (gift of A. Francis Stewart, EMBL, Heidelberg) was digested with BglII and NotI, releasing a 1.3-kb fragment containing DT-A sequence that was ligated to a 5.0-kb fragment released from the plasmid pIND by BamHI+NotI digestion to create pIND/DT-A. pIND/DT-A was digested with KpnI and XbaI, releasing a 1.3-kb fragment which was ligated to a KpnI+XbaI digest of pMECA to create pMECA/DT-A. This plasmid was digested with AgeI and XbaI, releasing a 1.3-kb fragment which was ligated to a 3.8-kb fragment deriving from an AgeI+NheI digest of pRSV/EGFP. The resulting plasmid, pRSV/DT-A, was digested with AgeI, and then ligated to a 4.5-kb fragment released from pFRT2/PSA.FLP by digestion with AgeI and XmnI to create pRSV/FRT2PSA.FLP/DT-A.

The plasmid pRSV/EGFP, used in the above construction, was constructed as follows. pEGFP-1 (Clontech, Palo Alto, Calif.) was digested with BamHI and AflII. The resulting 1 kb fragment was ligated into the BamHI and AflII sites of pIND (Invitrogen, Carlsbad, Calif.) to create pIND/EGFP. pIND/EGFP was then digested with SpeI and NheI. The resulting 1 kb fragment was ligated into the NheI site of pDC312/RSV (5) to create pRSV/EGFP.

All restriction enzymes were purchased from Promega (Madison, Wis.). Salmon testes DNA (Sigma-Aldrich, St. Loius, Mo.) served as a negative control in xenograft experiments.

Polymer:DNA complex formation. To complex plasmid DNA to C32, the polymer was dissolved in dimethyl sulfoxide (100 mg/ml). DNA (50 μg) was suspended in 25 μl 25 mM sodium acetate buffer, pH 5.0, and mixed with C32 polymer (300 or 1500 μg), also diluted in 25 μl 25 mM sodium acetate buffer, pH 5.0. After incubation of the polymer/DNA mixture at room temperature for 5 minutes, 10 μl 30% glucose in PBS was added to the 50 μl polymer/DNA mixture.

Results

DT-A catalyzes the transfer of ADP-ribose from NAD to a modified histidine residue on elongation factor 2, thereby inhibiting protein synthesis which results in cell death (16). To test the ability of C32-delivered DNA encoding DT-A to inhibit protein synthesis in prostate cancer cells, we transfected LNCaP cells with C32-pCAG/luc and with a second C32 formulation, either C32-pRSV/FRT2PSA.FLP/EGFP or C32-pRSV/FRT2PSA.FLP/DT-A. Control cells were transfected with C32-pRSV/FRT2PSA.FLP/EGFP only.

pRSV/FRT2PSA.FLP/DT-A contains a coding sequence for Flp recombinase under control of the modified promoter of the PSA gene, PSA-BC, which has been previously described (14) and also contains an RSV promoter. The construct also contains a coding sequence for DT-A. However, the coding sequence for DT-A is not associated with an operably linked promoter, so no transcription can occur. The construct further contains two sites for Flp-mediated DNA recombination (FRT) positioned between the RSV promoter and the coding sequence for DT-A. Additional sequence containing a selectable marker is located between the FRT sites. This arrangement is such that recombination catalyzed by Flp results in removal of the intervening sequence between the two FRT sites, so that the RSV promoter is positioned upstream of and in close proximity to the DT-A coding sequence and directs its transcription. pRSV/FRT2PSA.FLP/EGFP is similar except that it contains a sequence coding for EGFP rather than one coding for DT-A.

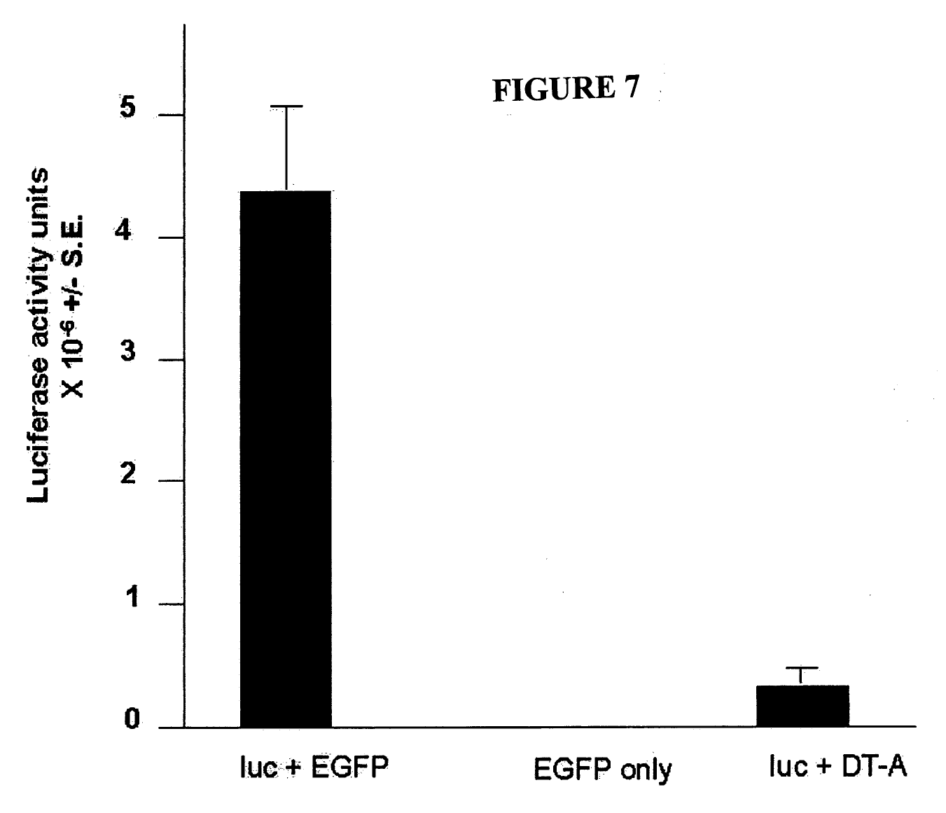

Forty-eight hours following transfection , we prepared protein extracts from cells and assayed luciferase activity. As shown in FIG. 7, luciferase activity in cells co-transfected with pCAG/luc and the DT-A construct was over 10× lower than in cells co-transfected with pCAG/luc the EGFP construct. These results demonstrate that following C32-delivery of a DNA construct in which DT-A expression is regulated both transcriptionally and by DNA recombination, DT-A expression in prostate cancer cells effectively inhibits protein synthesis.

### Example 3

### In Vivo DNA Delivery to Tumor Xenografts Causes Tumor Regression or Inhibits Tumor Growth

Materials and Methods

Plasmid construction. pCAG/luc plasmid DNA was constructed as described in Example 2.

Xenograft experiments. DNA (either naked or complexed to C32 or PEI) was administered to 8-week old nu/mu male mice (Harlan, Indianapolis, Ind.) by intratumoral (I.T.) injection. Mice were maintained under standard laboratory conditions. To complex plasmid DNA to C32, the polymer was dissolved in dimethyl sulfoxide (100 mg/ml). DNA (50 μg) was suspended in 25 μl 25 mM sodium acetate buffer, pH 5.0, and mixed with C32 polymer (300 or 1500 μg), also diluted in 25 μl 25 mM sodium acetate buffer, pH 5.0. After incubation of the polymer/DNA mixture at room temperature for 5 minutes, 10 μl 30% glucose in PBS was added to the 50 μl polymer/DNA mixture.

Plasmid DNA was complexed to Jet PEI® (Qbiogene, Montreal, Canada) according to the manufacturer's protocol for in vivo administration excepting that when 50 μg DNA was complexed, the volume was reduced to 60 μl instead of the recommended 400 μl. Uncomplexed pCAG/luc DNA (50 μg in 100 μl 5% glucose in 25 mM sodium acetate) was also administered to mice. For I.M. injections, a 28-gauge needle was used to deliver a 100 μl volume to the hind leg muscle. Xenografts, generated by subcutaneous injection of 5×10⁵ PC3 cells and 2×10⁶ LNCaP cells in PBS+20% Matrigel, were approximately 300 mm³ at the time DNA was administered. A 26-gauge needle was used to deliver a 60 μl volume to tumors. Calipers were used to measure the length and width of some tumors. Mice were sacrificed 2 days after I.T. injections, and 20 days after I.M. injections.

Results

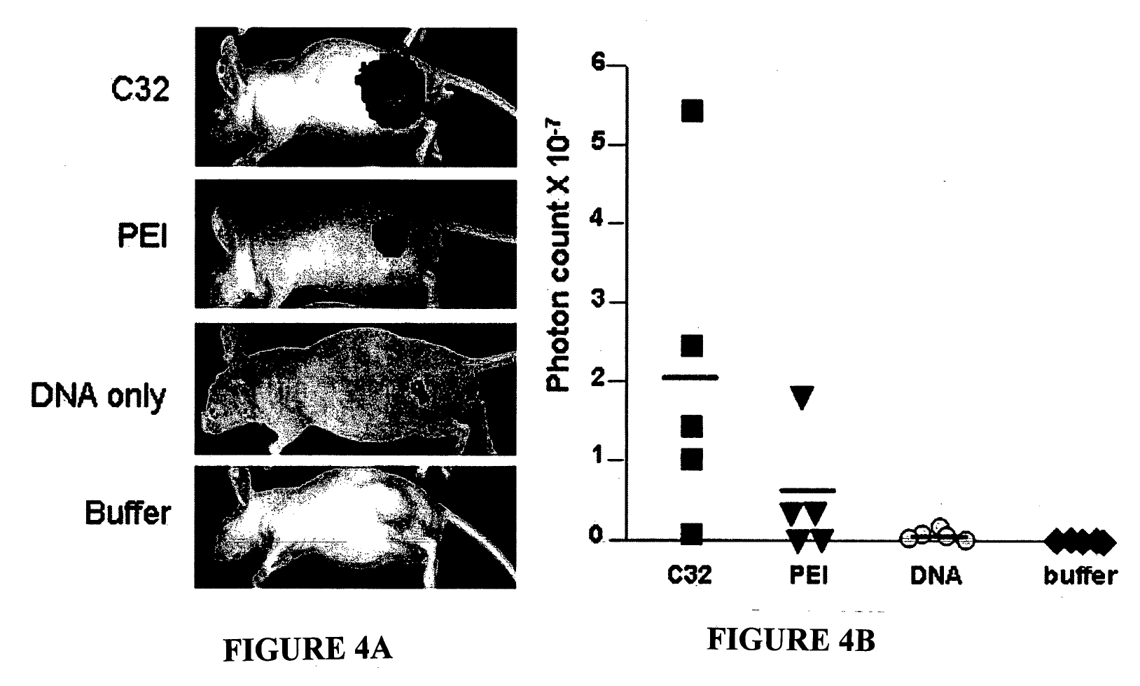

To test the utility of C32 for gene delivery in vivo, we examined transfection in a mouse xenograft model. PC3 human prostate tumor cells were mixed with Matrigel and inoculated subcutaneously into the flanks of nude mice to generate tumors. When tumor volumes were approximately 300 mm³, we injected C32-pCAG/luc nanoparticles intratumorally (50 μg DNA/injection, 30:1 polymer:DNA ratio). For comparison, we injected tumors with DNA complexed with in vivo Jet PEI®, the current state-of-the-art commercially-available transfection polymer, and with naked DNA. Control tumors were injected with 25 mM sodium acetate buffer, pH 5.0 (n=5 for each treatment group). Forty-eight hours after injection of DNA, we imaged mice and quantified bioluminescence using an IVIS® Bioluminescence Imaging System (FIG. 4). The average transfection mediated by C32 was 4-fold higher than transfection mediated by PEI, and 26-fold higher than transfection by naked DNA.

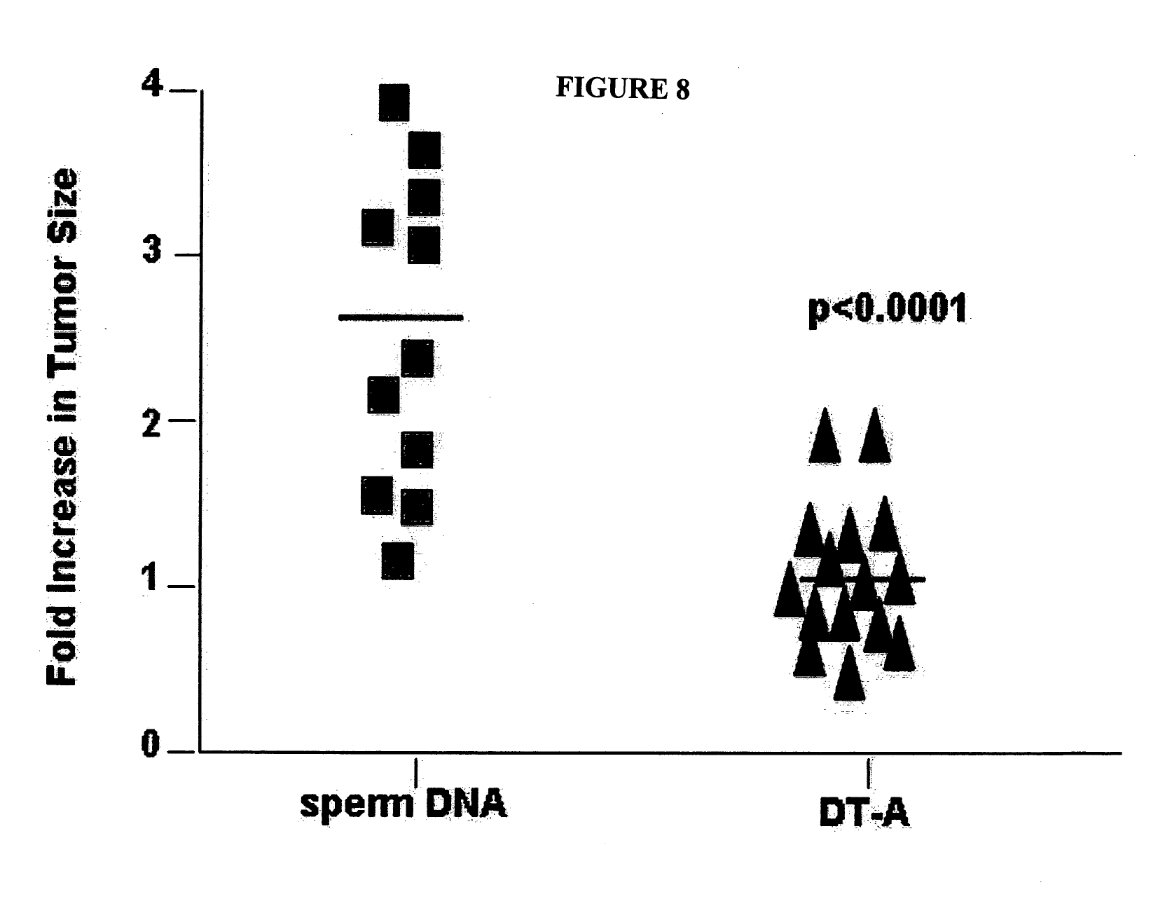

Having used a luciferase reporter construct to establish that C32 polymer can effectively transfer DNA to xenografts, we wished to determine whether C32-delivered DNA encoding DT-A would inhibit growth of tumor cells in vivo. LNCaP human prostate cancer cells were mixed with Matrigel and inoculated subcutaneously into the flanks of nude mice to generate tumors. When tumors attained a volume of approximately 100 mm³, we injected either C32-pRSV/FRT2PSA.FLP/DT-A or C32-salmon testes DNA intratumorally (50 μg DNA/injection, 30:1 polymer:DNA ratio). We administered C32/DT-A to tumors five more times, for a total of 6 injections over a period of 14 days. We used calipers to measure tumor size before the first injection, and on the final day, at which time mice were euthanized. The average growth rate of tumors injected with C32-pRSV/FRT2PSA.FLP/DT-A was suppressed 2-fold compared to control tumors (p<0.0001) (FIG. 8). In fact, 3/15 tumors treated with C32/DT-A failed to grow at all and 6/15 actually regressed in size. We conclude that expression of C32-delivered DT-A suppressed tumor growth and was capable of achieving tumor regression.

### Example 4

### In Vivo DNA Delivery to Muscle Tissue

Materials and Methods

pCAG/luc DNA (either naked or complexed to C32 or PEI) was prepared as described in Example 2 and administered to 8-week old nu/nu male mice (Harlan, Indianapolis, Ind.) by intramuscular (I.M.) injection using a 28-gauge needle was used to deliver a 100 μl volume to the hind leg muscle. Mice were maintained under standard laboratory conditions and were sacrificed 20 days after I.M. injections.

Results

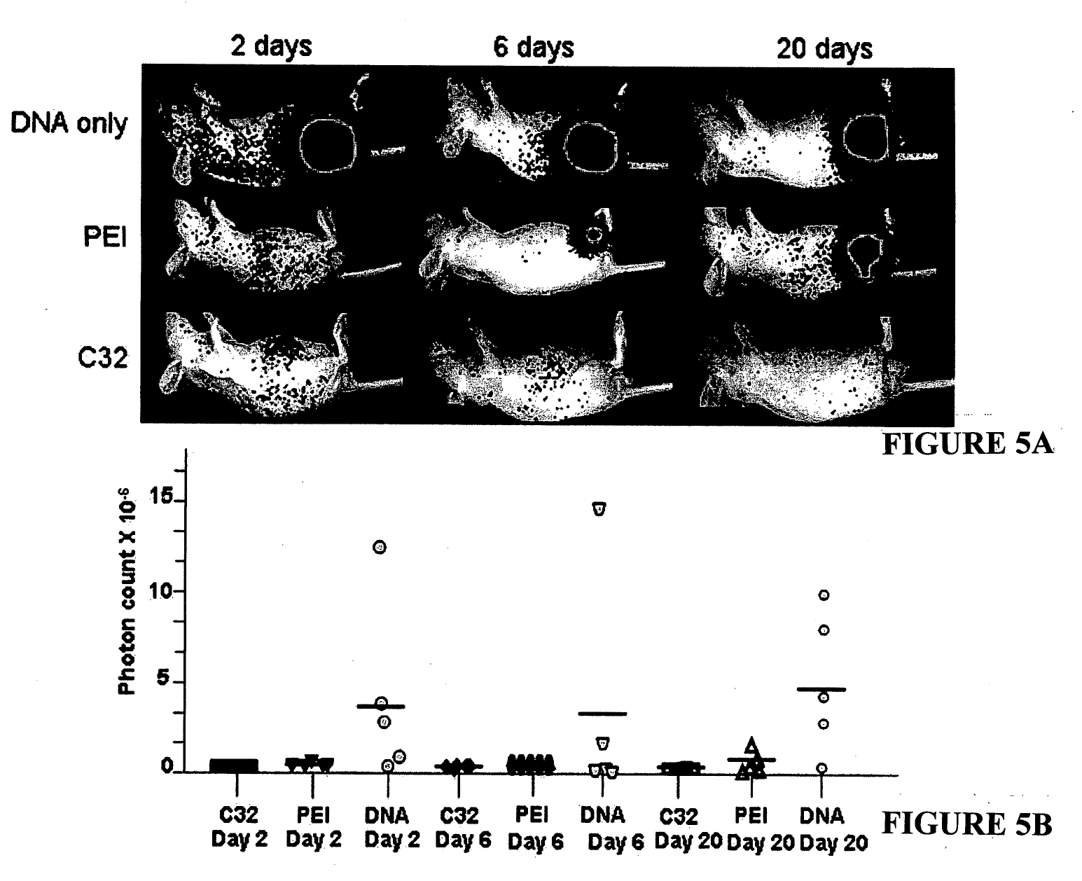

We measured transfection of healthy muscle using C32, PEI, and naked DNA (FIG. 5) (n=5 for each group). We injected complexed DNA or naked DNA into muscle and measured luciferase expression 2, 6, and 20 days following injection. Interestingly, transfection results were completely opposite that of intratumoral transfection. Naked DNA resulted in the highest levels of gene expression over the course of the experiment, PEI resulted in lower and delayed expression, and C32 did not result in any muscle transfection over the course of the experiment.

### Example 5

### In Vivo DNA Delivery to Various Tissues

Materials and Methods

Complex preparation and in vivo Administration. C32:DNA complexes containing 50 μg pCAG/Luc DNA were prepared as described in Example 2. Complexes were administered to 8-week old nu/nu male mice (Harlan, Indianapolis, Ind.) by injection. To administer the complexes, the tissue to be injected was exposed through an abdominal incision, and the tissue was injected with C32 polymer complexed to pCAG/Luc. The incision was closed with a surgical clip. Forty-eight hours after injection of DNA, we imaged mice and quantified bioluminescence using an IVIS® Bioluminescence Imaging System. The mice were then sacrificed and imaged again after opening the abdominal cavity. Various organs and tissues were removed and imaged.

Results

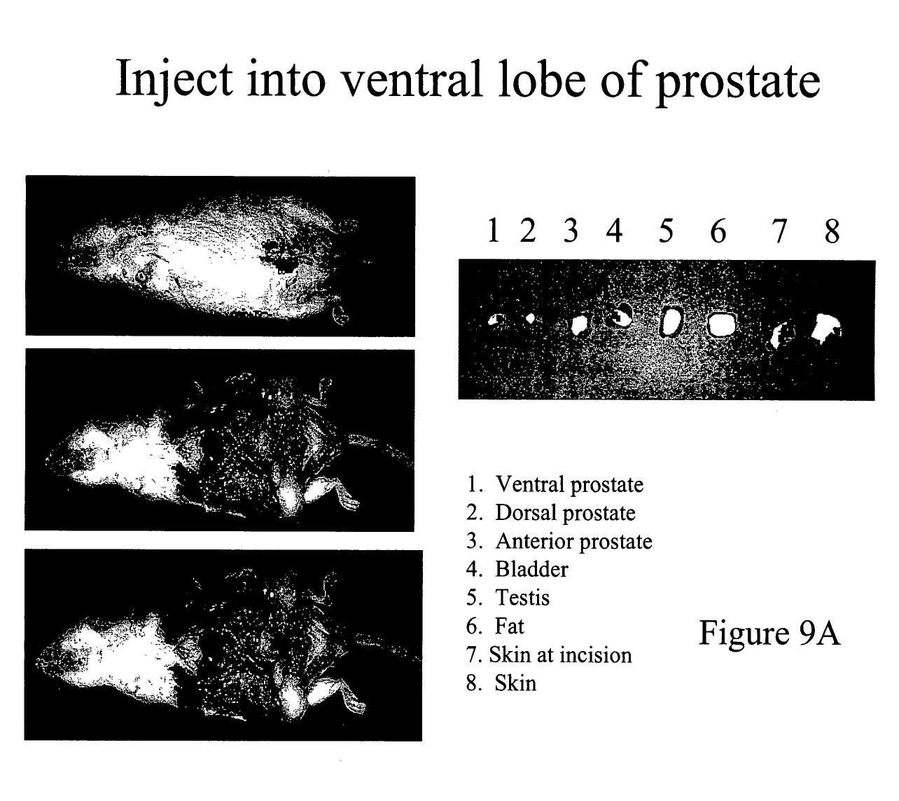

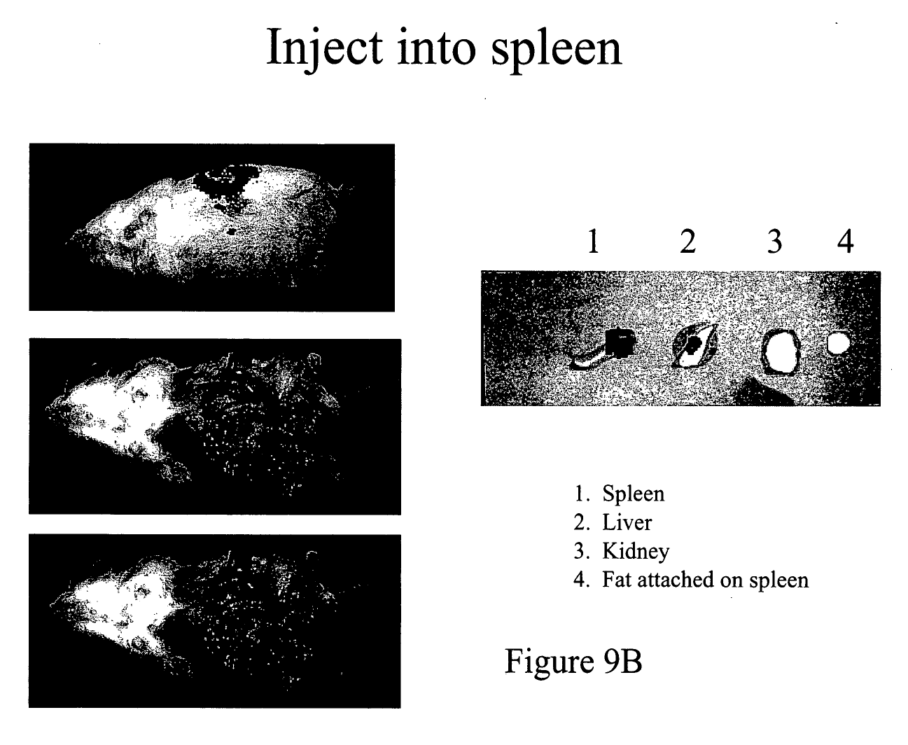

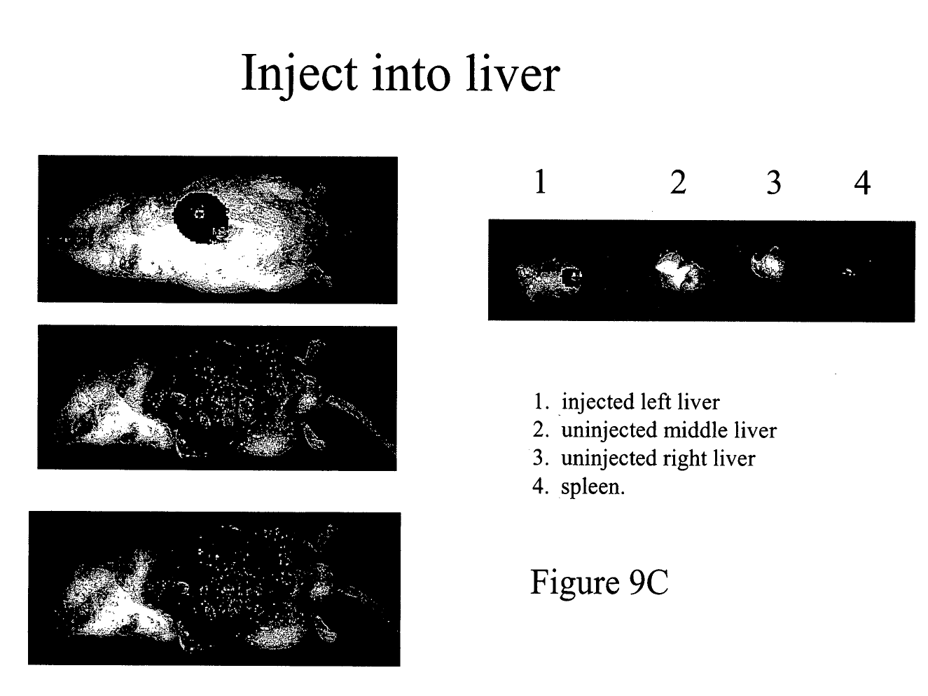

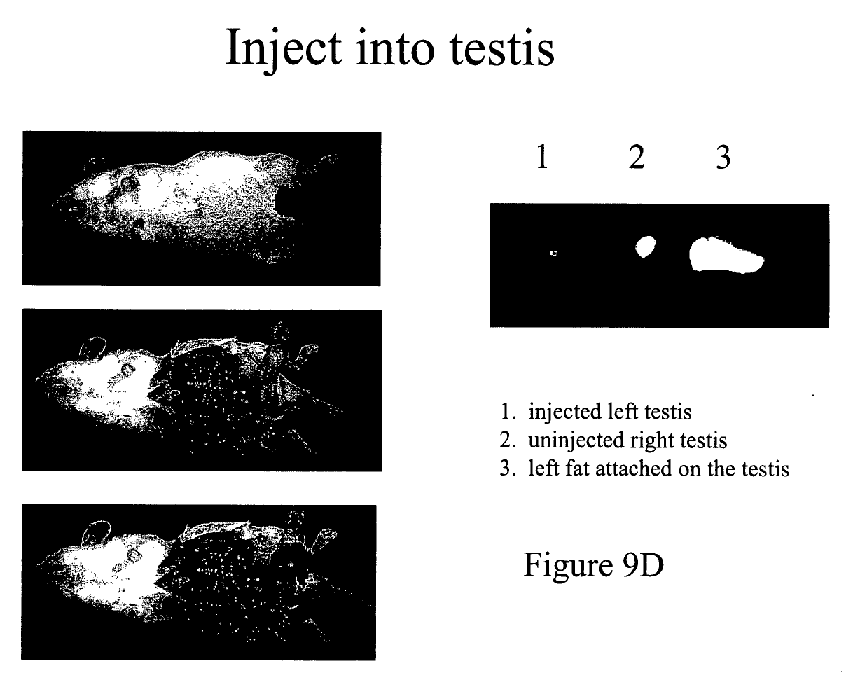

We examined transfection of a variety of other healthy tissues in addition to muscle. We injected complexed DNA into prostate, spleen, liver, and testis. Mice were imaged 48 hours later both prior to sacrifice and after opening of the abdominal cavity to reveal the injected organs and other tisues. Various organs and tissues were dissected and imaged. Results are shown in FIGS. 9A-9D. Each figure shows luminescence images prior to sacrifice (upper left panel) and after sacrifice (lower left panel). The figures also show luminescence images of various organs following dissection (right). As shown in the figures, robust expression was observed in each injected tissue. In some cases expression was also observed in tissues adjacent to those that were injected, possibly due to escape of complexes from the injected organ as a result of the relatively large injection volume in comparison to the size of some injected organs. Preliminary results suggest that some expression was also observed in various healthy tissues when the same DNA was similarly injected in buffer in the absence of polymer.

### Example 6

### Biocompatibility of Polymer Compositions

Materials and Methods

Histological analysis. Tumor or muscle samples were fixed in formalin, embedded in paraffin, sectioned and stained with hematoxylin and eosin according to standard procedures. The samples were thinly sectioned prior to embedding, and multiple levels were examined for each sample to minimize the possibility of not visualizing the injection site. Microscopic evaluation was performed on an Olympus BX41 microscope equipped with an Olympus Q-Color digital camera for image capture.

Blood analysis. A cardiac puncture was performed at sacrifice and serum was sent to an outside laboratory (LabCorp, Research Triangle Park, N.C.) for analysis of creatinine (Cr), total bilirubin (TBili), alkaline phosphatase (AlkPhos), alanine aminotransferase (ALT), gamma glutamyltransferase (GGT), lactate dehydrogenase (LDH) and creatine kinase (CK).

Results

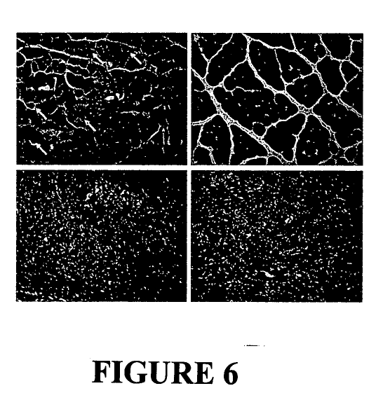

Histological sections of tumors injected with polymer:DNA complexes or with naked (uncomplexed) DNA, as described in Example 2, revealed a poorly differentiated carcinoma with occasional foci of necrosis and numerous mitotic figures within subcutaneous tissue; no histologic differences were observed between any of the groups. Histological sections of muscle injected with polymer:DNA complexes as described in Example 3 contained foci of calcification associated with myocyte nuclear internalization and atrophy, consistent with myocyte damage, at the site of injection of all of the PEI/DNA complexes (FIG. 6). Similar analysis of muscle injected with C32:DNA complexes and naked DNA demonstrated no pathology. No statistically significant differences were observed between intramuscular injection of C32:DNA, PEI:DNA, naked DNA or buffer in serum levels of markers of renal function (Cr), liver function (Tbili, AlkPhos, ALT, GGT, LDH) or muscle damage (CK) (data not shown).

### Example 7

### Shrinkage of Healthy Prostate Tissue Following Injection of C32 Polymer Complexed with PST/DT-A

Materials and Methods

Plasmid construction.

The plasmid pPSA/DT-A consists of a modified chimeric PSA promoter/enhancer sequence deriving from the plasmid pPSE-BC (gift of Lily Wu, UCLA) (14) and the coding sequence for the diphtheria toxin A chain, derived from the plasmid p22EDT1 (gift of A. Francis Stewart, EMBL, Heidelberg) (6).

The plasmid pMECA/DTA (see below) was digested with AgeI and SalI to release a 1.3 kb fragment containing DT-A coding sequence. This fragment was ligated to a 5.7 kb fragment derived from digestion of the plasmid pDC312/PSALucf (see below) with AgeI and SalI to create pPSA/DTA.

pMECA/DTA: A 1.3 kb fragment containing the DT-A coding sequence, obtained by digestion of the plasmid pIND/DTA (see below) with KpnI and XbaI, was ligated to KpnI+XbaI digested pMECA (15) to create pMECA/DTA.

pPSALucf: A 2.0 kb fragment containing the firefly luciferase coding sequence, obtained by digestion of the plasmid pMECA/Lucf (see below) with EcoRI and SalI, was ligated to EcoRI+SalI digested pDC312/PSA (see below) to create pPSA/Lucf.

pIND/DTA: A 1.3 kb fragment containing the DT-A coding sequence, obtained by digestion of the plasmid p22EDT1 (gift of A. Francis Stewart, EMBL, Heidelberg) with BglII and NotI, was ligated to BamHI+NotI digested pIND (Invitrogen) to create pIND/DTA.

pMECA/Lucf: A 2.0 kb fragment containing the firefly luciferase coding sequence, obtained by digestion of pBCVP2G5-lucNSN (gift of Lily Wu, UCLA) with BglII and SalI, was ligated to BglII+SalI digested pMECA (15) to create pMECA/LucF.

pDC312/PSA: A 2.5 kb fragment containing a modified chimeric PSA promoter/enhancer sequence, obtained by digestion of the plasmid pMECA/PSA (see below) with EcoRI, BglII, and EcoRI, was ligated to EcoRI+BamHI digested pDC312 (Microbix) to create pDC312/PSA.

pMECA/PSA: A 2.5 kb fragment containing a modified chimeric PSA promoter/enhancer sequence, obtained by digestion of the plasmid pPSE-BC (gift of Lily Wu, UCLA) with XbaI and SalI, was ligated to XbaI+SalI digested pMECA (15) to create pMECA/PSA.

Complex preparation and in vivo administration. Complexes were prepared as described in Example 2. DNA (50 μg) complexed to C32 was administered to an 8-week old nu/nu male mouse (Harlan, Indianapolis, Ind.) by injection. To administer the complexes, the prostate was exposed through an abdominal incision, and the right ventral (RV) lobe was injected with C32 polymer complexed to PSA/DT-A DNA. 50 μg DNA was delivered. The incision was closed with a surgical clip. Five days later, the mouse was sacrificed and the prostate was examined.

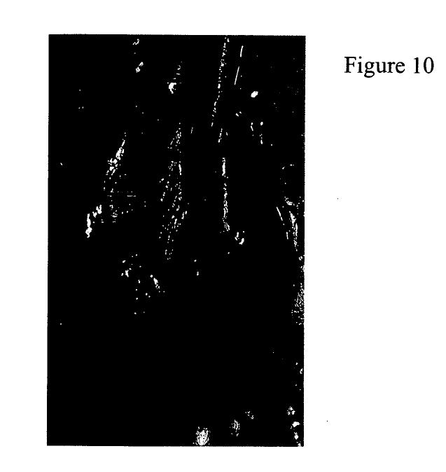

Results. We examined the ability of C32:PSA/DT-A complexes to destroy healthy mouse prostate tissue. The right ventral lobe of the prostate was injected with complex. The prostate gland was examined 5 days later. As shown in FIG. 10, the right ventral lobe was significantly reduced in size compared to the untreated left ventral (LV) lobe. The left lateral (LL) and right lateral (RL) lobes are of equal size. In control mice injected with C32 polymer complexed to PSA/Fluc DNA, which contains the same regulatory sequence of the human PSA gene controlling expression of firefly luciferase, RV and LV were of equal size (not shown).

### Example 8

### Higher Expression and Better Tissue Specificity following Intraprostatic Injection of Nanoparticle-Delivered DNA as Compared to Naked DNA

Materials and Methods.

Intraprostatic Injections in Mice. DNA (either naked or complexed to C32) was injected directly into the right ventral lobe of the prostate of 8-16 week old FVB/NJ male mice (Jackson Laboratory, Bar Harbor, Me.). DNA was complexed to C32 as described in Example 2. For intraprosatic injections, a small (˜1 cm) incision was made in the lower abdomen of anesthesized mice. An insulin syringe with a 28G needle was used to deliver a 60 μl volume to the right ventral lobe of the exposed prostate. The body wall was closed with a few stitches, and the wound site was closed with stainless steel surgical clips. All procedures performed on mice in this study were done in accordance with protocols approved by the Lankenau Institutional Animal Care and Use Committee.

Imaging Luciferase Activity. Optical imaging to detect luciferase activity in mice was performed using an IVIS® Bioluminescence Imaging System (113).

Results. We conducted additional experiments to further determine whether nanoparticle-delivered DNA is expressed specifically in mouse prostate following intraprostatic injection, we complexed C32 polymer with the DNA construct, pCAG/luc, encoding firefly luciferase, and injected the resulting nanoparticles directly into the prostate.

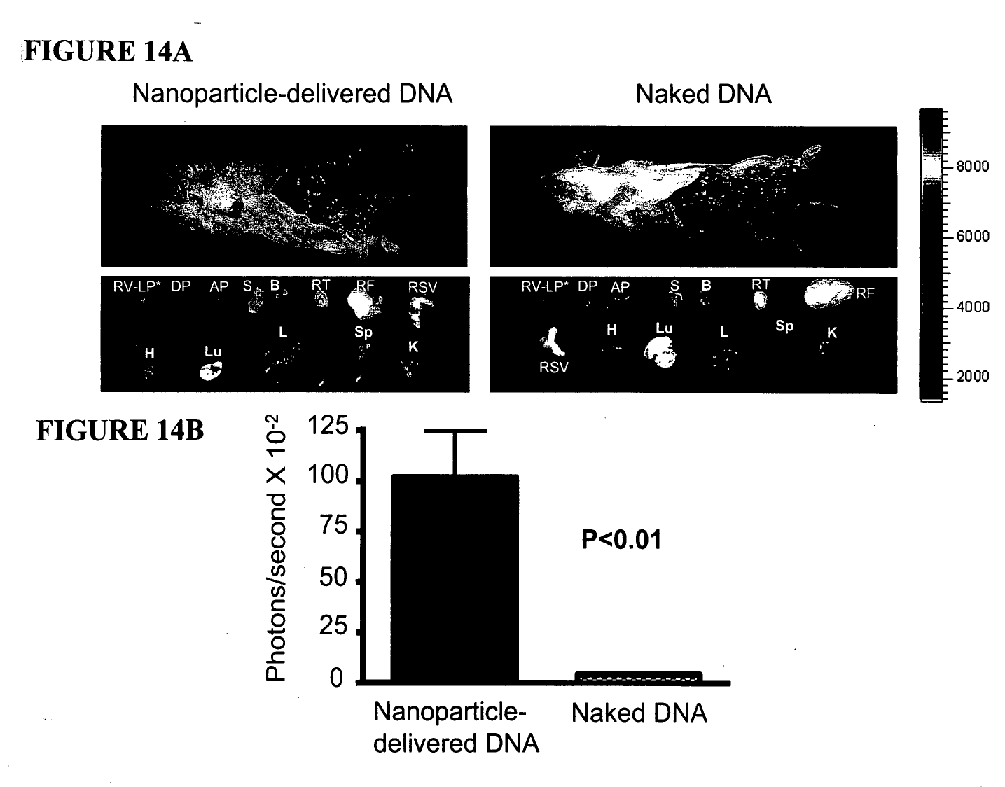

Following injection, we used whole mouse and ex vivo optical imaging to determine where luciferase was expressed. In the five mice we injected, luciferase activity was detected in the right ventral lobe of the prostate (the injected lobe) (5/5), as well as in the ventral skin overlying the injection site (4/5), bladder (2/5), fat (1/5), and seminal vesicle (1/5). No activity was detected in the dorsal and anterior lobes of the prostate, or in the testis, heart, lung, liver, spleen, and kidney (FIG. 14A, left). In contrast, when naked pCAG/luc DNA was injected, luciferase activity was only detected in the injected prostate lobe of one mouse (1/5). Activity was also observed in the bladder (3/5) and in the overlying skin (3/5) (FIG. 14A, right). No activity was observed in other organs and tissues. The observed expression in neighboring tissues most probably results from leakage of nanoparticles and naked DNA following intraprostatic injection. Expression of nanoparticle-delivered DNA was 10× higher than expression of naked DNA in the prostate, a significant difference (p<0.01) (FIG. 14B). Thus, compared to naked DNA, following intraprostatic injection, nanoparticle-delivered DNA expression was higher and more prostate-specific.

### Example 9

### Nanoparticle-Delivered PSA/DT-A DNA to Prostate Results in Gross Abnormalities in Prostate Morphology Resulting from Cellular Apoptosis

Materials and Methods.

Intraprostatic Injections in Mice. These were performed as described in Example 8.

Blood Analysis and Histology. A cardiac puncture was performed at killing, and serum was sent to an outside laboratory (LabCorp, Research Triangle Park, N.C.) for analysis of creatinine, total bilirubin, alkaline phosphatase, alanine aminotransferase, γ-glutamyl-transferase, lactate dehydrogenase, and creatine kinase. Multiple organs were collected, fixed in formalin for 2 hr, washed 3 times in PBS, and processed for paraffin embedding. 5 mm sections were prepared, H & E stained, and examined microscopically.

Results

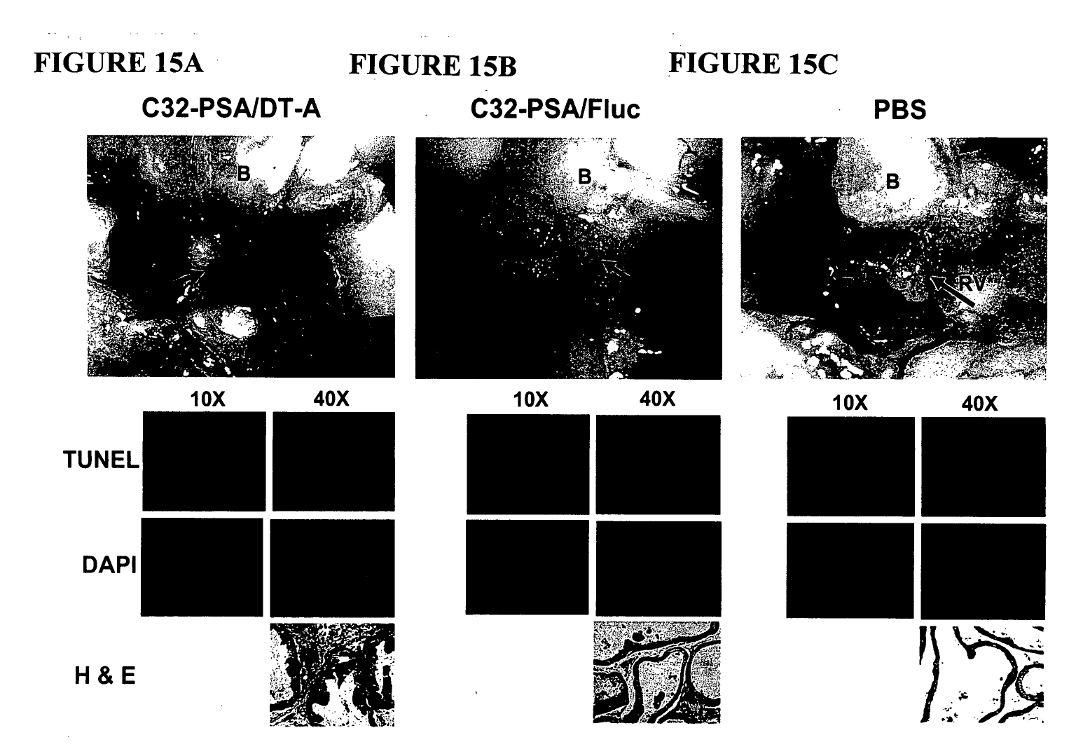

To extend the analysis of the effect of nanoparticle-delivered PSA/DT-A on the prostate described in Example 7, we next injected PSA/DT-A DNA, either as C32-nanoparticles or as naked DNA, directly into the right ventral lobe of mouse prostate. In this construct, a chimeric modified enhancer/promoter sequence of the human prostate-specific antigen (PSA) gene, PSE-BC, regulates the expression of DT-A. This promoter sequence is active discriminately in luminal cells in the mouse prostate, thus reflecting its activity in PSA-expressing cells in human prostate, as described above. Control mice were injected with PSA/Fluc nanoparticles or with phosphate buffered saline (PBS); some control mice underwent sham operations to expose the prostate, but were not injected. Mice in each group were sacrificed 3-7 days after injection, prostates were removed and examined using a dissecting microscope. We observed no difference in the distribution of abnormalities at different times post-injection. The results of this analysis are shown in Table I. We observed gross abnormalities in the appearance of 73% (11/15) of the prostates injected with DT-A nanoparticles. These abnormalities ranged from the presence of white opaque areas on the injected lobe (27%), reduction in size of the injected lobe (33%), or total ablation of the injected lobe (13%) (see FIG. 15). In contrast, all lobes (5/5) injected with naked PSA/DT-A DNA appeared normal as did lobes injected with PBS and uninjected lobes. Only eight percent ( 1/12) lobes injected with PSA/Fluc DNA had a visible gross abnormality (Table I). The variability we observed in the morphology of PSA/DT-A-injected prostates most likely reflects the technical difficulty associated with the injection procedure in the mouse model. These observations demonstrate that nanoparticle-delivered DNA encoding DT-A can result in gross changes in prostate morphology, including total ablation.

As described above, DT-A catalyzes the ADP-ribosylation of EF-2 elongation factor, an essential component for eukaryotic protein synthesis. As a result, the toxin kills most cells by causing apoptosis (111). To further characterize the effects of injecting PSA/DT-A nanoparticles into the mouse prostate, we used a TUNEL assay to assess the degree of apoptosis in histological sections of injected ventral lobes that displayed gross morphological abnormalities. We observed extensive numbers of apoptotic cells in the luminal compartment of the prostatic epithelium, as well as within the acini lumen themselves (FIG. 15A), while there was no evidence of increased number of apoptotic smooth muscle cells and other stromal cells in inter-acini spaces. As a result of epithelial cell death, the normal organization of the acini was severely disrupted. In contrast, we observed very few apoptotic cells and normal acini organization in prostates injected with PSA/Fluc nanoparticles or PBS (FIG. 15B & C). We also performed TUNEL assays on prostates injected with naked PSA/DT-A DNA. As with PBS-injected prostates, there were very few apoptotic cells and acini organization was normal (data not shown). Cell death in surrounding tissues and organs following intraprostatic injection of PSA/DT-A nanoparticles did not appear to increase above the low levels that normally occur (data not shown).

No statistically significant differences were observed between PSA/DT-A nanoparticle-injected and PBS injected mice in serum levels of markers of renal function (creatinine), liver function (total bilirubin, alkaline phosphatase, alanine aminotransferase, γ-glutamyltransferase, and lactate dehydrogenase), or muscle damage (creatine kinase) (data not shown). Histological analyses were performed on multiple organs from mice injected with PSA/DT-A nanoparticles. Organs analyzed include bladder, testis, epididymus, small intestine, large intestine, liver, spleen, pancreas, kidney, adrenal glands, lungs, thyroid, heart, skeletal muscle, skin, bone with marrow, and brain. No abnormalities were observed (data not shown). These results show that there is no toxicity beyond the confines of the prostate itself following intraprostatic injection of PSA/DT-A nanoparticles.

In summary, the results described in Examples 8 and 9 show that direct injection of polymeric nanoparticles to deliver DNA encoding diphtheria toxin to prostate cells resulted in a high incidence of apoptotic cells and reduction in the size of the prostate, with no effect on neighboring tissues. Furthermore, serology and histology of multiple organs from mice following intraprostatic injection of PSA/DT-A nanoparticles failed to identify any sign of systemic metabolic dysfunction or tissue pathology, indicating that the effects of the toxin are confined to the prostate. Direct injection of naked DNA into the prostate resulted in poor expression. This is in contrast to efficient expression of naked DNA upon intra-muscular injection as described above and in (106). Our results thus demonstrate that complexation of DNA with polymer enhances DNA delivery to prostate cells.

TABLE I
Effect of injection of either DNA-nanoparticles
or naked DNA into ventral prostate lobe^{A}.

| | Gross Abnormalities in Morphology of | | | | | | | |
|---|---|---|---|---|---|---|---|---|
| | Injected Prostate Lobe | | | | | | | |
| | | | | | totally | | Normal | |
| Treatment | white spot | | smaller | | ablated | | Appearance | |
| C32-PSA/DT-A | 4/15 | (26.7)^{B} | 5/15 | (33.3) | 2/15 | (13.3) | 4/15 | (26.7) |
| PSA/DT-A | 0/6 | (0) | 1/6 | (16.7) | 0/6 | (0) | 5/6 | (83.3) |
| naked DNA | | | | | | | | |
| C32-PSA/Luc | 1/12 | (8.3) | 0/12 | (0) | 0/12 | (0) | 11/12 | (91.7) |
| PBS | 1/7 | (14) | 0/7 | (0) | 0/7 | (0) | 6/7 | (86) |
| No treatment | 0/4 | (0) | 0/4 | (0) | 0/4 | (0) | 4/4 | (100) |

^{A}Data summarizes results of three separate experiments. Mice were sacrificed 3-7 days after injection.
^{B}Numbers in parentheses, percentage

### Example 10

### Nanoparticle-Delivered PSA/DT-A DNA Specifically Kills Luminal Cells in the Prostatic Epithelium

Materials and Methods.

DNA Constructs. To construct pPSA/EGFP, the plasmid PSE-BC was digested with XbaI and SalI. The resulting 2.5 kb fragment, containing the PSE-BC promoter/enhancer sequence, was ligated to (XbaI+SalI)-digested pMECA to generate pMECA-PSA. pMECA-PSA was digested with SalI and BglII, and then with XmnI. The resulting 2.5 kb fragment was ligated to (SalI+BglII)-digested pEGFP-1 (Clontech, Mountain View, Calif.) to generate pPSA/EGFP.

To construct pK5/ECFP, the plasmid pECFP (Clontech) was digested with BamHI and AflII. The resulting 1 kb fragment containing the CFP sequence was ligated to (BamHI+AflII)-digested pIND (Stratagene, LaJolla, Calif.) to create pIND/ECFP. pIND/ECFP was digested with BamHI and NheI. The resulting 1 kb fragment was ligated to (BglII+NheI)-digested pMECA to create pMECA-ECFP. A 7 kb fragment, released by KpnI-digestion of the plasmid p3/4 (gift of the Deutsches Krebsforschungzentrum, Heidelberg, Germany), was digested with SalI. The resulting 5.2 kb fragment containing the K5 promoter sequence was ligated to (KpnI+XhoI)-digested pMECA-ECFP to create pK5/CFP.

Transgenic Mice. pK5/CFP was digested with NheI and KpnI and the resulting 6.2 kb transgene fragment, containing the keratin 5 promoter and the cyan fluorescent protein (CFP) was purified and microinjected into B6C3F2 fertilized mouse oocytes as described (Hogan, B., Constantini, F., and Lacy, E., Manipulating the Mouse Embryo. A Laboratory Manual. Cold Spring Harbor Laboratory, Cold Spring Harbor, N.Y., 1986.) PSA/EGFP transgenic mice were generated using a 3.5 kb transgene fragment derived from digestion of pPSA/EGFP with BglII and AflII.

Human Primary Prostate Cell Lines. Human primary prostate cell lines PrEC (epithelial; express cytokeratins 8 and 13), PrSc (stromal; express vimentin, but not cytokeratins), and PrSMC (smooth muscle; express β-actin) (Cambrex, East Rutherford, N.J.) were grown in their respective Bullet kit mediums (Cambrex) at 37° C. in 5% CO₂, balance air.

Apoptosis and Cell Death Assays After fixation in formalin for 2 hr, tumors and prostates were processed for paraffin embedding. 5 μm sections were prepared; some were H & E stained, while apoptotic cells were identified in others by TUNEL assay using an In Situ Death Detection Kit (Roche Boehringer Mannheim, Indianapolis, Ind.) according to the manufacturer's protocol. Cover slips were mounted using Vectashield Mounting Medium for fluorescence with DAPI (Vector Laboratories, Inc., Burlingame, Calif.) A Zeiss Axiovert fluorescent microscope was used for viewing stained cells. Statistical comparison of the numbers of TUNEL positive cells treated with DT-A and LUC nanoparticles was made using an unpaired two-tailed student t test.

Reporter Gene and Protein Assays. We used the Luciferase Assay System (Promega, Madison, Wis., USA) and a Monolight 2010 luminometer (Analytical Luminescence Laboratory, San Diego, Calif., USA) to measure luciferase activity in cell extracts that were prepared according to the manufacturer's instructions. Total protein in cell extracts was measured using a BCA Protein Assay Kit (Pierce, Rockford, Ill.) according to manufacturer's instructions. To observe CFP and GFP fluorescence in transgenic mouse prostates, prostates were fixed in 4% paraformaldehyde for 30 min at room temperature, washed 3 times with phosphate buffered saline, and mounted in OCT for frozen sectioning. Frozen sections were observed using a Zeiss Axioplan fluorescent microscope equipped with CFP and GFP filter sets and an Axiocam camera.

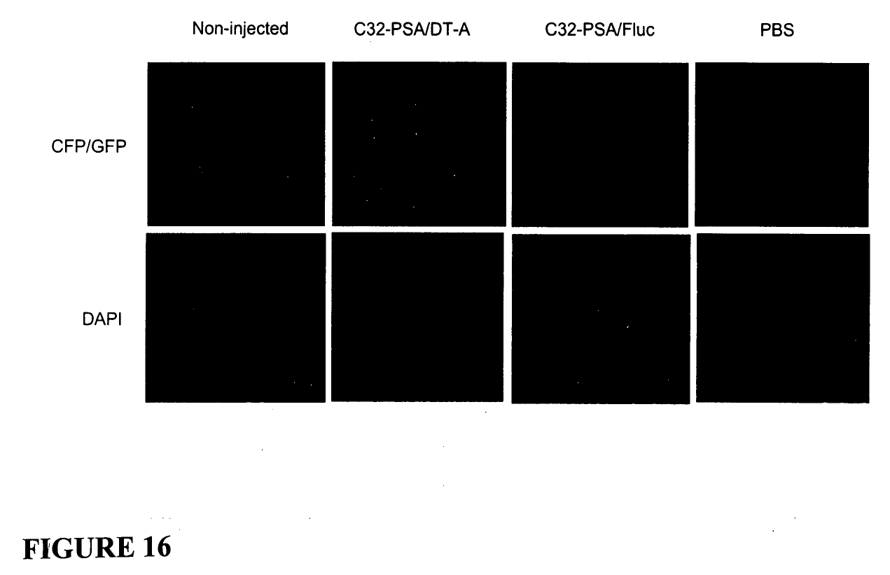

Results. To further explore the specificity with which nanoparticle-delivered PSA/DT-A DNA kills cells following intraprostatic injection, we generated two transgenic mouse lines, PSA/EGFP and K5/CFP. In PSA/EGFP mice, the chimeric PSA promoter/enhancer PSE-BC (see above) targets expression of green fluorescent protein (EGFP) to luminal cells in the prostatic epithelium, while in K5/CFP mice, the cytokeratin 5 promoter targets expression of cyan fluorescent protein (CFP) to basal cells in the epithelium of various organs, including the prostate (FIG. 16). We crossed PSA/EGFP mice with K5/CFP mice to generate double transgenic mice, and then injected PSA/DT-A nanoparticles directly into the right ventral prostatic lobe of these mice. Mice were sacrificed 5 days post-injection and frozen sections of prostates with gross morphological abnormalities (opaque areas) were prepared and viewed using a fluorescent microscope. We observed a reduction in GFP expression in PSA/DT-A injected lobes of double transgenic mice, reflecting shut-down of protein synthesis in PSA-expressing luminal cells (FIG. 16; enlarged images are available on-line). In control mice injected with PSA/Fluc nanoparticles, GFP expression was not different from that observed in mice injected with PBS or in non-injected mice (FIG. 16). CFP expression in lobes injected with either PSA/DT-A or PSA/Fluc nanoparticles was similar. These results are further evidence that the PSA regulatory sequence effectively targets DT-A expression to luminal cells, resulting in their death.

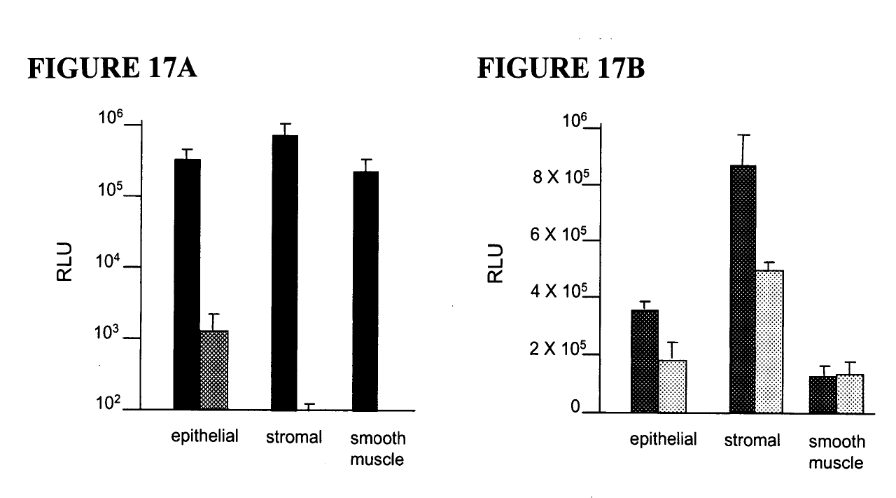

To determine whether PSA/DT-A nanoparticles kill human prostate cells with the same specificity observed in mice, we first transfected three different human primary cell lines of prostatic origin (epithelial, stromal, and smooth muscle) with C32-PSA/Fluc nanoparticles to confirm that the PSA promoter is active specifically in epithelial cells (FIG. 17A). Luciferase activity was 10× higher in epithelial cells as compared to stromal cells in which activity was just above the background level. No activity was detected in smooth muscle cells. We next transfected cells with C32-CAG/Fluc nanoparticles, followed 3 hr later by C32-PSA/DT-A or by control nanoparticles (C32-PSA/Flp). After 48 hr, we measured luciferase enzyme activity, an assay for the inhibition of protein synthesis by DT-A in transfected cells. In both epithelial and stromal cells, luciferase activity was ˜50% lower in cells treated with the DT-A DNA as compared to cells treated with the control DNA, while there was no difference in activity between DT-A-treated and control-treated smooth muscle cells (FIG. 17B). The observed reduction in luciferase activity in stromal cells, despite the low activity of the PSA promoter in these cells, probably reflects the potency of the DT-A toxin. These results suggest that the PSA/DT-A DNA construct we injected into mouse prostate in this study will effectively bring about the death of human prostate epithelial cells, and perhaps stromal cells as well, following nanoparticle delivery of the DNA directly to an enlarged prostate.

Hyperproliferation of luminal cells, as well as stromal cells, leads to enlargement of the prostate with its associated ill effects. Our results show that the PSA promoter/enhancer we used is active in both luminal and stromal cells in culture, with activity in luminal cells being ˜10-fold higher than in stromal cells. Thus, this regulatory sequence targets gene expression to those cells that contribute to the development of BPH. In addition, use of this promoter ensures that cells in other tissues neighboring the prostate (e.g., bladder and urethra) are not killed by the toxin. Furthermore, evidence that PSA plays a role in stimulating the growth of prostatic stromal cells by modulating the availability of IGF-I (112) suggests that in the absence of PSA-producing luminal cells, stromal cell hyperproliferation will cease.

We note that a mouse model of the abnormalities associated with BPH does not exist, so in these studies, we injected nanoparticles into the normal prostate of mice. It would be expected that the apoptotic effect of the toxin would be similar in hyperproliferative luminal cells in BPH.

### References

1. American Urological Association Guideline on the Management of Benign Prostatic Hyperplasia, American Urological Association, 2003. (available at www.auanet.org/guidelines/bph.cfm)
2. Greenfield, L., Bjorn, M. J., Horn, G., Fong, D., Buck, G. A., Collier, R. J. & Kaplan, D. A. (1983) Proc. Natl. Acad. Sci. USA 80, 6853-6857.
3. Maxwell, I. H., Maxwell, F. & Glode, L. M. (1986) Cancer Res. 46, 4660-4664.
4. Yamaizumi, M., Mekada, E., Uchida, T. & Okada, Y. (1978) Cell 15, 245-250.
5. Peng, W., Verbitsky, A., Bao, Y. & Sawicki, J. A. (2002) Mol. Ther. 6, 537-545.
6. Angrand, P.-O., Woodroofe, C. P., Buchholz, F. & Stewart, A. F. (1998) Nucl. Acids Res. 26, 3263-3269.
7. Lee, E. J. & Jameson, J. L. (2002) Human Gene Ther. 13, 533-542.
8. Luo, D. & Saltzman, W. M. (2000) Nat. Biotechnol. 18, 33-37.
9. Wang, J., Zhang, P. C., Mao, H. Q. & Leong, K. W. (2002) Gene Ther. 9, 1254-1261.
10. Ledley, F. D. (1995) Human Gene Ther. 6, 1129-1144.
11. Anderson, D. G., Lynn, D. M. & Langer, R. (2003) Angew. Chem. Int. Ed. Engl. 42, 3153-3158.
12. Akinc, A. B., Anderson, D. G., Lynn, D. M. & Langer, R. (2003) Bioconjugate Chem. 14, 979-988.
13. Schaffer, D. V., Fidelman, N. A., Dan, N. & Lauffenburger, D. A. (2000) Biotechnol. Bioeng. 67, 598-606.
14. Wu, L., Matherly, J., Smallwood, A., Adams, J. Y., Billick, E., Belldegrun, A. & Carey, M. (2001) Gene Ther. 8, 1416-1426.
15. Thomson, J. M. & Parrott, W. A. (1998) BioTechniques 24, 922-928.
16. Collier, R. J. (1975) Bacteriol. Rev. 39, 54-85.
17. Greenberg, N. M., DeMayo, F., Finegold, M. J., Medina, D., Tilley, W. D., Aspinall, J. O., Cunha, G. R., Donjacour, A. A., Matusik, R. J. & Rosen, J. M. (1995) Proc. Natl. Acad Sci. U.S.A. 92, 3439-3443.
18. Barry, M. J., et al. (1992) J. Urol. 148:1549-1557.
19. Barry, M. J., et al., (1992) J. Urol. 148:1558-1563.
20. Sadowski P. (1986) J Bacteriol. 165(2):341-7.
21. Gorman, C. and Bullock, C. (2000) Curr Opin Biotechnol.11(5):455-60.
22. Kuhn R, Torres R M. (2002) Methods Mol Biol. 180:175-204.
23. Sadowski P D. (1995) Prog Nucleic Acid Res Mol Biol. 51:53-91.
24. Niwa H. et al. (1991). Gene 108(2):193-9
25. Okabe M. et al. (1997). FEBS Lett. 407(3):313-9.
26. Xu Z L. et al. (2001). Gene 272:149-156
27. Kim D W et al. (1990). Gene 91(2):217-23
28. Guo Z S et al. (1996). Gene Ther. 3(9):802-10.
29. Adra C N. et al. (1987). Gene 60(1):65-74
30. Gerolami R. et al. (2000). Cancer Gene Ther 7(9):1286-92
31. Sugiyama H. et al. (1988) Gene 65(1):135-9.
32. Muller S R. et al. (1990) DNA Cell Biol 9(3):221-9.
33. Yew N S et al. (2001) Mol Ther. 4(1):75-82.
34. Schorpp, M., et al., (1996) Nucleic Acids Res. 124(9):1787-8.
35. Latham, JP, et al.(2000) Cancer Res., 60:334-341.
36. Watt, F., et al., (2001) Genomics. 73(3):243-54.
37. Ikegami, S., et al., (2002) Jpn J Cancer Res. 93(10): 1154-63.
38. Uchida, A., et al., (2001) Urology. 58(2 Suppl 1):132-9.
39. Zhang, J., et al., (2004) Endocrinology. 145(1):134-48.
40. Gabril, M Y, et al., (2002) Gene Ther. 9(23):1589-99.
41. Cheng, W S, et al., Mol Ther. 10(2):355-64.
42. Jain, A., et al., (2002) Mol Endocrinol. 16(10):2323-37.
43. Kitazono, M., et al., (2002) Int. J Cancer 99(3):453-9.
44. Takeda, T., et al., (2002) Cancer Gene Ther. 9(10):864-74.
45. Yamazaki, M., et al., (2004) Cancer Gene Ther. 11(1):8-15.
46. Puppin, C. et al., (2004) Mol Cell Endocrinol. 214(1-2):117-25.
47. Linder, K, et al., (2004) J Lipid Res. 45(1):148-54.
48. Lopez, I P, et al., (2003) Obes Res. 11 (2):188-94.
49. Combs, T P, et al., (2003) Diabetes 52(2):268-76.
50. Villena, J A, et al., (2004) J Biol Chem. Aug 27 ePub ahead of print.
51. Wilcox, A., et al. (2004) J Biol Chem. 279(37):38881-8.
52. Owens, D. W., and Iane, E. B., (2003) BioEssays 25:748-758.
53. Skubitz, K M. and Skubitz, A P. (2003) J Lab Clin Med. 141(5):297-308.
54. Kasper, S. and Smith, J A. (2004) J Urol. 172:12-19.
55. Cooper, S. (2003) The Lancet 362:459-69.
56. Bahn, R. S. (2003) J Clin. Endocrinol & Metab. 88(5):1939-1946.
57. Plante, M. K., et al. (2004) J. Urol. 172:20-26.
58. Goya, N., et al., (1999) J. Urol. 162:383-386.
59. Ditrolio, J. et al. (2002) J. Urol. 167:2100-2104.
60. Allen, T. M. (2002) Nature Reviews Cancer, 2, 750-763.
61. Collier, R. J. (1975) Bacteriol Rev. 39:54-85.
62. Yamaizumi, M. (1978) Cell, 15:245-250.
63. Maxwell, I H, et al. (1986) Cancer Res., 46:4660-4664.
64. Doubrow, M., Ed., “Microcapsules and Nanoparticles in Medicine and Pharmacy,” CRC Press, Boca Raton, 1992;
65. Mathiowitz and Langer, (1987) J Controlled Release 5:13-22.
66. Mathiowitz et al. (1987) Reactive Polymers 6:275-283.
67. Mathiowitz et al. (1988) J. Appl. Polymer Sci. 35:755-774.
68. Cotten et al. (1993) Methods Enzym. 217:618.
69. Ledley, F D, et al. (1995) Human Gene Ther 6:1129-1144.
70. Luo, D. et al. (2000) Nat. Biotechnol., 18:33-37.
71. Lynn, D. M.; Anderson, D. G.; Putnam, D.; Langer, R. (2001) J Am Chem Soc 123, 8155-8156.
72. Akinc, A.; Lynn, D. M.; Anderson, D. G.; Langer, R. (2003) J Am Chem Soc 125, 5316-5323.
73. Lim, Y. B.; Kim, C. H.; Kim, K.; Kim, S. W.; Park, J. S. (2000) J Am Chem Soc 122: 6524-6525.
74. Lim, Y. B.; Kim, S. M.; Suh, H.; Park, J. S. (2002) Bioconjugate Chemistry 13, 952-957.
75. Gebhart C L, Kabanov A V. (2001) J Control Release. 15;73(2-3):401-16.
76. Fominaya, J.; Wels, W. (1996) J Biol Chem 271, 10560-10568.
77. Schwartz, J. J.; Zhang, S. (2000) Curr Opin Mol Ther 2, 162-167.
78. Boussif, O.; et al., (1995) P Natl Acad Sci USA 92, 7297-7301.
79. Pannier, A K and Shea, L D (2004) Mol. Ther. 10(1):19-26.
80. Lynn, D. M., et al. (2000) J. Am. Chem. Soc., 122:10751-10768
81. Kabanov, A V, et al. (1998) Self-assembling complexes for gene delivery: from laboratory to clinical trial, Wiley, Chichester, N.Y.
82. Trubetskoy et al. (2003) Gene Therapy 10:261-271.
83. Kootstra, N A and Verma, I N, (2004) Annu Rev. Pharmacol Toxicol., 43:413-39.
84. McTaggart, S. and Al-Rubeai, M. (2002) Biotechnol. Adv. 20(1); 1-31.
85. Quinonez, R. and Sutton, R E, (2002) DNA Cell Biol. 21(12):937-51.
86. Sandrin, V., et al., (2003) Curr Top Microbiol Immunol 281:137-78.
87. Burton, E A, et al, (2002) DNA Cell Biol. 21(12):915-36.
88. Park, H S, et al. (2003) Gene Ther. 10(13):1129-34.
89. St. George, J A, (2003) Gene Ther. 10(14):1135-41.
90. Buning, H., et al. (2004) Cells Tissues Organs 177(3):139-50.
91. Guo Z S, Bartlett D L. (2004) Expert Opin Biol Ther. 4(6):901-17.
92. Papadakis, E D, et al. (2004) Curr Gene Ther. 4(1):89-113.
93. Peppas, N A, et al., (2000) Eur J Pharm Biopharm. 50(1):27-46.
94. Megeed, Z., et al., (2002) Pharm Res. 19(7):954-9.
95. Liu, A. Y. and True, L. D. (2002) Am. J. Path. 160: 37-43.
96. Liu, Y., et al, (1995), J. Biol. Chem. 270(42): 24864-24870.
97. Crook K, et al., (1998), Gene Ther. 5:137-143.
98. Kikuchi A, et al., (1999) Hum Gene Ther 10:947-955.
99. Serikawa T, et al, (2000), Biochim Biophys Acta, 1467:419-430.
100. Hansen C B, et al., (1995) Biochim Biophys Acta. 1239(2):133-44.
101. Torchilin V P, et al., (2001) Biochim Biophys Acta, 1511(2):397-411.
102. Ishida T, et al., (1999) FEBS Lett. 460(1):129-33.
103. C. H. Ahn, et al., (2002) J Control Release 80, 273-82.
104. Kichler, A., et al., (2001) J Gene Med. 3(2):135-44.
105. Brissault B, et al., (2003) Bioconjug Chem. 14(3):581-7.
106. Anderson, D. G., Peng, W., Akinc, A. B., Houssain, N., Kohn, A., Padera, R., Langer, R., and Sawicki, J. A. 2004. Proc. Natl. Acad. Sci. USA 101:16028-16033.
107. Iyer, M., Salazar, F. B., Lewis, X., Zhang, L., Carey, M., Wu, L., and Gambhr, S. S.
2004. Mol. Ther. 10:545-552.
108. Bao, Y., Peng, W., Verbitsky, A., Chen, J., Wu, L., Rauen, K. A., and Sawicki, J. A. 2005. Prostate 64:401-407.
109. Moehring, T. J., Danley, D. E., and Moehring, J. M. 1984. Mol. Cell Biol 4:642-650.
110. Kohno, K., Uchida, T., Ohkubo, H., Nakanishi, S., Nakanishi, T., Fukui, T., Ohtsuka, E., Ikehara, M., and Okada, Y. 1986. Proc. Natl. Acad Sci. USA 83:4978-4982.
111. Morimoto, H., and Bonavida, B. 1992. J. Immunol. 149:2089-2094.
112. Sutkowski, D. M., Goode, R. L., Baniel, J., Teater, C., Cohen, P., McNulty, A. M., SHsiung, H. M., Becker, G. W., and Neubauer, B. L. 1999. J. Natl. Cancer Inst. 91:1663-1669.
113. Peng, W., Chen, J., Hunag, Y.-H., and Sawicki, J. A. 2005. Gene Ther., published online on 30 Jun. 2005; doi:10.1038/sj.gt.3302580.

We claim:

1. A method for treating a disease or condition characterized by inappropriate or excessive noncancerous growth comprising the steps of:
providing a subject in need of treatment for a disease or condition characterized by inappropriate or excessive noncancerous growth of a tissue; and
administering a tissue-selective therapeutic composition comprising a therapeutic agent to the subject in an amount effective to cause a reduction in the size of the tissue or to inhibit continued increase in size of the tissue, wherein the composition does not comprise a viral delivery vehicle, and wherein the composition (i) comprises a tissue-selective delivery vehicle; (ii) comprises a polynucleotide; (iii) comprises both a tissue-selective delivery vehicle and a polynucleotide; (iv) is locally delivered; or (v) any combination of (i)-(iv).

2. The method of claim 1, wherein the step of administering the composition comprises locally administering the composition at or in the vicinity of a site of inappropriate or excessive noncancerous tissue growth.

3. (canceled)

4. (canceled)

5. (canceled)

6. (canceled)

7. (canceled)

8. (canceled)

9. (canceled)

10. (canceled)

11. (canceled)

12. The method of claim 1, wherein the composition comprises a therapeutic agent and a polymeric delivery vehicle.

13. The method of claim 12, wherein the polymeric delivery vehicle comprises a polymer selected from the group consisting of poly(lactic-co-glycolic acid), polyanhydrides, ethylene vinyl acetate, polyglycolic acid, chitosan, polyorthoesters, polyethers, polylactic acid, and poly (beta amino esters).

14. The method of claim 12, wherein the polymeric delivery vehicle comprises a poly(beta amino ester).

15. (canceled)

16. (canceled)

17. (canceled)

18. (canceled)

19. (canceled)

20. (canceled)

21. (canceled)

22. (canceled)

23. (canceled)

24. The method of claim 1, wherein the therapeutic agent comprises a polynucleotide.

25. (canceled)

26. (canceled)

27. (canceled)

28. The method of claim 24, wherein the composition comprises a polymeric delivery vehicle.

29. (canceled)

30. (canceled)

31. The method of claim 28, wherein the polymeric delivery vehicle comprises a poly (beta amino acid ester).

32. (canceled)

33. (canceled)

34. (canceled)

35. (canceled)

36. (canceled)

37. (canceled)

38. (canceled)

39. (canceled)

40. (canceled)

41. (canceled)

42. The method of claim 24, wherein the polynucleotide comprises a tissue-specific regulatory element operably linked to a nucleic acid that encodes a cytotoxic or cytostatic polypeptide.

43. (canceled)

44. The method of claim 24, wherein the polynucleotide comprises a tissue-specific regulatory element.

45. (canceled)

46. (canceled)

47. (canceled)

48. The method of claim 24, wherein the polynucleotide comprises:
(i) a tissue-specific regulatory element specific for the tissue, operably linked to a nucleic acid that encodes a site-specific recombinase;
(ii) a second regulatory element and a nucleic acid that encodes a cytotoxic or cytostatic polypeptide, wherein the second regulatory element is not operably linked to the nucleic acid; and
(iii) sites that are recognized by the site-specific recombinase and are so positioned that activity of the recombinase results in a recombination event that places the second regulatory element and the nucleic acid into operable linkage so that the nucleic acid is transcribed.

49. (canceled)

50. (canceled)

51. (canceled)

52. (canceled)

53. (canceled)

54. The method of claim 1, wherein the disease or condition is benign prostatic hyperplasia.

55. (canceled)

56. (canceled)

57. The method of claim 1, wherein the composition comprises a plurality of nanoparticles.

58. (canceled)

59. (canceled)

60. The method of claim 1, wherein the subject has not been diagnosed with cancer of the tissue that exhibits inappropriate or excessive noncancerous tissue growth.

61. A tissue-selective composition for the treatment of a disease or condition characterized by inappropriate or excessive noncancerous tissue growth, wherein the tissue-selective pharmaceutical composition comprises a therapeutic agent effective for treatment of inappropriate or excessive noncancerous tissue growth and does not comprise a viral delivery vehicle, and wherein the composition (i) comprises a tissue-selective delivery vehicle; (ii) comprises a polynucleotide; or (iii) comprises both a tissue-selective delivery vehicle and a polynucleotide.

62. The composition of claim 61, wherein the composition comprises a polymeric delivery vehicle.

63. The composition of claim 61, wherein the polymeric delivery vehicle is tissue-selective.

64. (canceled)

65. (canceled)

66. The composition of claim 61, wherein the polymeric delivery vehicle comprises a poly(beta amino ester).

67. (canceled)

68. (canceled)

69. (canceled)

70. (canceled)

71. (canceled)

72. (canceled)

73. (canceled)

74. (canceled)

75. (canceled)

76. The composition of claim 61, wherein the composition comprises a polynucleotide.

77. The composition of claim 61, wherein the composition comprises a tissue-specific therapeutic agent.

78. The composition of claim 77, wherein the tissue-specific therapeutic agent comprises a polynucleotide.

79. (canceled)

80. (canceled)

81. (canceled)

82. (canceled)

83. (canceled)

84. (canceled)

85. The composition of claim 78, wherein the polynucleotide comprises:
(i) a tissue-specific regulatory element operably linked to a nucleic acid that encodes a site-specific recombinase;
(ii) a second regulatory element and a nucleic acid that encodes a cytotoxic or cytostatic polypeptide, wherein the second regulatory element is not operably linked to the nucleic acid; and
(iii) sites that are recognized by the site-specific recombinase and are so positioned that activity of the recombinase results in a recombination event that places the second regulatory element and the nucleic acid into operable linkage so that the nucleic acid is transcribed.

86. (canceled)

87. A method for treating benign prostatic hyperplasia (BPH) comprising steps of:
providing an individual in need of treatment for BPH; and
administering to the individual a composition comprising a polynucleotide comprising a prostate specific regulatory element and a nucleic acid that encodes a cytotoxic or cytostatic polypeptide, wherein the composition either (i) does not comprise a viral delivery vehicle; or (ii) is locally delivered to noncancerous prostate gland tissue; or (iii) does not comprise a viral delivery vehicle and is locally delivered to noncancerous prostate gland tissue.

88. The method of claim 87, wherein the composition comprises a polymeric delivery vehicle.

89. The method of claim 88, wherein the polymeric delivery vehicle comprises a poly (beta amino ester).

90. (canceled)

91. (canceled)

92. (canceled)

93. (canceled)

94. (canceled)

95. The method of claim 87, wherein the prostate specific regulatory element comprises a regulatory element derived from a gene that encodes a protein selected from the group consisting of: PSA, PSMA, kallikrein 2, PSCA, probasin, and TARP.

96. The method of claim 87, wherein the prostate specific regulatory element comprises a promoter for PSA.

97. The method of claim 87, wherein the polynucleotide further comprises an enhancer.

98. (canceled)

99. (canceled)

100. The method of claim 87, wherein the polynucleotide comprises:
(i) a prostate-specific regulatory element operably linked to a nucleic acid that encodes a site-specific recombinase;
(ii) a second regulatory element and a nucleic acid that encodes a cytotoxic or cytostatic polypeptide, wherein the second regulatory element is not operably linked to the nucleic acid; and
(iii) sites that are recognized by the site-specific recombinase and are so positioned that activity of the recombinase results in a recombination event that places the second regulatory element and the nucleic acid into operable linkage so that the nucleic acid is transcribed.

101. (canceled)

102. (canceled)

103. The method of claim 87, wherein the cytotoxic or cytostatic peptide is a protein synthesis inhibitor.

104. The method of claim 87, wherein the cytotoxic or cytostatic polypeptide is selected from the group consisting of: diphtheria toxin A, gibbon ape leukemia virus fusogenic membrane glycoprotein, Pseudomonas exotoxin A (PE), cholera toxin (CT), pertussis toxin (PT), ricin A chain, abrin A chain, modeccin A chain, botulinum toxin A, alpha-sarcin, dianthin proteins, momordica charantia inhibitor, curcin, crotin, sapaonaria officinalis inhibitor, gelonin, mitogellin, hirsutellin A, calcaelin, restrictocin, phenomycin, and enomycin.

105. The method of claim 87, wherein the cytotoxic or cytostatic polypeptide is diptheria toxin A.

106. The method of claim 87, wherein the step of locally administering comprises injecting the composition into the prostate gland.

107. (canceled)

108. The method of claim 87, wherein the subject has not been diagnosed with prostate cancer.

109. A tissue-selective composition for the treatment of benign prostatic hyperplasia, wherein the tissue-selective composition comprises a therapeutic agent effective for treatment of BPH and does not comprise a viral delivery vehicle, and wherein the composition (i) comprises a tissue-selective delivery vehicle; (ii) comprises a polynucleotide; or (iii) comprises both a tissue-selective delivery vehicle and a polynucleotide.

110. The composition of claim 109, wherein the composition comprises a polymeric delivery vehicle.

111. The composition of claim 109, wherein the polymeric delivery vehicle is tissue-selective.

112. The composition of claim 109, wherein the polymeric delivery vehicle comprises a poly(beta amino ester).

113. (canceled)

114. (canceled)

115. (canceled)

116. (canceled)

117. (canceled)

118. (canceled)

119. (canceled)

120. The composition of claim 109, wherein the composition comprises a polynucleotide.

121. (canceled)

122. (canceled)

123. (canceled)

124. (canceled)

125. (canceled)
